SEQUENCE LISTING (247 sequences)

SEQ ID NO: 1 - DNA, 1719 bp, Klebsiella oxytoca
  source 1..1719: mol_type = genomic DNA
atgaaacagaccgtggcggcatacattgccaaaactcttgaacaggctggcgttaaacgtatttggggcgtaaccggggattcgctcaacggcttaagcgacagcctgaaccgtatggggaccatcgactggatgcccacgcgccatgaagaagtggccgccttcgccgccggggcggaagcgcagcttaccggcgaactggcggtctgcgcaggctcctgcgggccgggcaacctgcatctgattaacggccttttcgactgccaccgcaatcacgttccggttctggccatcgccgcccatattccttccagcgaaatcggcagcggctattttcaggaaacgcatccacaggagctattccgggaatgcagtcactactgtgaactggtctcaaccccggaacagatcccgcaggtgctggccatcgccatgcgcaaagcggtgattaaccgcggcgtctcggtagtggtgcttcccggcgacgttgcgctgaaagccgcgccggaaagcgccagcagccactggtaccacgcaccgctgccgcaggtgacgccggcggaagaagagctgaaaaagctggcccagctgctgcgctattcaagcaacatcgccctgatgtgcggcagcggctgcgccggggcgcataaagagctggtagacttcgccgcgaagctcaaagcgcctatcgttcacgcgctgcgcggcaaagagcacgtagaatacgacaacccttatgatgtggggatgaccggactgattggcttctcctccggcttccacaccatgatgaacgccgacaccctggtgctgctcggcacccagttcccctatcgcgctttctacccgacggacgcgaaaatcattcagattgatatcaatcccggcagtattggcgcccacagtaaagtggatatggcgctggtcggcgatattaaatcgaccctcagcgcgctgatgccgcatcttgaggagaaaaccgaccgtaagttcctcgataaagcgctggaacactatcgcgacgcgcgcaaggggcttgatgacctggcgaagcccagcgaaaaaacaattcatccgcagtatctggcgcagcgcatcagccactatgccgacgacgatgccatctttacctgtgacgtgggtacgccgaccgtctgggccgcgcgctatttgcagatgaacggtaagcgccgcctgctcggatcgttcaatcatgggtcaatggctaacgccatgccgcaggccatcggtgcgaaggccaccgcccctgaccgtcaggttgtggcaatgtgcggcgacggcggcttcagcatgctgatgggggattttctctcgctggcgcagatgaagctgccggtgaagatcgttatcttcaataacagcgtcctgggcttcgtggcgatggagatgaaggccgggggctatctgacagacggcactgaacttcacgccaccaactttgcccgcatcgccgaagcgtgcggcattaagggcatacgtgttgaaagcgcgtcagaggttgatgaagcgctgcaaaccgctttcgctaccgatggtccggtcctggtggatgtggtcgtcgccagtgaagagctggcgatgccgccgcaaattaaactggagcaggccaaaggtttcagcctttatatgctgcgcgcgattatcagcggccgcggcgacgaagttatcgaactggcgaaaactaactggctaaggtaa

SEQ ID NO: 2 - DNA, 2199 bp, Klebsiella oxytoca
  source 1..2199: mol_type = genomic DNA
gtggtgcagttttgtatcccgcctgatactggcggtaacgaaagaggatatatcgtgtcccgtacaatcatgctgatccctaccggaaccagcgtaggcctgaccagcgtcagcctcggtgttatccgtgctatggaacgtaaaggcgttcgtctgagcgtctttaaacctatcgctcaaccgcgtgccggtggcgatgcgccagaccagactaccactattattcgcgcgaactccgaccttccggccgcagaaccgctgaagatgagccacgttgagtcgctgctctccagcaaccagaaagacgtgctgatggaagagatcatcgccaactaccacgccaacgctcaggatgcggaagtggtgctggttgaaggtctggtcccgacccgcaaacatcagtttgcccagtctctgaactacgaaatcgcgaaaacgctgaacgctgaaatcgtttttgtgatgtcccagggtaccgatactcctgaacagctgaaagagcgcattgagctgactcgcagcagcttcggcggcgcgaaaaacaccagcgtcaccggcgtgatcgtcaacaagctgaatgcgccggttgacgaacagggccgtactcgccctgacctgtcggaaatcttcgacgactcttccaaagcaaaagtcgtgaagatcgatccggctcagctgcagagcggtagcccattaccggttctgggcgcggtgccgtggagcttcgatctgatcgccacccgcgcgattgatatggcgcatcacctgaacgccaccatcatcaacgaaggcgacatcaatacccgtcgcgtgaagtccgtcaccttctgtgcgcgtagcattccgcacatgctggaacacttccgcgcaggctccctgctggtcacttccgcagaccgtcctgacgttctggtcgccgcctgcctggccgcgatgaacggcgtggaaatcggcgctatcctgctgaccggcggctacgaaatggacgcccgcatcagcaaactgtgcgaacgcgcgtttgcaaccggcctgccggtattcatggttaacaccaacacctggcagacctctctgagcctgcagagcttcaacctggaagttccggttgacgatcacgagcgcatcgagaaagttcaggaatacgtggctaactatatcaacgccgactggattgaatcgctgaccgcaacttccgagcgcagccgtcgcctctctccgccagccttccgctatcagcttaccgagctggcgcgtaaagcaggcaagcgcgtcgtgctgccggaaggcaacgagccgcgtaccgttaaagccgcggctatctgcgccgagcgcggtatcgccacctgcgtactgctgggtaacccggatgaaatcacccgcgtggccgcctctcagggcgtagagctgggcgccggtattgaaatcgttgacccggaagtggttcgcgaaagctacgttgctcgcctggtcgagctgcgtaagagcaaggggatgaccgaagcggttgcccgcgagcagctggaagacaacgtggttctcggcaccctgatgctcgagcaggacgaagtcgacggcctggtatccggcgcggttcacaccaccgccaacaccatccgtccgccgctgcagctgatcaaaactgcgccgggcagctctctggtctcctccgtattcttcatgctgctgccggaacaggtttacgtttacggcgactgcgcgatcaacccggatccgaccgctgaacagctggctgaaatcgccatccagtccgcagactccgcaattgccttcggcatcgaaccgcgcgtggccatgctctcctactccaccggcacctccggcgccggcagcgatgtagagaaagtacgcgaagcgacccgtctggcgcaggaaaaacgtcctgacctgatgatcgacggcccgctgcagtacgatgcggcggtcatggctgacgttgcgaaatccaaagcgccgaactccccggttgccggccgcgctaccgtgtttatcttcccggatctgaacaccggtaacaccacttacaaagccgtacagcgttctgccgacctgatctccatcgggccgatgctgcaggggatgcgtaagccggtgaacgacctgtcccgtggcgcgctggttgacgatatcgtctataccatcgccctgaccgcgattcagtcgtcccagcagcaagcataa

SEQ ID NO: 3 - DNA, 1668 bp, Klebsiella oxytoca
  source 1..1668: mol_type = genomic DNA
atgcgcagccataccgttgctgctgaagggggttccgctgccgtcgcgcaggatcatgatagcttcgagtaccattttcacgacaccgttgccggcggcgactggctatgtgagcaggacgtcgttgactacttcgttcatcactgcccgacggaaatgacccagctcgaacagtggggatgcccgtggagccgccgcccggacggcagcgtcaacgtccggcgattcggcggcatgaagatcgaacgaacctggttcgctgccgataaaaccggctttcatatgctgcacaccctgttccagacctcgcttcaattcccgcaaatccaacgcttcgacgaacattttgttctggatattctggtcgatgatgggcaggctcgcggtctggtcgcgatgaatatgatggaaggtacgctggtgcagatccgcgccaacgcggtagttctggctaccggcggcgctggacgcgtgtatcgctacaataccaacggcggtatcgtcaccggcgacgggatgggtatggcgctcagccacggcgtaccgctgcgcgatatggagttcgtgcagtaccaccccaccggcctgccgggctccggtattctgatgacggaaggctgccgcggcgaaggcggtattctggtcaacaagaacggctaccgctacctgcaggattacggcatgggtccggaaacaccgctgggcgaaccgaaaaataaatatatggagctgggtccgcgcgataaagtctcccaggctttctggcacgaatggcgcaagggcaacacgatccctacgccgcgaggcgatgtggtctatctcgatctgcgccatttgggtgagaaaaagctgcttgaacgtctgccgtttatctgtgaactctcgaaagcctacgttggcgtcgatccggtaaaagagccgattccggtgcgtccaaccgcacactacaccatgggcgggattgaaaccgaccagcagtgtgaaacccgtatcaaagggctgtttgccgttggcgaatgttcttctgtgggtctgcacggcgccaaccgccttggctccaactccctggcggagctggtggtctttggtcgtctggccggtgagcaggccatgcagcgcgcaaccgaagcgggcgaagccaatagcgccgcgctggacgcgcaggtcgtcgatatcgagaagcgcctgaaagacctggttaatcaggaaggtaacgagaactgggcgaagattcgcgacgacatgggtctgtcgatggaagaaggctgcggcatctaccgtacgccggagctcatgcagaaaacggtcgataagctcgccgagctgcaggagcgcttcaagcgcgtgcggatcaccgacacgtccagcgtgttcaataccgacctgctgtacaccatcgagctgggccatggcctgaacgtcgccgaatgtatggcgcattccgccctcgcgcgtaaagagtcccgcggcgcgcatcagcgcctggatgaaggctgcaccgagcgcgacgacgtcaacttcctcaagcataccctcgccttccgcgatgccgatggtcacaccaggctggagtacagtgatgtgaaaatcaccactctgccgccggcaaaacgcgtgtacggtgcggaagcggaagcagccgagaagaaggagacgacgaatggctga

SEQ ID NO: 4 - DNA, 990 bp, Klebsiella oxytoca
  source 1..990: mol_type = genomic DNA
atgaaaatcgctgtatacagtacgaaacagtacgacaagaagtatctgcagcacgtcaatgatgcatacggctttgaactggaattttttgacttcctgctaaccgaaaagaccgccaaaaccgccaacggctgtgaagcggtgtgcatcttcgtaaacgatgacggtagccgcccggtactcgaagaactgaaagcccacggcgtgaagtacatcgcgctgcgctgcgcgggcttcaacaacgttgatctcgatgccgcgaaagagctgggcctgcgcgtggtgcgcgtcccggcctactcgccggaagcggtcgcagagcacgcgatcggcatgatgatgtcgctgaaccgccgtattcatcgcgcctatcagcgcacccgcgacgcgaacttctcccttgaaggactaaccggtttcaccatgcacggtaaaaccgccggcgttatcggcaccggtaaaatcggcgtcgccgcgctgcgcattctgaaaggcttcggtatgcgtctgctggcgtttgatccctacccaagcgccgccgcgctggatatgggcgtggagtatgtcgatcttgaaaccctgtaccgggagtctgacgttatctccctgcactgcccgctgaccgatgaaaactaccatttgttgaaccatgccgcgttcgatcgcatgaaagacggggtgatgatcatcaacaccagccgcggcgcgctcatcgattcgcaggcagcaatcgacgcccttaagcatcagaaaattggcgcgctggggatggacgtgtatgagaacgaacgcgatctgttctttgaagataagtctaatgacgtgattcaggatgatgtgttccgccgtctctctgcctgccataacgtcctgtttaccggtcaccaggcgtttctgaccgcggaagcgctgatcagcatttcacaaaccaccctcgacaacctgcgtcaggtggatgctggcgaaacctgccctaacgcgctggtctga

SEQ ID NO: 5 - DNA, 2283 bp, Klebsiella oxytoca
  source 1..2283: mol_type = genomic DNA
atgtccgagcttaatgaaaagttagccacagcctgggaaggttttgcgaaaggtgactggcagaacgaagtcaacgtccgcgacttcatccagaaaaactataccccgtacgaaggtgacgagtccttcctggctggcgcaactgacgcgaccaccaagctgtgggacaccgtaatggaaggcgttaaacaggaaaaccgcactcacgcgcctgttgattttgatacttcccttgcatccaccatcacttctcatgacgctggctacatcgagaaaggtctcgagaaaatcgttggtctgcagactgaagctccgctgaaacgcgcgattatcccgttcggcggcatcaaaatggtagaaggttcctgcaaagcgtacaatcgcgaactggatccgatgctgaagaaaatcttcactgaataccgcaaaactcacaaccagggcgtgtttgacgtttacaccaaagacatcctgaactgccgtaaatctggcgttctgaccggtctgccggatgcctatggccgtggtcgtatcatcggtgactaccgtcgcgttgcgctgtacggtatcgacttcctgatgaaagacaaatacgctcagttcgtctctctgcaagagaaactggaaaacggcgaagatctggaaggaactattcgtctgcgcgaagaaatctctgaacagcaccgtgcgctgggtcagatcaaagaaatggcggcaaaatacggctgcgatatctctggtccggcaaccaccgctcaggaagctatccagtggacctacttcggctacctggctgccgttaaatctcagaacggcgcggcaatgtccttcggtcgtacctccagcttcctggacatcttcatcgaacgtgacctgaaagccggtaaaatcaccgagcaagacgcacaggaaatgattgaccacctggtcatgaaactgcgtatggttcgtttcctgcgtacccctgaatatgatgaactgttctctggcgacccgatttgggcaaccgaatctatcggcggtatgggcgttgacggccgtactctggtcaccaaaaacagcttccgtttcctgaacaccctgtacaccatggggccgtctccggagccgaacatcaccattctgtggtctgaaaaactgccgctgagcttcaaaaaatacgccgcgaaagtgtccatcgatacctcttctctgcagtacgagaacgatgacctgatgcgtcctgacttcaacaacgatgactacgctatcgcttgctgcgtaagcccgatggttgttggtaagcaaatgcagttcttcggcgcgcgtgctaacctggcgaaaaccatgctgtacgcaatcaacggcggcgttgatgaaaaactgaaaatgcaggttggtcctaaatctgaaccgatcaaaggcgacgttctgaacttcgacgaagtgatggaccgcatggatcacttcatggactggctggctaaacagtacgtcactgcgctgaacatcatccactacatgcacgacaagtacagctacgaagcttccctgatggcgctgcacgaccgtgatgttatccgcaccatggcatgtggtatcgcaggtctgtccgttgcggctgactccctgtctgcaatcaaatatgcgaaagttaaaccgattcgtgacgaaaacggtctggctgtcgacttcgaaatcgaaggcgaatacccgcagtttggtaacaacgactctcgcgtcgatgatatggccgttgacctggttgaacgtttcatgaagaaaattcagaaactgcacacctaccgcaacgctatcccgactcagtccgttctgaccatcacctctaacgttgtgtatggtaagaaaaccggtaacacccctgacggtcgtcgcgctggcgctccgttcggaccaggtgctaacccgatgcacggccgtgaccagaaaggtgctgttgcctctctgacctccgttgcgaaactgccgtttgcttacgcgaaagatggtatttcttacaccttctctatcgtgccgaacgcgctgggtaaagacgacgaagttcgtaaaactaacctcgccggcctgatggatggttacttccaccacgaagcgtccatcgaaggcggtcagcatctgaacgtcaacgttatgaaccgcgaaatgctgctcgacgcgatggaaaatccggaaaaatatccgcagctgaccatccgcgtatccggctacgcagtacgttttaactccctgacgaaagagcagcagcaggacgttattactcgtaccttcactcagaccatgtaa

SEQ ID NO: 6 - DNA, 1728 bp, Paralcaligenes ureilyticus
  source 1..1728: mol_type = genomic DNA
atgagtgacaaagaaaagcctgtgcgtcgcagccaggcctggttcggacggcttgaccgcgacggtttcatctaccgttcgtggatgaagaatcgaggcatcccgcacgatcagttcgacggtcgcccggtgattggcatttgcaatactttctctgaactgacgccgtgcaattcgcatttccgcaccttggccgagcaggtcaagatcggggtttgggagtcgggcggcttccctctcgagtttccggtcatgtcgctgggcgagacgatgttgcggcctacggccatgctgtttcgcaacctggccagcatggacgtcgaggaatcgatacgcggcaatccgttggatggcgtcgtattgctgatgggctgcgacaaaacgacgccttcgctcatgatgggggcggcgtcctgcgacctgcccactatcggtgtgtcgggcggtcccatgcttagcggcaagtttcgaggccgcgaactgggttcaggcaccgacgtctggaagatgtctgaagaagttcgcgccgggcaaatgtcgcaggaagaatttttcgaagcggaaagctgcatgcatcgctcgcatgggcattgcatgacgatgggtacggcatccacgatggccagcatggtggaagcgctgggcatgagcctgcctggcaacgcggcgattcccgccgtggatgcgcgccgcaatctgctcgcgcgcgcttcgggccggcgcattgtgcagatggtcaaagatgatctggtcatgtcgaaaatcctgacgcgccaggcctttgaaaacgccatccgcgtgaacgccgctatcggtggctcgaccaacgcggtgattcatctgttggcgattgcggggcgtatcggggttgacctgacgcttgccgactgggatgcgctcggccacaaattgccgtgcctggttgatctgcagccttcgggcactcatctgatggaagacttttattatgcgggcggcgtgcccgccgtgatccgcgagctgggcgatgtcattgcccgcgatgccctgacggtgaatggccagaccctgtgggataactgcaaggatgcgccaaactggaatcgcgaggtcatccatgcctttaacgagcctttcaagaccgaggcggggatagccgtactgcgtggcaacctatgccccgatggggccgtcatcaaaccttcggccgccacgccggccctgctcaagcacaagggccgcgcggtcgtgttcgaaaacagcgaacatatgcacgagcgcatggacgatgaaaacctggacgtcgacgagaattgcgtgctggttttgaagaattgcgggccccgcggctatccgggtatggccgaagccggcaatatgccgctgccgccgaaaatcctgcgcaaaggcatcaccgatatggtccgggtgtccgacgcccgcatgagcggcaccgcgtatggcacggtggttctgcatgtggcccccgaggccgccgcgggcggccctttggcgctggtgcaggacggcgacatcatcgaactggatgtggccgcgcgcaagctgcatttgcatgtgtccgatgaagagctcgcacgccgccgcgaggcatggcaagcgccgccggcgcccatggcgcgcggctgggtcaagctttatgtcgagcatgtgcagcaagccaatctgggcgccgaccttgattttctgcgaggtaaaagcggcgcgggcatacccaaagacaatcactaa

SEQ ID NO: 7 - DNA, 1095 bp, Bacillus subtilis
  source 1..1095: mol_type = genomic DNA
atggaactttttaaatatatggagaaatacgattacgaacaattggtattctgccaggatgaacaatctggattaaaagcgattatcgccattcatgatacaacgcttggtccggcgcttggcggaacgagaatgtggacatatgaaaatgaagaagcggcaattgaagatgcgctcagattggcaagaggcatgacctataagaacgcggcggcaggcttaaaccttggcggcggaaaaacagtcattatcggcgatccgcgcaaagacaaaaatgaggaaatgttccgcgcgtttggccgctatattcaaggactgaatggcagatacatcacggctgaagatgtgggcacaacggtcgaggatatggatatcattcatgatgagacagactatgtcacagggatttctcctgctttcggctcttctggaaatccgtccccagtcacagcgtacggggtgtacagaggaatgaaggcagcagctaaagctgctttcggaaccgactctcttgaaggaaaaaccattgctgtacagggtgttgggaacgtagcctataacctttgccgccacctgcatgaagaaggagcaaacttaatcgttacggatatcaacaaacaatctgtacagcgtgcagttgaagattttggcgcccgtgcggtagatcctgatgacatttattcacaagactgcgatatttatgcgccgtgtgcccttggtgcgactattaacgacgacaccattaaacagctgaaggcgaaagtgatcgcaggtgcggctaacaaccaattaaaagagacacgccatggtgatcaaattcacgaaatgggcatcgtttatgcaccggattacgtgattaacgcgggcggtgtcatcaacgtggcagatgagctttacggctataatgcagaacgtgcattgaaaaaagttgaaggcatttacggcaatatcgagcgtgtacttgagatttctcagcgtgacggcattcctgcatatttagcggctgaccgcttagcagaggaacggattgaacgcatgcgccgctcaagaagccagtttttgcaaaacggccacagtgtattaagcagacgttaa

SEQ ID NO: 8 - DNA, 2676 bp, Klebsiella oxytoca
  source 1..2676: mol_type = genomic DNA
atggctgttactaatgtcgctgaacttaacgcactcgtcgagcgcgtaaaaaaagcccagcgtgaatatgccagtttcactcaagaacaagtagacaaaatcttccgcgccgccgctctggccgctgcagatgctcgaatccctctcgccaagatggccgttgccgaatccggcatgggcatcgtcgaagataaagtgatcaaaaaccactttgcttccgaatacatttataacgcctataaagatgaaaagacctgcggcgtcctgtctgaagatgacaccttcggtaccatcaccatcgctgagccgattggtattatctgcggtatcgtcccgaccactaacccaacttcaaccgctatctttaaatctctgattagcctgaagacccgtaacgccatcatcttctctccgcatccgcgtgctaaagatgcgaccaacaaagcggctgatatcgtactgcaggcagctatcgcagccggtgcgccgaaagatctgattggttggattgaccagccttccgttgaactgtcaaatgcgctgatgcaccatccggatatcaacctgatccttgcgaccggcggtccgggcatggttaaggcggcgtacagctccggtaaacctgccatcggcgtaggcgcaggtaacacgcctgttgttatcgatgaaacggctgatatcaagcgtgccgttgcttccgtcctgatgtctaaaaccttcgataacggcgttatctgtgcttctgagcaatccgttgtggttgtagattccgtatacgatgccgttcgcgaacgtttctccagccacggcggctatctgctgcagggccaggagctgaaggctgttcagaatatcatcctgaaaaacggcgccctgaacgccgcgatcgtaggtcaaccggcgtataaaatcgctgaactggccggcttcaccgtccctgtttccaccaaaattctgattggtgaagtcaccgacgttgatgaaagcgaaccgtttgcccacgaaaaactgtctccgacgctggcaatgtaccgtgcgaagaacttcgaagacgcggttgataaagctgaaaaactggtggcaatgggcggtatcggtcatacctcttgcctgtataccgaccaggacaaccagccagaacgcgttgcttacttcggtcagctgatgaaaaccgcacgtatcctgatcaacacccctgcttctcagggtggtatcggtgacctgtacaacttcaaactcgcaccttccctgactctgggttgtggttcatggggtggtaactccatctctgaaaacgttggtcctaagcacctgatcaacaagaaaaccgttgctaagcgagctgaaaacatgttgtggcacaaacttccgaaatctatctacttccgtcgtggctcactgccaatcgcgctggatgaagtgattactgatggtcacaaacgcgcgctgatcgtgaccgaccgcttcctgttcaacaacggctatgctgaccagatcacctctgtgctgaaagcggctggcgttgaaaccgaagttttctttgaagtggaagctgacccgacgctgaccatcgttcgtaaaggcgctgagctggcgaactccttcaaaccagacgtgattatcgcgctgggcggcggttccccgatggatgcggcgaaaatcatgtgggttatgtacgagcacccggaaacccacttcgaagaactggcgctgcgctttatggatatccgtaaacgtatctacaagttcccgaaaatgggcgtgaaagccaaaatggtcgccatcaccacgacttccggtaccggttctgaagttacgccgttcgccgttgttaccgatgatgcaaccggtcagaaatacccgctggcagactatgccctgactccggatatggcgattgttgatgccaacctggtcatggaaatgccgaagtcactgtgtgctttcggtggtctggatgcggtgactcatgccctggaagcttacgtttccgtactggcttccgagttctctgacggccaggcgctgcaggctctgaaactgctgaaagaaaacctgccagcgtcctaccacgaaggttctaagaacccggtagcgcgtgagcgtgtgcacagtgctgcgaccatcgccggtatcgcgtttgcgaacgccttcctcggggtatgtcactcaatggcgcacaagctgggctctcagttccacattcctcacggtctggcgaacgccctgctgatcagcaacgttattcgctataacgctaacgacaacccgactaaacagacagcattcagccagtacgaccgcccgcaggcacgtcgtcgctacgctgaaatcgcggaccatctgggtctgactgcaccgggtgaccgtaccgcagcgaaaatcgaaaaactgcttggctggttggacgaaatcaaagctgagctgggtattcctaagtctatccgcgaagctggcgtgcaggaagctgatttcctggcccacgttgacaagctgtctgaagacgcatttgacgaccagtgcaccggcgctaacccgcgctacccgctgatcgctgagctgaaacagattctgctggatacgttctacggacgtaattacgttgaaggcagcgtggaagagaaaaaagaagtcgcgccggctaaagctgagaagaaagcgaaaaaatccgcttaa

SEQ ID NO: 9 - DNA, 1476 bp, Escherichia coli
  source 1..1476: mol_type = genomic DNA
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctgagtttaaagaagcgattgccgaggagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 10 - DNA, 780 bp, Klebsiella oxytoca
  source 1..780: mol_type = genomic DNA
atgaaccattctgctgaatgctcttgcgaagagagcctgtgtgaaactctacgaggattttccgcgcaacatcccgatagcgtcatctaccagacttctctgatgagcgcgctgttgagcggcgtttatgagggcaataccaccatcgccgatttgctcacccacggcgatttcggcctgggcacctttaatgaactggacggcgagctgatcgcgtttagcagcgaggtttaccagctgcgcgccgacggcagcgcccgtaaagcccgaatggaacagcgcacgcccttcgcggtgatgacctggtttcagccgcagtaccgcaaaacgtttgataaaccggtcagccgcgaacagctgcacgacattatcgaccggcaaatcccctccgataatctgttctgcgccctgcgtatcaacggccattttcaccacgcccatacccgcaccgtaccgcgccagacgccgccctaccgggcgatgaccgacgtgctcgacgaccagcccgttttccgcttcaaccagcgcgaaggggtgctggtggggtttcgcacgccgcagcatatgcagggcattaacgttgccggctaccacgaacacttcatcaccgatgaccgccagggcggcggccatctgctcgattatcagctcgaccacggcgtgctgacctttggtgagatccacaaattgatgattgaccttcctgccgatagcgccttcctgcaggcggatctgcatcctgacaatcttgatgccgccattcgctcagtcgaaaactaa

SEQ ID NO: 11 - DNA, 1079 bp, Corynebacterium glutamicum
  source 1..1079: mol_type = genomic DNA
gtgcaaaaaacgcaagagattcattcaagcctggaggtgtcgccatccaaggcagccctggaaccagatgataaaggttatcggcgctacgaaatcgcgcaaggtctaaaaacctcccttgctgcaggtttgggcatgtacccgattggtattgcgtttggtctcttggttattcaatacggctacgaatggtgggcagccccactgttttccggcctgattttcgcgggctccaccgaaatgctggtcatcgccctcgttgtgggcgcagcgcccctgggcgccatcgcgctcaccacattgctggtgaacttccgccacgtattctatgcgttttcattcccgctgcatgtggtcaaaaaccccattgcccgtttctattcggttttcgcgcttatcgacgaagcctacgcagtcactgcggccaggcccgcaggctggtcggcgtggcgacttatctcaatgcaaatagcgtttcactcctactgggtattcggcggtctcaccggagtggcgatcgcagagttgattccttttgaaattaagggcctcgagttcgccctttgctctctctttgtcacgctgactttggattcctgccgaacgaaaaagcagatcccttctctgctgctcgcaggtttgagcttcaccattgctcttgtggtaattccaggtcaggccctatttgcggcgctgctgatcttcttgggtctgttgaccatccggtacttcttcttgggaaaggctgctaaatgacaactgatttctcctgtattctccttgttgtcgcagtatgtgcagtcattacttttgcgctccgggcggttccgttcttaatccttaagcccctacgtgaatcacaatttgtgggcaaaatggcgatgtggatgccagcaggaatccttgccattttgaccgcatcaacgtttcgcagcaatgcgatagatctgaagactctaacctttggtctcattgccgttgcgattacagtggtggcgcatcttcttggcggtcgacgcaccttgttgagcgttggcgctggcaccatcgtttttgttggactggtgaatcttttctaa

SEQ ID NO: 12 - DNA, 1713 bp, Bacillus subtilis
  source 1..1713: mol_type = genomic DNA
ttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 13 - DNA, 771 bp, Klebsiella oxytoca
  source 1..771: mol_type = genomic DNA
atgaaaaaagtcgcactcgtgaccggcgcgggccagggcatcgggaaagctatcgcccttcgcctggttaaagatggttttgccgtggttatcgccgattacaatgacgccaccgcacaggcggtcgccgatgaaattaaccgcggcggcggtcaggcgctggcggtgaaggtggatgtctctaaacgcgaccaggtttttgccgccgtagaacaggcgcggaagggcctgggcggttttgacgtgattgtgaacaacgccggggtggccccctccacgcctatcgaagagattcgcgaggacgtgatcgataaagtctacaatatcaacgtcaaaggcgttatctggggtatccaggccgcggtagaggcgtttaaaaaagagggccacggcggcaaaatcatcaacgcctgctcccaggcgggccacgtgggtaacccggaactggcggtctatagctcaagtaagtttgccgtgcgcggcctgacgcaaaccgccgcccgcgatctggcgcatctgggaattaccgttaacggctactgcccggggatcgttaaaaccccaatgtgggcggaaatcgaccgtcaggtttccgaagcggcgggtaaaccgctgggctacgggactcaggagttcgccaaacgcattacccttggacggctttcagagccggaagacgtcgcggcctgcgtctcttatctcgccggtccggactccagctatatgaccggccaatcgctgctgatcgatggcggcatggtatttagctaa

SEQ ID NO: 14 - DNA, 1476 bp, Escherichia coli
  source 1..1476: mol_type = genomic DNA
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 15 - DNA, 1104 bp, Klebsiella oxytoca
  source 1..1104: mol_type = genomic DNA
atggatcgcattattcaatcacccggtaagtacattcagggcgcggacgtcattacccgccttggtggatacctaaagccgctggcagaacgctggctcatcgtgggcgataaattcgtactcggttttgccgaagaaaaactgcgtaaaagcctggcggatgcggggctggctggcgaaatcgcaccgtttggcggcgaatgctcacataacgaaatcaaccgcttgcgtgatattgccggttccgcccgctgcaccgccgtgctgggtatcggcggcggcaagacgctggataccgcaaaagcattagcacactttatgaatctgccggtggttattgccccgacgattgcctccaccgacgcgccgtgcagcgcgctctcggtcatctataccgatgacggcgagtttgatagctacctgatgctgccgcgcaacccgaacatggtcattgtcgatacgcaaatcgttgcaggcgccccggcgcgcctgctggcagccggtatcggtgatgcgctggcgacctggtttgaagcgcgtgcctgctcccgcagcggcgcgaccaccatggcgggcggaaaatgcacccaggcggcgctggcgctggctgaactgtgctacaacacgctggttgaagagggcgaaaaagcgatgctggcggcggagcagcacgtggtgacgccggcgctggaacgggtgattgaagccaacacctatctgagcggcgtcggctttgaaagcggcggcctggcggctgcgcacgctatccataacggtctgaccgctattccggacgctcaccactactatcatggtgaaaaagtggctttcggtacgcttacgcagctggtactggaaaacgcgccggtggaagagatcgaaaccgtagcggcgctgtgccatagcgtcgggctgcctatcaccctggcgcagctggatattaaagtcgatatcccgggcaaaatgcgtaccgtggcggaagccgcctgcgcggaaggtgaaaccatccacaatatgcccggcggcgcgacgccggatcaggtctatgccgcgcttctggtggccgaccagtacggtcaacgcttcctgcaagagtgggaataa

SEQ ID NO: 16 - DNA, 1677 bp, Sulfolobus solfataricus
  source 1..1677: mol_type = genomic DNA
atgccggcaaaactgaactctccaagccgttaccacggcatctacaacgctccgcaccgtgcatttctgcgcagcgtcggtctgaccgacgaagaaatcggtaagccgttagtggctatcgccaccgcctggtccgaagcgggcccgtgtaactttcataccctggcgctggctcgtgtggcaaaggaaggcacgaaagaggctgggctgagcccgctggctttcccgactatggtcgtgaacgataacatcggcatgggctccgagggcatgcgttactccctggtgtcgcgtgacctgattgcagatatggtggaagctcagttcaacgcgcatgcattcgacggtctggtcggcatcggtggctgcgataaaactacgcctggtatcctgatggctatggcgcgcctgaacgtgccatccatctacatctacggtggttccgcggaaccgggttacttcatgggtaagcgtcttactatcgaggacgtgcacgaagcgatcggtgcgtacctggcgaaacgcatcaccgagaacgaactgtacgaaattgaaaaacgtgctcacccaaccctggggacttgttccggcctgtttacggctaacaccatgggctccatgtctgaagccctgggcatggcgctgccaggcagcgcctctcctaccgcaacgagcagccgtcgtgttatgtacgtcaaggaaaccggcaaggcgctgggttctctgatcgaaaacggcattaagtcacgtgaaatcctgaccttcgaagcattcgaaaacgcgattaccaccctgatggctatgggcggctccaccaatgcagtgctgcatctgctggcgatcgcctacaaggctggtgttaaattcaccctggacgacttcaaccgtatctccaagaggactccgtacattgcgtccatgaaaccgggtggggactacgtcatggcagatctggatgaagtcggcggtgtcccggtcgtgctgaaaaagctgctggatgccggtcttctgcacggtgatgttctcactgtaaccggcaaaactatgaaacagaatctggaacagtataaatacccgaacgttccgcactcccacatcgtacgcgacgtgaaaaaccctattaaaccgcgtggtggcatcgtaattctgaaaggctctctggcgccggaaggcgctgtcattaaagttgctgctactaacgttgttaagtttgaaggtaaagccaaagtatataacagcgaggacgacgcttttaaaggcgttcaaagcggcgaagtcagcgaaggcgaggtagtaattatccgctacgaaggtccgaaaggggcgccgggcatgccggaaatgctgcgcgtgacagcggcaatcatgggtgccggcctgaacaacgttgccctggtgaccgacggtcgtttttctggtgcgactcgtggcccgatggttggtcacgttgctccggaagccatggtgggtggtccaatcgccatcgttgaagatggcgatacgatcgtgatcgacgtggaatcagaacgcctggatctgaaactgtctgaagaagaaatcaagaaccgcctgaaacgttggtccccgccgagcccgcgttacaagtccggtctgctggcaaaatacgcgagcctggtttctcaggcgagcatgggcgcggttacgcgtccggcataa

SEQ ID NO: 17 - DNA, 1851 bp, Escherichia coli
  source 1..1851: mol_type = genomic DNA
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 18 - DNA, 978 bp, Enterobacter cloacae
  source 1..978: mol_type = genomic DNA
atgagcgaaacggccacctggcagccgagcgcatccatccctaatctgctgaaacgcgctgcaattatggcggagatccgccgcttctttgctgaccgcggagtccttgaggtggaaacgccgtgcatgagtcaggcaacggtaacggatattcatctggtaccgtttgaaacccgttttgttggccccggccattcacagggcatgaacctctggctgatgaccagcccggaataccacatgaaacgcctgctggcggcagggtgtggcccggtgtatcagctgtgccgcagcttccgcaatgaagagatggggcgtcaccataatccggaattcaccatgctggagtggtaccgtccgcattacgatatgtaccgcctgatgaacgaggtggacgatctgctgcagcaggttctggactgctcagaagcggaaacgctctcttatcagcaggctttccagcgttatctggaaattgatccattgtcagcggataaaacccagctgcgcgaagtggcggcaaaactggatctcagcaacgtggcggataccgaagaagatcgcgacacgctgctgcaactgcttttcacctttggtgttgaaccgcagattggtaaagatcgtcccactttcgtctaccacttcccggcaagccaggcttctctggcgcagatcagcactgaagatcaccgcgtggcggagcgttttgaggtctattacaagggcattgagctggcgaatggtttccacgaactgaccgacgcccgcgaacagcagcagcgttttgagcaggataaccgcaagcgcgccgcgcgcggtctgccgcagcagcctattgatactaacctgctggaagccctgaaagcgggtctgccggattgttccggtgtggcgttgggggtggatcgtctggtgatgctggcgctgggcgcggaacagctgggcgatgtgattgcttttaccgtcgatcgagcctga

SEQ ID NO: 19 - DNA, 2142 bp, Enterobacter cloacae
  source 1..2142: mol_type = genomic DNA
gtgtcccgtactattatgctgatccctaccggaaccagcgtcggcctgaccagcgtcagccttggcgtgatccgtgctatggaacgcaaaggcgttcgtctgagcgtctttaagccaatcgcccagccacgtgccggtggcgatgcgccagaccagaccaccaccattgttcgtaagaactccaatctgccagcggctgaaccgctgaagatgaaccacgttgaatctctgctctccagcaaccagaaagacgtgctgatggaagagatcatcgccaactaccacgccaatacaaaagacgcggaagtggtgctggttgaaggcctggttccgacccgcaaacaccagtttgcccagtcgctgaacttcgaaattgcaaaaaccctgaacgccgagatcgtctttgtgatgtctcagggcacagataccccagagcagctgaaagagcgtatcgaactgacccgcagcagcttcggtggtaccaaaaacaccaacatcacgggcgtaatcgtcaacaaactgaatgcgcctgtggatgagcagggccgtacgcgtcctgacctgtccgagatcttcgacgactcttccaaagcgaaagtcattaaagttgacccggctaaactgcaggaatccagcccgctgccggttctgggcgcggtgccgtggagcttcgatctgattgccacccgtgccatcgatatggcgcgtcacctgaacgccaccgtgatcaacgaaggcgacatcaatacccgccgcgtgaaatccgtgaccttctgcgcccgcagcattccacacatgctggaacacttccgtgcaggttccctgctggtgacctccgcagaccgtccagacgtgctggttgctgcctgcctggccgcgatgaatggcgttgaaatcggcgctatcctgctgaccggtgcttacgagatggatccacgcgtcagcaagctgtgcgaacgcgcgttcgcgactggcctgccggtattcatggtcaacaccaacacctggcagacctccctgagcctgcagagcttcaacctggaagtgccggttgatgaccacgagcgtatcgagaaggttcaggaatacgttgcaagctacatcaacgctgactggatcgaatccctgaccgcaacctccgagcgcagccgtcgtctgtctcctccggccttccgttaccagctgaccgagctggcgcgtaaagcgggcaaacgtgttgttctgccagaaggcgatgaaccacgtaccgtgaaagcggctgccatctgtgcagagcgcggcatcgcaacctgtgtgctgctgggtaacccggatgagatcaaccgtgttgcggcgtctcagggcgttgagctgggcgctggcatcgaaatcgttgatccagacgtggttcgcgaaagttatgttgctcgtctggttgaactgcgtaagagcaagggcatgaccgaagccgttgcgcgtgagcagctggaagacaacgtggtactgggtacgctgatgctggaacaggacgaagttgacggtctggtgtccggtgcggttcacactaccgcgaataccatccgtccaccgctgcagctgatcaaaacggctccaggcagctctctggtttcctccgtgttcttcatgctgctgcctgaacaggtttacgtttacggcgactgtgcgatcaacccggatccaactgcagagcagctggctgagatcgcgattcagtctgcggactccgcgattgccttcggtatcgaaccgcgcgtcgcgatgctctcctactccaccggcacctctggtgcaggtagcgatgtagagaaagtgcgtgaagcgacccgtattgcacaggaaaaacgtcctgatctgatgatcgacggcccgctgcagtacgatgccgccgtaatggctgatgttgcgaaatccaaagcgcctaactctccggttgcgggtcgcgctaccgtgttcatcttcccggatctgaacaccggtaacaccacctacaaagcggtacagcgttctgccgacctgatctccatcgggccaatgctgcagggtatgcgcaaacctgtgaacgacctgtcccgtggcgcgctggtagacgatatcgtctacaccatcgcattgaccgcgatccagtcttcacagcagcaataa

SEQ ID NO: 20 - DNA, 1791 bp, Enterobacter cloacae
  source 1..1791: mol_type = genomic DNA
gtgcaaacttttcaagccgatcttgccgtaataggcgctggcggggctggattacgtgctgcgattgctgcagcacaggctaatcccaacgctaaaatcgcactgatttcaaaagtctatccgatgcgcagccacacggttgccgcagaaggaggatccgccgccgttgcgcaggatcatgacagcttcgaataccatttccacgacacggttgcagggggcgactggctttgcgaacaggatgtcgttgactactttgtacaccactgtccaacggagatgacccagcttgaacagtggggatgcccgtggagccgccgtccggacggcagcgttaacgttcgccgcttcggcggaatgaaaatcgaacgcacctggtttgccgccgataagaccggcttccacatgctgcataccctattccagacctcccttcagttcccacaaattcagcgctttgacgaacattttgtcctcgatattctggtcgatgacggtcatgcgcgcggtctggtggccatgaacatgatggaaggcacgctcgtccagatccgcgctaacgcggtggtgatggcaacgggcggtgcgggccgggtttaccgttacaacaccaacggcggcatcgtcaccggtgacggtatgggtatggcgctcagccacggcgtgccgctgcgcgatatggaattcgtccagtatcaccctaccggcctgccaggctccggcatcctgatgacggaaggctgccgcggcgaaggcggtattctggtcaacaagaacggctaccgttatctgcaggattacggcatgggcccggaaaccccgctcggtgagccgaaaaacaaatacatggagctcggcccgcgcgacaaagtgtctcaggccttctggcatgagtggcgcaaaggcaacacgatcgctacgccacgcggcgatgtggtctacctcgacctgcgtcacctcggcgagaagaaactgctggaacgtctgccgttcatctgcgagctggcaaaagcctacgtgggtgttgacccggtgaaagagccaattccggtacgccccacagcgcactacaccatgggcgggattgaaaccgaccagcagtgtgaaacgcgcatcaaagggttgttcgccgtcggcgagtgctcctctgtcggcctgcacggtgccaaccgtctcgggtcaaactcgctggctgagctggttgtctttggccgcatggcgggcgaacgcgcgatggagcgtgccgcaacaacgggcgaagccaacggcgcagcactggacgcacaggtcgctgacgtcgagaaacgactgaaagacctggtcaaccaggaaggtaacgaaaactggtcgaaaatacgcgacgaaatgggtatgtcgatggaagaaggctgcggtatctaccgtacgccagagctgatgcagaaaaccgtcgataagctggcggaactgcaggagcgtttcaaacgcgtgcgcatcaccgatacctccagcgtattcaacaccgacctgctttataccatcgagctcggtcacggcctgaacgtcgcggaatgcatggcgcactccgccctggcacgcaaagagtcacgcggggcgcatcagcgtcttgatgagggctgtaccgaacgtgacgacgtcaatttcctgaaacacaccctcgcctggcgcgacgcggatggcaccactcgtctggactacagcgacgtgaagatcacaacgctgccaccggctaaacgcgtttacggcgcagaagcagaagccgccgagaagaaggagaaggcgaatggctga

SEQ ID NO: 21 - DNA, 990 bp, Enterobacter cloacae
  source 1..990: mol_type = genomic DNA
atgaaactcgcggtatatagcacaaagcagtacgacaaaaagtatctgcaacatgttaacgaggcttacgggttcgatcttgaatttttcgactttctgctgaccgaaaaaaccgctaaaacggcgcacggctgtgaaggcgtctgcatcttcgtgaacgatgacggtagtcgtccggtactggaagagctgaaaaagcacggcgtaaagtatatcgccctgcgctgcgcgggcttcaataacgtcgatctggatgcggcgaaagagctggggctgaaggtggttcgcgttccggcctactccccggaagcggtcgcggaacatgctgtgggcatgatgatgtccctgaaccgtcgtattcaccgcgcttatcagcgtacccgagatgcgaacttctcgctggaaggactgaccggctttaccatgtacggtaaaaccgcaggcgtgatcggtaccggtaaaatcggcgtggccgccctgcgcattctgaaaggctttggtatgcgcctgctggcgtttgacccgtacccaagcgccgccgcgctggagctgggcgtggaatatgtcgatctcccgaccctgttctcccagtctgacgtgatctccctgcactgcccgctgacgccggaaaactatcacctgctgaatcagaccgcgtttgagcagatgaaagacggcgtgatgatcatcaacaccagccgcggcgggttgattgactctcaggcggcgattgaagcgctgaaaacccagaaaattggcgcgctggggatggacgtgtatgagaacgaacgcgatctgttctttgaagacaagtctaacgacgtcattcaggacgacgtgttccgtcgcctgtccgcctgccataacgtgctctttaccgggcatcaggcgttcctgaccgctgaggcactgatcagcatttccgaaaccacgctggggaatttacagcagctggaaaacggtgaagcgtgtcctaacgagatagtctaa

SEQ ID NO: 22 - DNA, 2283 bp, Enterobacter cloacae
  source 1..2283: mol_type = genomic DNA
atgtccgagcttaatgaaaagttagccacagcctgggaaggttttgcgaaaggtgactggcagaatgaagtaaacgtacgtgacttcattcagaaaaactataccccgtatgaaggtgacgaatccttcctggctggcgcaactgacgcgacaaccaagctgtgggacagcgtaatggaaggcgtaaaactggaaaaccgcactcacgcgccagttgattttgacacctccgttgcttccaccatcacttctcacgatgctggctacatcaacaaagcccttgagaaaatcgttggtctgcagactgaagcaccactgaaacgtgcgatcatcccgttcggtggtatcaaaatggttgaaggttcctgcaaagcgtacaaccgcgagctggacccaatgctgaaaaaaatcttcaccgaataccgcaaaacccacaaccagggtgtattcgatgtttacaccaaagacattctgaactgccgtaaatctggcgttctgactggcctgccagatgcctacggccgtggtcgtatcatcggtgactaccgtcgcgttgcgctgtacggtatcgacttcctgatgaaagacaaatacgcgcagttcgtttccctgcaatccgatctggaaaacggcgtaaacctggaagcgactatccgtctgcgtgaagagatcgctgaacagcaccgtgcgctgggtcagatcaaagagatggctgctaaatatggctgcgatatctctggtcctgcaactaacgctcaggaagctatccagtggacctacttcggctacctggccgcagttaagtctcagaacggtgcagcaatgtccttcggtcgcgtatccaccttcctggatgcgtacatcgaacgtgacctgaaagcaggtaaaatcaccgagcaagacgctcaggaaatgattgaccacctggtcatgaaactgcgtatggttcgcttcctgcgtactcctgagtatgatgaactgttctctggtgacccaatctgggcaaccgaatctatcggtggtatgggcgtagatggccgtactctggttaccaaaaacagcttccgcttcctgaacaccctgtacaccatgggtccttctccggagccaaacatcaccgttctgtggtctgaaaaactgcctctgaacttcaagaaattcgccgctaaagtgtccatcgacacctcttctctgcagtatgagaacgatgacctgatgcgtccggacttcaacaacgacgactacgctatcgcatgctgcgtaagcccaatggttgttggtaaacaaatgcagttcttcggtgcgcgtgcaaacctggcgaaaaccatgctgtacgctatcaacggcggcgttgatgaaaaactgaaaatgcaggttggtcctaagtctgaaccaatcaaaggcgatgtgctgaactatgacgaagtcatggaacgcatggatcacttcatggactggctggcgaaacagtacgtgactgcactgaacgttatccactacatgcacgacaaatacagctatgaagcttcgctgatggcgctgcatgaccgtgacgttatccgtaccatggcgtgtggtatcgcaggtctgtccgttgctgctgactccctgtctgcaatcaaatatgcgaaagttaaaccaattcgtgacgaagacggtctggctatcgacttcgaaatcgaaggcgaatacccgcagtttggtaacaacgacgctcgcgttgatgacatggcggttgacctggttgaacgtttcatgaagaaaattcagaaactgaccacctaccgtaacgctatcccgactcagtctgttctgaccatcacctctaacgttgtgtatggtaagaaaaccggtaacaccccagacggtcgtcgtgctggcgcgccatttggcccaggtgctaacccaatgcacggtcgtgaccagaaaggtgctgttgcctctctgacctccgttgctaaactgccgtttgcttacgcgaaagatggtatctcttacaccttctctatcgtgccaaacgcgctgggtaaagacgacgaagtgcgtaaaaccaacctcgcgggtctgatggatggttacttccaccacgaagcgtccatcgaaggtggtcagcacctgaacgtgaacgtcatgaaccgtgaaatgctgctcgacgcgatggaacaccctgagaaatatcctcagctgaccatccgtgtatctggatacgcagtacgttttaactccctgacgaaagaacagcagcaggacgttatcacccgtactttcactcagtccatgtaa

SEQ ID NO: 23 - DNA, 1002 bp, Enterobacter cloacae
  source 1..1002: mol_type = genomic DNA
atgtcagttaaagccattgccgttaacccagaaaatcccgctgctttcattgaaatttcccaacccatgccgcagcctggcgagcacgatctgttggtggaagtgaaggcagtctcaattaacccggttgataccaaagtccacgcgagtattgctaaaaatggcctcaaggatccgcgcattctcggctgggatgccagcgggatcgtcaaagcggttggggcaagcgtcaccggcttcaaaccgggcgacgaagtatggtacgcgggcgacattacccgtccgggcagtaatgccacgcaccagctgattgatgcacgcattgtcggacacaaaccgataagccttggctgggatgctgccgccgcattgccgctgaccgcgctcaccgcctgggaagggttgtttgagcgcctgaacattcaggatgccggatcggacaaaacgctgctgatcatcggcggtgccggcggcgtaggctccctggcgatcccgtttgcaaaacacaacagcaaagtcagagtgatcgccaccgcgtcgcgggaagattctgcccggtggtgtcgcgatcgcggcgcggatctggtggtgaattatcgcgatctgcaaggtgaactggccaaagagggcatcacctttgtggattacattttcatcctcaatgataccgatggccactgggatgcggccagcgaactgattgcaccgcaggggcatatctgctccatcgtcgaaaatgcgcatccgctgaaccaggacaagctgaaatccaaatccgccgccctgcactgggaatttatgtacacccgcagcatgtaccagactgccgatatggcgcgtcagggtgagatcttaaacgaggtggcgaagctggtggataacggcgtggtggagagttcgctcagcgaaacgctgcacgggctgagcgtggaaagcattaccgaggcgcaccgcaaagtgctggacggacatatgcgcgggaaagtggtggttgcgttctga

SEQ ID NO: 24 - DNA, 780 bp, Enterobacter cloacae
  source 1..780: mol_type = genomic DNA
atgatgcactcatctgcctgcgactgtgaggccagtttatgcgagaccctgcgcgggttctccgctaagcatcctgacagcgtgatctatcagacatcgctaatgagcgccctgctaagcggcgtctacgagggggacaccaccatcgccgatctgctggcgcacggtgattttggtctgggcaccttcaacgagctggacggcgaaatgattgccttcagcagccaggtgtaccagctgcgcgccgacggcagcgcacgcgccgcgaagccagagcagaaaacgccgttcgcggtgatgacctggttccagccgcagtaccgtaaaacctttgatgcgccggtcagtcgtcagcagatccacgacgtgatcgaccagcaaatcccctccgataacctgttctgcgcgctgcgcatcgacggcaacttccgccacgcccacacccgtaccgtaccgcgtcagacgccgccataccgcgcgatgaccgacgtgctggacgaccagccggtgttccgcttcaaccagcgcgaaggggtgctggtcgggttccgcacgccgcagcatatgcagggcattaacgtggccggctatcacgaacatttcattaccgacgaccgtcagggcgggggacatctgctcgactatcagttggagagcggcgtgctcacctttggcgaaatacacaagctgatgatcgatctgccagccgacagcgcgtttttacaggccaaccttcaccccagcaatcttgatgcagcgatccgttccgtcgaaaactaa

SEQ ID NO: 25 - DNA, 771 bp, Enterobacter cloacae
  source 1..771: mol_type = genomic DNA
atgcaaaaagttgctctcgtaaccggctcaggccaggggattggtaaagcgattgcgctgcgcctggtgaaagatggttttgccgtcgctatcgccgactacaacgatgagacggcgagagccgtcgccgatgaaatcatccgtaacggcggcaacgccgtcgccgtgaaggtggatgtctctgatcgcgaccaggtgtttgcggcggtggagaaagcccgcaccgcgctgggcggtttcaacgtgattgtgaataacgccgggattgcgccatctacgcccatcgaatccatcacgccggagattgtcgacaaggtctacaacatcaacgtgaaaggggtgatctggggtatgcaggccgcgattgacgcgtttcgcaaagaggggcacggcggcaaaatcatcaacgcctgctcccaggcgggccacaccggtaacccggaactggcggtgtacagctcaagtaaattcgcagtgcgcggcttaacccagaccgccgctcgggacctcgcgccgctggggatcaccgttaacgcctactgcccgggtatcgtgaaaacgccgatgtgggcggagattgaccgtcaggtgtccgaggcggcaggtaaaccgcttggttatggcaccgaaacctttgccaaacgcattacgcttggccgcctgtccgaaccggaagatgtcgctgcctgtgtttcatacctcgccgggccagactctgactacatgactggtcagtcgctcttgattgatggtgggatggtgttcaactaa

SEQ ID NO: 26 - DNA, 1104 bp, Enterobacter cloacae
  source 1..1104: mol_type = genomic DNA
atggaccgtatcattcaatcgccggggaaatatatccagggcgctgatgttctcacccgtctcggcgactatctgaaacctctggctacacgctggctggttgttggcgataaatttgtgctgggttttgccgaagagaccctgcgccagagttttaaaaatgcagagctacatgccgaaatcgcgccgtttggcggcgaatgttcgcagaatgagatcgatcgtctgaaaaaactggccgacagtgcggactgcatggcggtgctgggcattggcggcggtaaaacgctggataccgccaaagcgctggcacatgttatggacgtgccggtcgctatcgccccgactatcgcctccaccgatgccccgtgcagcgccctttctgtgatttacaccgacagcggcgaatttgaacgctatctgatgctgccgcataaccccaatatagtgattgtcgacaccaaagtggtggcaggtgcgccgccgcgtctgctggccgccgggattggcgatgcccttgccacctggtttgaagcgcgtgcctgttcccgcagtggcgcgaccaccatggcgggcggcaagtgtacgcaggccgcgttagcgctcgccgagctgtgctacaacacgctgattgaagagggagaaaaagcaatgctggccgccgaacagcacgtagtgaccccggcgctggagcgcattatcgaagccaatacctatctgagcggcgtagggtttgaaagcggcgggctggcggcagcgcatgctattcacaacggaatgacggcggtgccggatgcgcatcacttctatcacggtgaaaaagtggcgtttggtacgctgacgcaactggtgctggaaaacgcaccggttgaggagatcgaaaccgtcgcggcgctttgccacagcgttggattgccgatcacgctggcgcagctgaacatcaaagaagacattccggccaaaatgcgcctgattgccgaagcgtcttgcgctgagggagagaccattcacaacatgccgggtggcgtgtcgccggatcaggtttatgcggcgctgctggtcgccgaccagtatgggcagcgtttcctgcaggagtgggagtaa

SEQ ID NO: 27 - DNA, 1722 bp, Bacillus licheniformis
  source 1..1722: mol_type = genomic DNA
atggcaaacaaaaccgcaggaaaaatagctgcagaactgctgaaggaatggaacattgatcatgtttacggcatgccgggcgacagcgtcaacgaatttatcgatgaattgcggcatgaagaaaacagcctccgcttcattcaagtgcgtcatgaagaaaccgctgctctcgctgccgcagccgatgcaaagctgacaggcaaaatcggcgtctgcctttcgatagcgggcccgggcgccgttcatctgctgaacggcttgtatgatgcgaaagctgacggggcgcctgtgcttgcgattacaggacaggtgtcttcggatgaaatcgggagagattattttcaggagatcggtcttgaacgaatgtttgaggatgtggctctctttaatcagcaggtgcattcggctgaagccctgccagatcttttgaatcaggcgatccgtaccgcatacagccaaaaaggggccgccgttctgagcgtatccgatgatctgtttgcagaaaagattaagcggaagccggtttatacgtctgcgctttatatcgaaggcgatcttgaaccgaagaaaagccagctgatgcagtgtgcacaattgattaaccaggcaaaaaaaccggtgattttagcgggacggggaatgaaatcggcgagagacgagctgctcgagtttgcagataaagcggccgctccgatcattgtcacactcccagcgaaaggggtcgtacctgatcggcacccgcatatgctcgggaacctcggacacatcggcacaaagcctgcatatgaagcgatggaggaaagcgatctgttgatcatgctggggacttcatttccgtaccgcgattatctgccggaagatgccccggctattcagcttgacaacaatccggcgaaaatcggcaaacggtatcctgtcaccgccggccttgtctgcgatgcgaaaaaagggctttttgaactgacgaaaacaattgaacggaaatcgaaccgcgcatttttggaaagctgtatacaacatatgaggaagtggcgttatgaagttgaaaaagacgaacaggtagcgacagagccgctgaagcctcagcaggtgattgcaagacttgaggacgcggtcgcagatgatgcggttctgtccgttgacgtcggaaatgtgacggtttggacggcgcgccattttaatatgaccaatcaggattttttgatatcaagctggcttggcacgatgggatgcggtcttccaggcgcgatttctgcaaagctttcccatccggaacgccaggttgtagccgtgtgcggagacggcggtttcagcatgtctatgcacgattttccgactgctgtcaaatacgaactgcccattgttgttgtgattttaaataatcagaacttgggaatgattcaatatgaacagcaggaaaaagggcatgttaattatgcgacggcgctcgaaaacgtcgattatgccaagtttgccgaagcctgcggcgggaagggcttcagcgtgacaaagcacgaagagcttattcctgctttgaaaagcgcctttcattcacaaaagccgtcgattatcgatgtggcaattgaagacgaaccgcctcttccgggaaaaatatcctatacacaagctgtaaactacagcaaatatatgatcaaaaaactagttgagaaaaaagagctcgacttgccgccgctgaaaaaaagtttaaaaaggattttttag

SEQ ID NO: 28 - DNA, 972 bp, Bacillus licheniformis
  source 1..972: mol_type = genomic DNA
gtggcagatctatttacaacagtgcagggaaaagtagcaggaaaaggagttaaaatcgtatttccggaaggtttggacgagcgtattcttacagctgtcaacaaattggccggaaacaacgtgttaaagccgatccttgtaggaaacgagcaggaaatcaaagataaagcaaacgggttgaatttaacgctggatggcgttgacatatacgatcctcatacatatgaagggatggaagaccttgttcaggctttcgttgaacgccgcaaaggcaaagcgacggaagagcaggcccgccaaatccttttagacgaaaactactttggaacgatgcttgtttataaagggcttgccgacggccttgtcagcggtgcggctcattcaacagcggatacggttcgcccggcgctgcaaattattaaaacaaaagaaggcgtgaagaaaacatcaggcgtctttatcatggcgcgcggcgatgagcagtacgtattcgcagactgtgcgatcaacattgcgcctgacagccaggaccttgcggaaatcgcaatcgaaagtgcgaacacagcgaaaatgttcgacatcgaacctcgtgtcgccatgctcagcttttcaacaaaaggttcggcaaaatcagacgaaactgaaaaagtagccaacgcggttgcaatcgcaaaagaaaaagcgcctgagcttacacttgacggcgaattccaatttgacgcggcattcgttccgtctgtcgctgagaaaaaagcgccggactccgtcattaaaggagatgcaaacgtatttgttttcccaagccttgaagcggggaatatcggctataaaatcgctcagcgcctcggcaactttgaagcggtcggtccgattctgcaagggctgaacatgcctgtaaacgacctgtccagagggtgcaacgctgaggacgtttataatcttgcgctgattacggcggctcaagctctgtaa

SEQ ID NO: 29 - DNA, 1389 bp, Bacillus licheniformis
  source 1..1389: mol_type = genomic DNA
gtggactacagaattgaaaaagatacgatgggtgaagtcaaggttcccgcggataagttttggggagcccaaacccaaagaagcaaggagaactttaaaatcggttccgagaaaatgccgcaggaaattgtgtatgcgtttgcaatcttgaaaagaagcgctgcgattgccaatgaaaagcttggaaatcttgaggcggaaaaaaaggaagcgattgtttccgtatgcgatgatattttagaaggaaagtatgacagccattttccgctggtcgtctggcagaccggcagcggaacacagagcaacatgaatatgaatgaagttgtggcaaacaggggaacggcttatttgcaggaaaaaggatcaagcctgaaaatccacccgaatgatgatgtcaaccgcagccaaagctctaacgacacattcccgacagctatgcacgtggcagctgtcctcgcgattcacgataaactgattccggcgattgaccggttgagaagcacgctgcatgaaaaagctgaagctttcaaagacattgtcaaaatcggccgcactcatttgcaggatgcgacaccgctgacattgggacaggaaatcagcggctgggtatacatgctggaccgctctaaagagatgattcttgaagctgctgaaaaaatccgcgatcttgccatcggcggaactgccgtcggaacaggcattaatgcccaccctgaattcggcagcaccgttgccgaagagatcagcgctctgaccgggcagacattcaaaagctcgcctaataagtttcatgcgctgacaagccatgatgaactgacacacgtgcatggggcgcttaaagcgcttgcggcagatctcatgaaaatcgccaatgatgtcagatggcttgcgagcggacctcgctgcggaatcggcgagctgcttatcccggaaaatgagccgggaagctcgatcatgcctggaaaagtaaaccctacgcaaagcgaagcgctgacaatggtcgcatcccagatcatggggaatgacgcaacgatcggctttgccgcgagccaggggaactttgaactgaacgtctttaaacctgtgattatttacaacttcctccaatccgttcagcttcttgccgacggcatgaattcattccatgacaagtgtgctgtaggaatcgaacctaatcgggaaacaattgagaagaacctgaccaactctctcatgctggttacggctctaaacccgcatatcggatacgaaaatgctgcgaaaatcgcgaagctggctcataaagaggggctgacgctgaaagaagcggctctcaagctgaaccttttgacagaggagcagttcgacgaagttgttaaacctgaagaaatggtgaaaccgaaagcataa

SEQ ID NO: 30 - DNA, 960 bp, Bacillus licheniformis
  source 1..960: mol_type = genomic DNA
atgagaaaccaaaaagtcaatcgtacagttttgatcggtgcgggttttgttggaagcagctatgcatttacgttaattaaccaaggtatcacagacgaacttgtgatcattgatttaaataaagacaaagcaatgggcgatgttatggatctcaaccacggtaaagcatttgctccgcatcctgtgaaaacatggtacggcacatatgatgactgcaaagaagctgatattgtctgcgtatgcgccggagcgaaccaaaagccgggtgaaacccgccttgatctagtcgaaaagaacttgaaaatttttaaaggaatcatcggagaagtcatggccagcggttttgacggcatcttcctcgtcgctacaaaccctgtcgacatcttgacttatgcgacttggaaattcagcggccttccaaaagaacgcgtcatcggaagcggcacaacgcttgacactgcccgtttccgctacttgctcagcgagtatttcggtgtcgctgcccacaatgcgcacggctatatcattggtgaacacggagatacggagctgcctgtctggagccatgcgaatatcggcggagttcccgtcagcgacttgctgaaaagaaatgagaaatacaaagcggaagatctagatgaactgtttgacaatgtaaaaaacgccgcttaccacattattgagaaaaaaggcgcgacatactacggagttgccatgagcctcgcacgtattacgaaagcgatttatcgaaatgaagaagcaattttgaccgtgagcgctcatcttgatggtgaatttggagaaaatgatgtttatatcggagttccggcggttgtcggccgatgcggagcgagggaaattgttgagctcgatctgaacgaaaaggaaaaacaacaattcaagcatagcgcaggtgtgttaaaagcaattttaaaaccgcattttgaactgcaggcgtaa

SEQ ID NO: 31 - DNA, 2226 bp, Bacillus licheniformis
  source 1..2226: mol_type = genomic DNA
atggaacaatggaaaggtttcaccacaaacgtttggcaaaaagaagtcaatgtccgcgattttattctctcgaactttgagccatatcaaggtgacgaatcgtttctcgaacctccgacggaagctacatcggcattatgggatcatgtaatggatttgacaaaaaaagagcgtgaaaacggaggcgtccttgatatggatacagagatcgtctcaacgatcacctcgcacggtcccggatatttgaacaaagacctggaaaaagtcgtcggcgttcaaaccgatgagccgtttaaacggtcgcttcagcctttcggcggcatccgaatggcaaagcaggcatgcgaatcctatggttttaaactgaatgaagaagtggaaaggatctttaccgattaccgcaaaactcataaccaaggcgtgtttgacgcatatacggacgaaatgaagctcgcccgaaaagtcggaatcattaccggactgcctgatgcttacgggcgcgggcgcatcatcggtgattaccggagagtggcgctttacggcgtggatttcttgatcgatgaaaagaaaaaagatgcggccggcacctctcgggtgatgtctgaagaaaacatccgccttcgtgaagaactgtcagaacaaatccgagcattgaacgaacttaaagcgcttgcaaaaagctatgggtttgacatttccaagcctgcggcgaatgcaagagaagcatttcaatggctgtattttgcctatttggctgccattaaagagcaaaacggagcagcaatgagccttggccgcgtgtccacgttccttgatatttacatcgaaagagatttgaaaacgggcgtattaacagagcgtgaagcccaagagcttgtcgaccatttcgtcatgaagctgcgtttggtcaaattcgcgcgcacacctgactacaatgaactgttcagcggcgatccgacgtgggtgacagaatcaatcggcggaatggcgcacgacggacgcgccctggtgacgaaaaactcgttccgtttcctgcatacgcttgacaatttaggcccggcgcctgaaccgaatttaaccgttctttggtctgtcagactgccgcaaaagtttaaaaactactgtgccaaaatgtcgattaaaacaagctcgatccaatacgaaaatgacgatatcatgcgtccagaatacggtgatgactacggaatcgcctgctgtgtatcggcaatggcaatcggcaaacaaatgcagttcttcggagcacgcgccaacttggcgaaagctcttttatatgcgattaacggcggaaaagacgaaaagcataaaatgcaagtcggtccggaaatgccgccggttgcttccgacgtgctggactatgacgaagtgatgcataaattcgatcagacgatggaatggctcgcaggcttgtacatcaacacgctcaatgtcattcactacatgcatgataaatattgctatgaaagaattgaaatggccctgcacgacacggaaattttgcggacgatggccactgggatcgccggcttgagtgttgtcgccgattcattaagcgctgtcaaatatgccaaagtcagcgtggtccgcgatgaaaacggcattgcggtcgattttgaaacagaaggcgactttcctaagtacggaaataacgatgaccgcgtcgacgcgatcgccgttgacattgtcaagcgctttatgaaaaaactgcgcaagcatcagacatatcgccagtccgttcagaccatgtcaattttaacgatcacgtcaaacgtcgtttacggcaagaaaaccggaaatacgccggatggacgccgcgcgggagaaccgtttgctccaggtgcgaatccgatgcacggccgcgatactaaagggacgcttgcatcgctgtcttcagtggcaaagctgccttacagctatgcgctcgacggcatttccaacaccttttcaatcgtcccgaaagcgcttggcaaagacgaagagagccgcgccgccaatttgtcaagcatccttgacggatatgccgcaaaaacaggacatcacttaaatgtaaacgtatttaacagagagacactgctcgacgccatggaacatccagaggaatatccgcagttaacgattcgcgtctcaggctatgcggtcaactttattaagctgacgaaagaacagcagttagacgtcatcagcagaaccttccatgaatcgatgtag

SEQ ID NO: 32 - DNA, 2604 bp, Bacillus licheniformis
  source 1..2604: mol_type = genomic DNA
atggcaatcgaagaaaagaacatgaaacaaaagcaaaatgcctcaaacatgattgatcagctagtcgaaaaaggcctaaaggcattggaagaattccgcagctttgatcaggagcagattgatgagatcgtcaaacagatggcgcttgccggccttgatcagcacatgcctctcgccaaacttgccgtcgaagaaacgaagcgcggcgtttatgaagataaaatcattaaaaacatgtttgccacagagtatgtctaccatcatatcaaatacgataaaacagtgggcatcatcaatgaaaacgaacacgacggagtcattgaaatcgcggagcctgtcggtgtcatcgccggcgtgacgccggtgaccaaccctacttccaccaccatgttcaagtccctgatatcaattaaaacaagaaatccaatcgtgtttgctttccatccatccgcccaaaaatgcagccgcgaagcggcgagaatccttcgggacgcagccgtcaaagcaggagcacccgacaactgcatccaatggatcgagacgccttctctggatgcaacgcaagccctaatgacccacccgaacgtttccctgatcctcgcaacaggaggcgcaggcatggtgaaatccgcttacagctccggaaaaccggcccttggcgtcggacctggaaacgttccgtgctacatcgagaagtctgccaacctgaagcaagccgtcaatgatctgatcttatccaaaacatttgacaacggcatgatttgcgcatcagagcaggccgtcatcatagacaaaggcatctactcagacgtcaaagccgaaatgacaagaaacaactgctatttcttaaataaaaccgaaaaatcaaaggtcgaaaaattggtcatcaatgaaaatacttgcgcggtaaacgctgatatcgtcggcatgccggcattcaaaattgctgaaatggccggtatcaaagtgccgcaagatacaaaaatcctcatcgccgaattagaaggtgtaggtccggatgacccgctgtccagagaaaaactgagccctgttttagcgtgctacaaagtcagcgggcttgaagaaggattaaaacgcgccgaagagatgctggcgttcgggggaaccggccactcggcggtgattcatacgaacgatcaagaagccgttaaagaattcggcttaaggatgaaggccggccggatcattgtcaatgcaccgtcgtcacaaggagcgatcggcgacatttacaacgcctatatgccttctttaacgctcggctgcggcacatatggcgggaactccgtctcgtcaaacgtaggggccgttcatttaatcaacacgaaaaaagtggcgaaaaggaatgtcaatatgcagtggtttaaagtgcctccaaaaatctattttgaaaaacacgcaacccaatatttagcgaaaatgcctgacatctcaaaagcgtttattgtgactgatccgggaatggtgaaactcggatatgtagaccgcgcccttcactacttaagaaggcgcccggattacgttcattgcgaaattttttctgatgtcgagcctgatccttcaatcgaaacggtgatgaacggcgtcgacatgatggccaaattccagccggatgtcatcatcgccctcggcggaggttcggcaatggatgccgcaaaaggaatgtggatgttctacgagcaccctgatgctgaattctttggcttaaagcaaaaatttctggacatccgcaagcgcatcgtcaaatacccgaaactgggaggaaaagcaaaattcgtcgcaatcccgacgacatcaggcacaggttctgaagtcacttcattctctgtcattacagacaaagaaacgaatacaaagtacccgctggccgattatgaattaacaccggatgtcgcgatcattgacccgcaatttgtcatgaccgtgccgaaacatatcacggccgacacaggaatggacgtattgacacacgcaattgaatcatacgtatcgtgcatggccaacgactatactgacggcttggccatgaaggccatccagctgatcttcgaatatctgccaagagcatacaaaaacggcagcgacgagctggcccgcgaaaaagtgcataatgcatcaacgattgccggaatggcattttccaatgccttcctcggcatcaaccacagcttagcccataagcttggcgccgagtttcagatcgctcatggacgggcaaacgccatccttctgccgcacgtgatccgctataatgcagcgaagccgaaaaagttcacggcatttcctaaatacagccacttcatagccgaccagcgctacgcggagattgccagaacgctcggcctgcctgcaaaaacaactgcagaaggtgttgaaagcctcatccaggagatcatctcgcttgccaaagagcttaaaattccgatgagcattaaacaaaacggcgtggatgccgccgcattcgaaagcaaagtagatctcatggccgagcgcgcctttgaagatcagtgcacgacagccaatcctaagctgccgctcgtttcagagctggctgagatttaccgttccgcgtataaaggagtttag

SEQ ID NO: 33 - DNA, 762 bp, Bacillus licheniformis
  source 1..762: mol_type = genomic DNA
atgaaaagtgcaagcaaacaaaaaataattcagcccgttgataagaacctcgatcaagtctatcaggtctcaacgatggtatctttattggacggaatttacgacggggatttttatatgtccgaagcgaaggagcacggagacttcgggatcggaacgttcaaccggctcgacggcgagctgatcggttttgacggtgagttttaccgtctccgttccgatggaaaagcctacccagttcaaggaagcgattgttctccattttgctcgctggctttcttccggccggatatctatcacgaaatcaagcagcggatgccgcttgaggcgttcgaagaagaaatgaaacggatcatgccgagtgaaaacctgttttacgcgattcgcatggacggaacctttaagaaagtcaaaacgagaacagttgaacttcaggaaaaaccgtatgtgccgatggttgatgcggtaaaatcacagccgatctttgattttaatgatattacggggacgatcgtcggcttttggacaccgcaatatgccaacggaatcgcagtttccggcttccatcttcactttatagatgaagaccgcaatgtcggcggacacgttttcgattatgaaatcgaagaatgcacggtgcaaatttctcaaaaactcaatatgaacctcagattgccgaatacgcaagatttctttcaagcggatttcaataaacacgatcttgcagccggaattgaagcggccgaaggcaatcccgagtaa

SEQ ID NO: 34 - DNA, 783 bp, Bacillus licheniformis
  source 1..783: mol_type = genomic DNA
atgagtaaagtatctggaaaaattgcttttgttactggcggcggtcaaggaattggagaagcaatctgcaaacgattggcagaggacggattcgcagttgcagttgccgattataatgtagaaactgcaacacaagttgctgaggacatcaataagcttaacggcaaagcaattgcggttaaagtggatgttgctgatcgcgatgatgtttttaaagctgtcgatgaaacagtaaaacgtcttggcggtcttgatgtggtgattaataatgcgggtcttggaccaaccacccctattgaaagcattacatatgaagattatcggaaagtctatgatgttaacgttggcggtacttattggggaatacaagcagctgtaaaagcctttaaagaacttggacacggcgggaaaatcattaatgcatcttctcaagccggccaagtcggcaacccgggcttagcggtttacggaggaacaaagttcgctgttcgcgggattacccaaactgcggcaaaagatctagctgaattaggtattactgtaaacgccttttgtccgggtatcgttaaaactcctatgatgatggggattgcacagcaaaccgctgatgaagcaggcaagccgtttgaatggggcatggaacaattcgctaaaaatattgcattaaaacgcttatccgagccggaagatgtagcagcatgcgtttcttaccttgcagggccagattcagattatatgactggtcaagctcttatcattgatggcggaatggtatttaattaa

SEQ ID NO: 35 - DNA, 1104 bp, Bacillus licheniformis
  source 1..1104: mol_type = genomic DNA
atgtcaaaatcagtaaaatcagtcacatcacctaaaaaatttattacaggaaaacgactgctggagaacttgaacgactacattgaagattttggcgacaacgcatatatcatttgcgatgaattcattttggaacgcgctcaaaaagaagcggggaattcgattcagaaagccggcaatcaagccgtttttgaaaaattcaattacgaatgcacacaggaagaaatcgatcgcaaccgggagcttgcacgcaatgcaggcgctaatatcatcgttgggatcggaggcggtaaaacgcttgataccgcaaaagccaccgcttattacgagaagctgccggttgtgattttcccgacaattgcttctacggatgctccatgtacggcccttgccgtcatttataaacacgacggatcgtttgaccgctatctgtttttgccgacgaacccagatgtcgttcttgcggactctgagattttggcatccgcgccgccgcgctttttcgcagccggtatcggtgacgccttggcgacgtattttgaagcgcgtgcctgctttaaagcaaacggcgataacctcgtgctgatgaagccttcaacaactggattgggacttgcccgtctttgctatgatacgctgttggaaaacggtgtgaaagcgatgcaggcggttaagcacggcgtttccacacgagcggtcgaagatacaatcgaggcgaccatctatttaagcggcgtcggtgccgaatcaggcggtcttgccgccgcacacgcgatccacaacggaatgacagccgttccttctctgcacagggctcagcacggcgaaaaagtcacgttcggccttttggcgcagcttgttcttgaaaacgcgccggccgaagaattggagaccgttattgactttatcaaaggcgtcggtcttccgttgacattaaaagacctcggagtcgacgaatttgtcgaagaagaatggcgccaagtcgctcaaagcgcttgcgcggaaggcgacacaatgggcaacatgccgttcccagtcacccctgacgacgtctacaatgcgatcgtcgccgccaacgcgattgcagaatcttatcacgattaa

SEQ ID NO: 36 - DNA, 29 bp, synthetic construct
  source 1..29: mol_type = other DNA
ccggaattcacagaccgtggcggcataca

SEQ ID NO: 37 - DNA, 40 bp, synthetic construct
  source 1..40: mol_type = other DNA
cgcttaccgttcatctgcaaagctgggccagctttttcag

SEQ ID NO: 38 - DNA, 40 bp, synthetic construct
  source 1..40: mol_type = other DNA
ctgaaaaagctggcccagctttgcagatgaacggtaagcg

SEQ ID NO: 39 - DNA, 28 bp, synthetic construct
  source 1..28: mol_type = other DNA
cgcggatccttaccttagccagttagtt

SEQ ID NO: 40 - DNA, 32 bp, synthetic construct
  source 1..32: mol_type = other DNA
ccggaattcactggcggtaacgaaagaggata

SEQ ID NO: 41 - DNA, 38 bp, synthetic construct
  source 1..38: mol_type = other DNA
taaacctgttccggcagcacgaagctgctgcgagtcag

SEQ ID NO: 42 - DNA, 38 bp, synthetic construct
  source 1..38: mol_type = other DNA
ctgactcgcagcagcttcgtgctgccggaacaggttta

SEQ ID NO: 43 - DNA, 31 bp, synthetic construct
  source 1..31: mol_type = other DNA
tgctctagattatgcttgctgctgggacgac

SEQ ID NO: 44 - DNA, 29 bp, synthetic construct
  source 1..29: mol_type = other DNA
ccggaattcataccgttgctgctgaaggg

SEQ ID NO: 45 - DNA, 40 bp, synthetic construct
  source 1..40: mol_type = other DNA
cttcgcccagttctcgttactggtattgtagcgatacacg

SEQ ID NO: 46 - DNA, 40 bp, synthetic construct
  misc_feature 1: note = 1
  source 1..40: mol_type = other DNA
cgtgtatcgctacaataccagtaacgagaactgggcgaag

SEQ ID NO: 47 - DNA, 27 bp, synthetic construct
  source 1..27: mol_type = other DNA
cgcggatcctcagccattcgtcgtctc

SEQ ID NO: 48 - DNA, 28 bp, synthetic construct
  source 1..28: mol_type = other DNA
ccggaattctacgaaacagtacgacaag

SEQ ID NO: 49 - DNA, 39 bp, synthetic construct
  source 1..39: mol_type = other DNA
gaatcgatgagcgcgccgcaccgcttccggcgagtaggc

SEQ ID NO: 50 - DNA, 39 bp, synthetic construct; identical to SEQ ID NO: 49
  source 1..39: mol_type = other DNA

SEQ ID NO: 51 - DNA, 29 bp, synthetic construct
  source 1..29: mol_type = other DNA
cgcggatcctgacgcaggttgtcgagggt

SEQ ID NO: 52 - DNA, 29 bp, synthetic construct
  source 1..29: mol_type = other DNA
ccggaattcttaatgaaaagttagccaca

SEQ ID NO: 53 - DNA, 40 bp, synthetic construct
  source 1..40: mol_type = other DNA
cgcgagagtcgttgttaccagacggtagtcaccgatgata

SEQ ID NO: 54 - DNA, 40 bp, synthetic construct
  source 1..40: mol_type = other DNA
tatcatcggtgactaccgtctggtaacaacgactctcgcg

SEQ ID NO: 55 - DNA, 29 bp, synthetic construct
  source 1..29: mol_type = other DNA
tgctctagattacatggtctgagtgaagg

SEQ ID NO: 56 - DNA, 50 bp, synthetic construct
  source 1..50: mol_type = other DNA
aacagctatgacatgattacgaattcgccgctattgtggcacgttcgacc

SEQ ID NO: 57 - DNA, 38 bp, synthetic construct
  source 1..38: mol_type = other DNA
gcttttctttgtcactcataagacttttctccagtgat

SEQ ID NO: 58 - DNA, 38 bp, synthetic construct
  source 1..38: mol_type = other DNA
atcactggagaaaagtcttatgagtgacaaagaaaagc

SEQ ID NO: 59 - DNA, 40 bp, synthetic construct
  source 1..40: mol_type = other DNA
gcacaaaagggaaaggaatattagtgattgtctttgggta

SEQ ID NO: 60 - DNA, 40 bp, synthetic construct
  source 1..40: mol_type = other DNA
tacccaaagacaatcactaatattcctttcccttttgtgc

SEQ ID NO: 61 - DNA, 51 bp, synthetic construct
  source 1..51: mol_type = other DNA
gcctgcaggtcgactctagaggatccgttactgtcggcgtgtagtagcaat

SEQ ID NO: 62 - DNA, 48 bp, synthetic construct
  source 1..48: mol_type = other DNA
aacagctatgacatgattacgaattctcaaacaatgattgaatcacag

SEQ ID NO: 63 - DNA, 40 bp, synthetic construct
  source 1..40: mol_type = other DNA
atatatttaaaaagttccataatgctctcctgataatgtt

SEQ ID NO: 64 - DNA, 40 bp, synthetic construct
  source 1..40: mol_type = other DNA
aacattatcaggagagcattatggaactttttaaatatat

SEQ ID NO: 65 - DNA, 40 bp, synthetic construct
  source 1..40: mol_type = other DNA
ctgactttacggctgtggaattaacgtctgcttaatacac

SEQ ID NO: 66 - DNA, 40 bp, synthetic construct
  source 1..40: mol_type = other DNA
gtgtattaagcagacgttaattccacagccgtaaagtcag

SEQ ID NO: 67 - DNA, 51 bp, synthetic construct
  source 1..51: mol_type = other DNA
gcctgcaggtcgactctagaggatccaggtggtggaccagctcgatattcc

SEQ ID NO: 68 - DNA, 48 bp, synthetic construct
  source 1..48: mol_type = other DNA
aacagctatgacatgattacgaattctcgccatattgccctcgacctg

SEQ ID NO: 69 - DNA, 40 bp, synthetic construct
  source 1..40: mol_type = other DNA
gtattgaagtagttagccattacccgcttcctcgttcaac

SEQ ID NO: 70 - DNA, 40 bp, synthetic construct
  source 1..40: mol_type = other DNA
gttgaacgaggaagcgggtaatggctaactacttcaatac

SEQ ID NO: 71 - DNA, 67 bp, synthetic construct
  source 1..67: mol_type = other DNA
gctttacgccaggacgcgcgctcctcggcaatcgcttctttaaactcagcgtaggagatatcgagac

SEQ ID NO: 72 - DNA, 67 bp, synthetic construct
  source 1..67: mol_type = other DNA
gtctcgatatctcctacgctgagtttaaagaagcgattgccgaggagcgcgcgtcctggcgtaaagc

SEQ ID NO: 73 - DNA, 36 bp, synthetic construct
  source 1..36: mol_type = other DNA
atccacgagaatctccttaacccgcaacagcaatac

SEQ ID NO: 74 - DNA, 36 bp, synthetic construct
  source 1..36: mol_type = other DNA
gtattgctgttgcgggttaaggagattctcgtggat

SEQ ID NO: 75 - DNA, 50 bp, synthetic construct
  source 1..50: mol_type = other DNA
gcctgcaggtcgactctagaggatccaggcgctgccggggcgtccctgct

SEQ ID NO: 76 - DNA, 51 bp, synthetic construct
  source 1..51: mol_type = other DNA
aattcgagctcggtacccggggatccaatatatgactgccaacggtcaatg

SEQ ID NO: 77 - DNA, 39 bp, synthetic construct
  source 1..39: mol_type = other DNA
atctcttgcgttttttgcacgtaacacctaccttcttaa

SEQ ID NO: 78 - DNA, 39 bp, synthetic construct
  source 1..39: mol_type = other DNA
ttaagaaggtaggtgttacgtgcaaaaaacgcaagagat

SEQ ID NO: 79 - DNA, 40 bp, synthetic construct
  source 1..40: mol_type = other DNA
tacgatttcagtcaataccattagaaaagattcaccagtc

SEQ ID NO: 80 - DNA, 40 bp, synthetic construct
  source 1..40: mol_type = other DNA
gactggtgaatcttttctaatggtattgactgaaatcgta

SEQ ID NO: 81 - DNA, 51 bp, synthetic construct
  source 1..51: mol_type = other DNA
gcctgcaggtcgactctagaggatccaccttctttcttacaggcgcggaac

SEQ ID NO: 82 - DNA, 46 bp, synthetic construct
  source 1..46: mol_type = other DNA
gagctcggtacccggggatccgtttgcccttcatccgctgcgcatc

SEQ ID NO: 83 - DNA, 40 bp, synthetic construct
  source 1..40: mol_type = other DNA
tcttttgttgcttttgtcaatgctggattccttctgtagt

SEQ ID NO: 84 - DNA, 40 bp, synthetic construct
  source 1..40: mol_type = other DNA
actacagaaggaatccagcattgacaaaagcaacaaaaga

SEQ ID NO: 85 - DNA, 39 bp, synthetic construct
  source 1..39: mol_type = other DNA
tgtcagagcttatttattactagagagctttcgttttca

SEQ ID NO: 86 - DNA, 39 bp, synthetic construct
  source 1..39: mol_type = other DNA
tgaaaacgaaagctctctagtaataaataagctctgaca

SEQ ID NO: 87 - DNA, 46 bp, synthetic construct
  source 1..46: mol_type = other DNA
caggtcgactctagaggatccgcgggtctttttgcgcgagctgatc

SEQ ID NO: 88 - DNA, 46 bp, synthetic construct
  source 1..46: mol_type = other DNA
gagctcggtacccggggatccgccgtattctttcctcaacgacact

SEQ ID NO: 89 - DNA, 44 bp, synthetic construct
  source 1..44: mol_type = other DNA
gtattgaagtagttagccatttctattcctcctggatgaccgtg

SEQ ID NO: 90 - DNA, 44 bp, synthetic construct
  source 1..44: mol_type = other DNA
cacggtcatccaggaggaatagaaatggctaactacttcaatac

SEQ ID NO: 91 - DNA, 45 bp, synthetic construct
  source 1..45: mol_type = other DNA
ctgctggcgggtatgtcgcgaggggttaacccgcaacagcaatac

SEQ ID NO: 92 - DNA, 45 bp, synthetic construct
  source 1..45: mol_type = other DNA
gtattgctgttgcgggttaacccctcgcgacatacccgccagcag

SEQ ID NO: 93 - DNA, 46 bp, synthetic construct
  source 1..46: mol_type = other DNA
caggtcgactctagaggatccataacgaggtcaaggtctgccaggc

SEQ ID NO: 94 - DNA, 46 bp, synthetic construct; identical to SEQ ID NO: 88
  source 1..46: mol_type = other DNA

SEQ ID NO: 95 - DNA, 44 bp, synthetic construct; identical to SEQ ID NO: 89
  source 1..44: mol_type = other DNA

SEQ ID NO: 96 - DNA, 44 bp, synthetic construct; identical to SEQ ID NO: 90
  source 1..44: mol_type = other DNA

SEQ ID NO: 97 - DNA, 45 bp, synthetic construct; identical to SEQ ID NO: 91
  source 1..45: mol_type = other DNA

SEQ ID NO: 98 - DNA, 45 bp, synthetic construct; identical to SEQ ID NO: 92
  source 1..45: mol_type = other DNA

SEQ ID NO: 99 - DNA, 46 bp, synthetic construct; identical to SEQ ID NO: 93
  source 1..46: mol_type = other DNA

SEQ ID NO: 100 - DNA, 50 bp, synthetic construct
  source 1..50: mol_type = other DNA
aacagctatgacatgattacgaattcctgtgagttaaagtttccatcccg

SEQ ID NO: 101 - DNA, 40 bp, synthetic construct
  source 1..40: mol_type = other DNA
gagttcagttttgccggcataagacttttctccagtgata

SEQ ID NO: 102 - DNA, 40 bp, synthetic construct
  misc_feature 1: note = 1
  source 1..40: mol_type = other DNA
tatcactggagaaaagtcttatgccggcaaaactgaactc

SEQ ID NO: 103 - DNA, 43 bp, synthetic construct
  source 1..43: mol_type = other DNA
gcacaaaagggaaaggaatattatgccggacgcgtaaccgcgc

SEQ ID NO: 104 - DNA, 43 bp, synthetic construct
  source 1..43: mol_type = other DNA
gcgcggttacgcgtccggcataatattcctttcccttttgtgc

SEQ ID NO: 105 - DNA, 51 bp, synthetic construct
  source 1..51: mol_type = other DNA
gcctgcaggtcgactctagaggatcctgtagtagcaatgatgaacctgttc

SEQ ID NO: 106 - DNA, 50 bp, synthetic construct
  source 1..50: mol_type = other DNA
aacagctatgacatgattacgaattcggcacgttcgacctgtgagttaaa

SEQ ID NO: 107 - DNA, 40 bp, synthetic construct
  source 1..40: mol_type = other DNA
gcggaacggtacttaggcataagacttttctccagtgata

SEQ ID NO: 108 - DNA, 40 bp, synthetic construct
  source 1..40: mol_type = other DNA
tatcactggagaaaagtcttatgcctaagtaccgttccgc

SEQ ID NO: 109 - DNA, 38 bp, synthetic construct
  source 1..38: mol_type = other DNA
gcacaaaagggaaaggaatattaaccccccagtttcga

SEQ ID NO: 110 - DNA, 38 bp, synthetic construct
  source 1..38: mol_type = other DNA
tcgaaactggggggttaatattcctttcccttttgtgc

SEQ ID NO: 111 - DNA, 51 bp, synthetic construct
  source 1..51: mol_type = other DNA
gcctgcaggtcgactctagaggatcctcggcgtgtagtagcaatgatgaac

SEQ ID NO: 112 - DNA, 29 bp, synthetic construct
  source 1..29: mol_type = other DNA
ccggaattctcgaagctgtcatgatcctg

SEQ ID NO: 113 - DNA, 40 bp, synthetic construct
  source 1..40: mol_type = other DNA
ggggagagggggtactgccggggtaattctccgatttcag

SEQ ID NO: 114 - DNA, 40 bp, synthetic construct
  source 1..40: mol_type = other DNA
ctgaaatcggagaattaccccggcagtaccccctctcccc

SEQ ID NO: 115 - DNA, 34 bp, synthetic construct
  source 1..34: mol_type = other DNA
cgcggatcccaccacgatcagcgactgggatcgc

SEQ ID NO: 116 - DNA, 29 bp, synthetic construct
  source 1..29: mol_type = other DNA
ccggaattccatgagcgttgaccagatca

SEQ ID NO: 117 - DNA, 40 bp, synthetic construct
  source 1..40: mol_type = other DNA
gccatccggcaagaccttatggtttatcctctttcgttac

SEQ ID NO: 118 - DNA, 40 bp, synthetic construct
  source 1..40: mol_type = other DNA
gtaacgaaagaggataaaccataaggtcttgccggatggc

SEQ ID NO: 119 - DNA, 34 bp, synthetic construct
  source 1..34: mol_type = other DNA
tgctctagaaaggcaatgtgctgcgcgaaggaag

SEQ ID NO: 120 - DNA, 29 bp, synthetic construct
  source 1..29: mol_type = other DNA
ccggaattcccggggccaacaaaacgggt

SEQ ID NO: 121 - DNA, 40 bp, synthetic construct
  source 1..40: mol_type = other DNA
caactttcagggtttgcatcgacattcctccagatttttg

SEQ ID NO: 122 - DNA, 40 bp, synthetic construct
  source 1..40: mol_type = other DNA
caaaaatctggaggaatgtcgatgcaaaccctgaaagttg

SEQ ID NO: 123 - DNA, 34 bp, synthetic construct
  source 1..34: mol_type = other DNA
cgcggatcccgatgaactcagggttcagaccaaa

SEQ ID NO: 124 - DNA, 29 bp, synthetic construct
  source 1..29: mol_type = other DNA
ccggaattcaccgtgttaagttcaagcgc

SEQ ID NO: 125 - DNA, 40 bp, synthetic construct
  source 1..40: mol_type = other DNA
ccgccacccggcatgtcggcaagactttctccagtgattt

SEQ ID NO: 126 - DNA, 40 bp, synthetic construct
  source 1..40: mol_type = other DNA
aaatcactggagaaagtcttgccgacatgccgggtggcgg

SEQ ID NO: 127 - DNA, 34 bp, synthetic construct
  source 1..34: mol_type = other DNA
cgcggatccggcgacggtcattatttcgcaggcg

SEQ ID NO: 128 - DNA, 29 bp, synthetic construct
  source 1..29: mol_type = other DNA
ccggaattcagtatatgaccgccaacggc

SEQ ID NO: 129 - DNA, 40 bp, synthetic construct
  source 1..40: mol_type = other DNA
gtgatttcagtcaattccaggtaacacctaccttcttaag

SEQ ID NO: 130 - DNA, 40 bp, synthetic construct
  source 1..40: mol_type = other DNA
cttaagaaggtaggtgttacctggaattgactgaaatcac

SEQ ID NO: 131 - DNA, 34 bp, synthetic construct
  source 1..34: mol_type = other DNA
tgctctagatgtatgccttctttgtggcaggcac

SEQ ID NO: 132 - DNA, 29 bp, synthetic construct
  source 1..29: mol_type = other DNA
ccggaattctcgtcagaaatcgagacatc

SEQ ID NO: 133 - DNA, 40 bp, synthetic construct
  source 1..40: mol_type = other DNA
gggtgagggaatcaggccacggtgaactcctcaatggaat

SEQ ID NO: 134 - DNA, 40 bp, synthetic construct
  source 1..40: mol_type = other DNA
attccattgaggagttcaccgtggcctgattccctcaccc

SEQ ID NO: 135 - DNA, 34 bp, synthetic construct
  source 1..34: mol_type = other DNA
tgctctagattcagtattctgattacgataaaat

SEQ ID NO: 136 - DNA, 29 bp, synthetic construct
  source 1..29: mol_type = other DNA
ccggaattcgaagacatattggcctccac

SEQ ID NO: 137 - DNA, 40 bp, synthetic construct
  source 1..40: mol_type = other DNA
tgttcacggtagttctcctgcatgctcgtcctcttcaact

SEQ ID NO: 138 - DNA, 40 bp, synthetic construct
  source 1..40: mol_type = other DNA
agttgaagaggacgagcatgcaggagaactaccgtgaaca

SEQ ID NO: 139 - DNA, 34 bp, synthetic construct
  source 1..34: mol_type = other DNA
tgctctagacaccgcccggcctgccgtgctcggc

SEQ ID NO: 140 - DNA, 29 bp, synthetic construct
  source 1..29: mol_type = other DNA
ccggaattcatcgcccgctacctctacag

SEQ ID NO: 141 - DNA, 40 bp, synthetic construct
  source 1..40: mol_type = other DNA
atgtcagagcttattagaatttctctgtccttatagtgag

SEQ ID NO: 142 - DNA, 40 bp, synthetic construct
  source 1..40: mol_type = other DNA
ctcactataaggacagagaaattctaataagctctgacat

SEQ ID NO: 143 - DNA, 34 bp, synthetic construct
  source 1..34: mol_type = other DNA
tgctctagattcgcccggctttttgtcggatttc

SEQ ID NO: 144 - DNA, 29 bp, synthetic construct
  source 1..29: mol_type = other DNA
ccggaattcaaacaatgagccgcgacgca

SEQ ID NO: 145 - DNA, 40 bp, synthetic construct
  source 1..40: mol_type = other DNA
tagcgcacccggcgtttttgaacatatctcccttagaggt

SEQ ID NO: 146 - DNA, 40 bp, synthetic construct
  source 1..40: mol_type = other DNA
acctctaagggagatatgttcaaaaacgccgggtgcgcta

SEQ ID NO: 147 - DNA, 34 bp, synthetic construct
  source 1..34: mol_type = other DNA
tgctctagacgattgccgacggtttccgcaacta

SEQ ID NO: 148 - DNA, 46 bp, synthetic construct
  source 1..46: mol_type = other DNA
aacagctatgacatgattacgaattcaccgtgttaagttcaagcgc

SEQ ID NO: 149 - DNA, 40 bp, synthetic construct
  source 1..40: mol_type = other DNA
gcggaacggtacttaggcataagactttctccagtgattt

SEQ ID NO: 150 - DNA, 40 bp, synthetic construct
  source 1..40: mol_type = other DNA
aaatcactggagaaagtcttatgcctaagtaccgttccgc

SEQ ID NO: 151 - DNA, 40 bp, synthetic construct
  source 1..40: mol_type = other DNA
ccgccacccggcatgtcggcttaaccccccagtttcgatt

SEQ ID NO: 152 - DNA, 40 bp, synthetic construct
  source 1..40: mol_type = other DNA
aatcgaaactggggggttaagccgacatgccgggtggcgg

SEQ ID NO: 153 - DNA, 51 bp, synthetic construct
  source 1..51: mol_type = other DNA
gcctgcaggtcgactctagaggatccggcgacggtcattatttcgcaggcg

SEQ ID NO: 154 - DNA, 46 bp, synthetic construct
  source 1..46: mol_type = other DNA
aacagctatgacatgattacgaattctcgtcagaaatcgagacatc

SEQ ID NO: 155 - DNA, 40 bp, synthetic construct
  source 1..40: mol_type = other DNA
atatatttaaaaagttccatggtgaactcctcaatggaat

SEQ ID NO: 156 - DNA, 40 bp, synthetic construct
  source 1..40: mol_type = other DNA
attccattgaggagttcaccatggaactttttaaatatat

SEQ ID NO: 157 - DNA, 40 bp, synthetic construct
  source 1..40: mol_type = other DNA
gggtgagggaatcaggccacttaacgtctgcttaatacac

SEQ ID NO: 158 - DNA, 40 bp, synthetic construct
  source 1..40: mol_type = other DNA
gtgtattaagcagacgttaagtggcctgattccctcaccc

SEQ ID NO: 159 - DNA, 51 bp, synthetic construct
  source 1..51: mol_type = other DNA
gcctgcaggtcgactctagaggatccttcagtattctgattacgataaaat

SEQ ID NO: 160 - DNA, 46 bp, synthetic construct
  source 1..46: mol_type = other DNA
aacagctatgacatgattacgaattcgaagacatattggcctccac

SEQ ID NO: 161 - DNA, 40 bp, synthetic construct
  source 1..40: mol_type = other DNA
gtattgaagtagttagccatcatgctcgtcctcttcaact

SEQ ID NO: 162 - DNA, 40 bp, synthetic construct
  source 1..40: mol_type = other DNA
agttgaagaggacgagcatgatggctaactacttcaatac

SEQ ID NO: 163 - DNA, 67 bp, synthetic construct; identical to SEQ ID NO: 71
  source 1..67: mol_type = other DNA

SEQ ID NO: 164 - DNA, 67 bp, synthetic construct; identical to SEQ ID NO: 72
  source 1..67: mol_type = other DNA

SEQ ID NO: 165 - DNA, 40 bp, synthetic construct
  source 1..40: mol_type = other DNA
tgttcacggtagttctcctgttaacccgcaacagcaatac

SEQ ID NO: 166 - DNA, 40 bp, synthetic construct
  source 1..40: mol_type = other DNA
gtattgctgttgcgggttaacaggagaactaccgtgaaca

SEQ ID NO: 167 - DNA, 51 bp, synthetic construct
  source 1..51: mol_type = other DNA
gcctgcaggtcgactctagaggatcccaccgcccggcctgccgtgctcggc

SEQ ID NO: 168 - DNA, 29 bp, synthetic construct
  source 1..29: mol_type = other DNA
agcgaattcatggctaactacttcaatac

SEQ ID NO: 169 - DNA, 40 bp, synthetic construct
  source 1..40: mol_type = other DNA
atctcttgcgttttttgcacttaacccgcaacagcaatac

SEQ ID NO: 170 - DNA, 40 bp, synthetic construct
  source 1..40: mol_type = other DNA
gtattgctgttgcgggttaagtgcaaaaaacgcaagagat

SEQ ID NO: 171 - DNA, 39 bp, synthetic construct
  source 1..39: mol_type = other DNA
cttttgttgcttttgtcaattagaaaagattcaccagtc

SEQ ID NO: 172 - DNA, 40 bp, synthetic construct
  source 1..40: mol_type = other DNA
gactggtgaatcttttctaattgacaaaagcaacaaaaga

SEQ ID NO: 173 - DNA, 29 bp, synthetic construct
  source 1..29: mol_type = other DNA
agcggatccctagagagctttcgttttca

SEQ ID NO: 174 - DNA, 29 bp, synthetic construct
  source 1..29: mol_type = other DNA
ccggggcccgtacctgtcgcgggtgtgac

SEQ ID NO: 175 - DNA, 40 bp, synthetic construct
  source 1..40: mol_type = other DNA
cgatctcccttggcatcataatcacgtcctcctttgtttt

SEQ ID NO: 176 - DNA, 40 bp, synthetic construct
  source 1..40: mol_type = other DNA
aaaacaaaggaggacgtgattatgatgccaagggagatcg

SEQ ID NO: 177 - DNA, 34 bp, synthetic construct
  source 1..34: mol_type = other DNA
cgcggatccgggggcggtatatgtccaggtaaag

SEQ ID NO: 178 - DNA, 29 bp, synthetic construct
  source 1..29: mol_type = other DNA
ccggggccccgacgatgctgtaaagcgta

SEQ ID NO: 179 - DNA, 40 bp, synthetic construct
  source 1..40: mol_type = other DNA
cacctttttcaggaagcctatatataccctccttgaaagt

SEQ ID NO: 180 - DNA, 40 bp, synthetic construct
  source 1..40: mol_type = other DNA
actttcaaggagggtatatataggcttcctgaaaaaggtg

SEQ ID NO: 181 - DNA, 34 bp, synthetic construct
  source 1..34: mol_type = other DNA
tgcggatccaagaaaagcgattatctttatacat

SEQ ID NO: 182 - DNA, 29 bp, synthetic construct
  source 1..29: mol_type = other DNA
ccggggcccaaagtaaaaaatgattccgt

SEQ ID NO: 183 - DNA, 40 bp, synthetic construct
  source 1..40: mol_type = other DNA
gcatgcagccggtttgttgatttcttatcccttccttctc

SEQ ID NO: 184 - DNA, 40 bp, synthetic construct
  source 1..40: mol_type = other DNA
gagaaggaagggataagaaatcaacaaaccggctgcatgc

SEQ ID NO: 185 - DNA, 34 bp, synthetic construct
  source 1..34: mol_type = other DNA
cgcggatccaagaaaagcgattatctttatacat

SEQ ID NO: 186 - DNA, 29 bp, synthetic construct
  source 1..29: mol_type = other DNA
ccggggccctataaaaaagatgacaacaa

SEQ ID NO: 187 - DNA, 40 bp, synthetic construct
  source 1..40: mol_type = other DNA
agtatcttcatggtgttcaggactcatcattcctttgccg

SEQ ID NO: 188 - DNA, 40 bp, synthetic construct
  source 1..40: mol_type = other DNA
cggcaaaggaatgatgagtcctgaacaccatgaagatact

SEQ ID NO: 189 - DNA, 34 bp, synthetic construct
  source 1..34: mol_type = other DNA
cgcggatccgtttaaaaccaagctcgacaagaag

SEQ ID NO: 190 - DNA, 29 bp, synthetic construct
  source 1..29: mol_type = other DNA
ccggggccctgacttctcccattgcagca

SEQ ID NO: 191 - DNA, 40 bp, synthetic construct
  source 1..40: mol_type = other DNA
cgcgctccgcttattgctcgttaaatccccctctttttca

SEQ ID NO: 192 - DNA, 40 bp, synthetic construct
  source 1..40: mol_type = other DNA
tgaaaaagagggggatttaacgagcaataagcggagcgcg

SEQ ID NO: 193 - DNA, 34 bp, synthetic construct
  source 1..34: mol_type = other DNA
tgcggatccggcattcctgtcaggttgatatgtt

SEQ ID NO: 194 - DNA, 29 bp, synthetic construct
  source 1..29: mol_type = other DNA
ccggggccctcgctgaaaaactaaaagaa

SEQ ID NO: 195 - DNA, 40 bp, synthetic construct
  source 1..40: mol_type = other DNA
cggaatgacggcttttttggtgtaaaccctccagtgaatg

SEQ ID NO: 196 - DNA, 40 bp, synthetic construct
  source 1..40: mol_type = other DNA
cattcactggagggtttacaccaaaaaagccgtcattccg

SEQ ID NO: 197 - DNA, 34 bp, synthetic construct
  source 1..34: mol_type = other DNA
tgcggatcctgcgaatggttgtacttcttttccg

SEQ ID NO: 198 - DNA, 29 bp, synthetic construct
  source 1..29: mol_type = other DNA
ccggggccctggggtggctttgccgtggg

SEQ ID NO: 199 - DNA, 40 bp, synthetic construct
  source 1..40: mol_type = other DNA
ttcaaagagggctttttcattttcctcttttcactccctt

SEQ ID NO: 200 - DNA, 40 bp, synthetic construct
  source 1..40: mol_type = other DNA
aagggagtgaaaagaggaaaatgaaaaagccctctttgaa

SEQ ID NO: 201 - DNA, 34 bp, synthetic construct
  source 1..34: mol_type = other DNA
tgcggatccttgaagcgatcagaagctcagggaa

SEQ ID NO: 202 - DNA, 29 bp, synthetic construct
  source 1..29: mol_type = other DNA
ccggggcccaaagcgcatgttttaaaacc

SEQ ID NO: 203 - DNA, 40 bp, synthetic construct
  source 1..40: mol_type = other DNA
tatagaatataattttaaaaataaacatcttctttctata

SEQ ID NO: 204 - DNA, 40 bp, synthetic construct
  source 1..40: mol_type = other DNA
tatagaaagaagatgtttatttttaaaattatattctata

SEQ ID NO: 205 - DNA, 34 bp, synthetic construct; identical to SEQ ID NO: 201
  source 1..34: mol_type = other DNA

SEQ ID NO: 206 - DNA, 30 bp, synthetic construct
  source 1..30: mol_type = other DNA
atttagatctaacaagccgcgtcattcaag

SEQ ID NO: 207 - DNA, 38 bp, synthetic construct
  source 1..38: mol_type = other DNA
acttggcgccattcttcttcgacacatcgcaaatgata

SEQ ID NO: 208 - DNA, 38 bp, synthetic construct
  source 1..38: mol_type = other DNA
tatcatttgcgatgtgtcgaagaagaatggcgccaagt

SEQ ID NO: 209 - DNA, 20 bp, synthetic construct
  source 1..20: mol_type = other DNA
taccgtggatccgctttaag

SEQ ID NO: 210 - DNA, 29 bp, synthetic construct
  source 1..29: mol_type = other DNA
ccggggccctataaaaaagatgacaacaa

SEQ ID NO: 211 - DNA, 40 bp, synthetic construct
  source 1..40: mol_type = other DNA
gcggaacggtacttaggcatgactcatcattcctttgccg

SEQ ID NO: 212 - DNA, 40 bp, synthetic construct
  source 1..40: mol_type = other DNA
cggcaaaggaatgatgagtcatgcctaagtaccgttccgc

SEQ ID NO: 213 - DNA, 40 bp, synthetic construct
  source 1..40: mol_type = other DNA
agtatcttcatggtgttcagttaaccccccagtttcgatt

SEQ ID NO: 214 - DNA, 40 bp, synthetic construct
  source 1..40: mol_type = other DNA
aatcgaaactggggggttaactgaacaccatgaagatact

SEQ ID NO: 215 - DNA, 34 bp, synthetic construct; identical to SEQ ID NO: 189
  source 1..34: mol_type = other DNA

SEQ ID NO: 216 - DNA, 29 bp, synthetic construct
  source 1..29: mol_type = other DNA
ccggggccctcgctgaaaaactaaaagaa

SEQ ID NO: 217 - DNA, 40 bp, synthetic construct
  source 1..40: mol_type = other DNA
atatatttaaaaagttccattgtaaaccctccagtgaatg

SEQ ID NO: 218 - DNA, 40 bp, synthetic construct
  source 1..40: mol_type = other DNA
cattcactggagggtttacaatggaactttttaaatatat

SEQ ID NO: 219 - DNA, 40 bp, synthetic construct
  source 1..40: mol_type = other DNA
cggaatgacggcttttttggttaacgtctgcttaatacac

SEQ ID NO: 220 - DNA, 40 bp, synthetic construct
  source 1..40: mol_type = other DNA
gtgtattaagcagacgttaaccaaaaaagccgtcattccg

SEQ ID NO: 221 - DNA, 34 bp, synthetic construct; identical to SEQ ID NO: 197
  source 1..34: mol_type = other DNA

SEQ ID NO: 222 - DNA, 29 bp, synthetic construct
  misc_feature 1: note = 1
  source 1..29: mol_type = other DNA
ccggggccctggggtggctttgccgtggg

SEQ ID NO: 223 - DNA, 40 bp, synthetic construct
  source 1..40: mol_type = other DNA
gtattgaagtagttagccattttcctcttttcactccctt

SEQ ID NO: 224 - DNA, 40 bp, synthetic construct
  source 1..40: mol_type = other DNA
aagggagtgaaaagaggaaaatggctaactacttcaatac

SEQ ID NO: 225 - DNA, 67 bp, synthetic construct; identical to SEQ ID NO: 71
  source 1..67: mol_type = other DNA

SEQ ID NO: 226 - DNA, 67 bp, synthetic construct; identical to SEQ ID NO: 72
  source 1..67: mol_type = other DNA

SEQ ID NO: 227 - DNA, 40 bp, synthetic construct
  source 1..40: mol_type = other DNA
ttcaaagagggctttttcatttaacccgcaacagcaatac

SEQ ID NO: 228 - DNA, 40 bp, synthetic construct
  source 1..40: mol_type = other DNA
gtattgctgttgcgggttaaatgaaaaagccctctttgaa

SEQ ID NO: 229 - DNA, 34 bp, synthetic construct; identical to SEQ ID NO: 201
  source 1..34: mol_type = other DNA

SEQ ID NO: 230 - DNA, 29 bp, synthetic construct
  source 1..29: mol_type = other DNA
ccggggccctgacttctcccattgcagca

SEQ ID NO: 231 - DNA, 40 bp, synthetic construct
  source 1..40: mol_type = other DNA
atctcttgcgttttttgcacttaaatccccctctttttca

SEQ ID NO: 232 - DNA, 40 bp, synthetic construct
  source 1..40: mol_type = other DNA
tgaaaaagagggggatttaagtgcaaaaaacgcaagagat

SEQ ID NO: 233 - DNA, 40 bp, synthetic construct
  source 1..40: mol_type = other DNA
cgcgctccgcttattgctcgttagaaaagattcaccagtc

SEQ ID NO: 234 - DNA, 40 bp, synthetic construct
  source 1..40: mol_type = other DNA
gactggtgaatcttttctaacgagcaataagcggagcgcg

SEQ ID NO: 235 - DNA, 34 bp, synthetic construct; identical to SEQ ID NO: 193
  source 1..34: mol_type = other DNA

SEQ ID NO: 236 - DNA, 29 bp, synthetic construct
  source 1..29: mol_type = other DNA
ccggggcccaaagcgcatgttttaaaacc

SEQ ID NO: 237 - DNA, 40 bp, synthetic construct
  source 1..40: mol_type = other DNA
tcttttgttgcttttgtcaaataaacatcttctttctata

SEQ ID NO: 238 - DNA, 40 bp, synthetic construct
  source 1..40: mol_type = other DNA
tatagaaagaagatgtttatttgacaaaagcaacaaaaga

SEQ ID NO: 239 - DNA, 40 bp, synthetic construct
  source 1..40: mol_type = other DNA
tatagaatataattttaaaactagagagctttcgttttca

SEQ ID NO: 240 - DNA, 40 bp, synthetic construct
  source 1..40: mol_type = other DNA
tgaaaacgaaagctctctagttttaaaattatattctata

SEQ ID NO: 241 - DNA, 34 bp, synthetic construct; identical to SEQ ID NO: 201
  source 1..34: mol_type = other DNA

SEQ ID NO: 242 - DNA, 30 bp, synthetic construct; identical to SEQ ID NO: 206
  source 1..30: mol_type = other DNA

SEQ ID NO: 243 - DNA, 40 bp, synthetic construct
  source 1..40: mol_type = other DNA
gtattgaagtagttagccatggtaattcccccttcactat

SEQ ID NO: 244 - DNA, 40 bp, synthetic construct
  source 1..40: mol_type = other DNA
atagtgaagggggaattaccatggctaactacttcaatac

SEQ ID NO: 245 - DNA, 40 bp, synthetic construct
  source 1..40: mol_type = other DNA
cggaaacggcttttcgtctattaacccgcaacagcaatac

SEQ ID NO: 246 - DNA, 40 bp, synthetic construct
  source 1..40: mol_type = other DNA
gtattgctgttgcgggttaatagacgaaaagccgtttccg

SEQ ID NO: 247 - DNA, 20 bp, synthetic construct
  source 1..20: mol_type = other DNA
taccgtggatccgctttaag

(19) United States
(12) Patent Application Publication
(10) Pub. No.: US 2026/0028652 A1
(43) Pub. Date: Jan. 29, 2026

(54) METHOD FOR CONSTRUCTING L-VALINE PRODUCING STRAIN, L-VALINE PRODUCING STRAIN AND USE THEREOF

(71) Applicant: SHANDONG UNIVERSITY, Qingdao (CN)

(72) Inventors: Chao GAO, Qingdao (CN); Menghao CAO, Qingdao (CN); Cuiqing MA, Qingdao (CN); Ping XU, Shanghai (CN)

(21) Appl. No.: 19/138,716

(22) PCT Filed: Mar. 6, 2023
(86) PCT No.: PCT/CN2023/079828

§ 371 Date: Jun. 13, 2025

(30) Foreign Application Priority Data
Dec. 16, 2022 (CN) 202211622249.7

(51) Int. Cl. C12P 13/08; C12N 9/02; C12N 9/10; C12N 9/88; C12N 15/52; C12N 15/74

(52) U.S. Cl. CPC C12P 13/08; C12N 9/001; C12N 9/1022; C12N 9/88; C12N 15/52; C12N 15/74; C12Y 103/08004; C12Y 202/01006; C12Y 402/01009; C12N 2800/101

(57) ABSTRACT

The present invention provides a method for constructing an L-valine production strain, the L-valine production strain, and use thereof. According to the method for constructing the L-valine-producing strain, a 2,3-butanediol- or acetoin-producing strain is used as a starting strain, and genetic engineering modification is performed on the strain to improve the L-valine yield thereof. The present invention provides a new thought and way for efficient production of L-valine, and obtains a new production strain for efficiently producing L-valine. The L-valine-producing strain obtained in the present invention requires a simple culture medium and has low fermentation substrate and culture costs; meanwhile, the strain has a high L-valine yield and has a single product component easy to separate.

### CROSS-REFERENCE TO RELATED APPLICATIONS

This application is a 371 of international application of PCT application serial no. PCT/CN2023/079828, filed on Mar. 6, 2023, which claims the priority benefit of China application no. 202211622249.7, filed on Dec. 16, 2022. The entirety of each of the above-mentioned patent applications is hereby incorporated by reference herein and made a part of this specification.

### REFERENCE TO SEQUENCE LISTING

The instant application contains a Sequence Listing in XML format as a file named “PCTW2502US-Sequence-Listing.xml”, created on Jun. 11, 2025, of 273,961 bytes in size, and which is hereby incorporated by reference in its entirety.

### TECHNICAL FIELD

The present invention belongs to the technical field of bioengineering, and in particular relates to a method for constructing an L-valine producing strain, an L-valine producing strain, and use thereof.

### BACKGROUND

L-Valine is a branched-chain amino acid (BCAA) widely applied in food, pharmaceutical, cosmetic and the like industries (Wu G et al., Amino Acids, 2009, 37:1-17). Moreover, the L-valine can improve the lactation function of a domestic animal in a lactation period and enhance the immunity of the animal, and is also applied in the feed industry (Park Y et al., Molecules, 2016, 21:1272; Park J H et al., Proc Natl Acad Sci USA, 2007, 104:7797-7802). The L-valine can be directly acquired from a hydrolyzate solution of a meat processing waste treated with subcritical water using ion exchange technology (Zhu G et al., J Anal Appl Pyrol, 2010, 88:187-191). This process is high in cost, low in efficiency, complex in reactions and difficult to control. Therefore, the efficient production of the L-valine achieved through microbial fermentation technology has attracted the attention of researchers.

Corynebacterium glutamicum and Escherichia coli are the main strains currently used for fermentation production of L-valine (Hasegawa S et al., Appl Environ Microbiol, 2012, 78:865-875; Mustafi N et al., PLoS One, 2014, 9: e85731; Park J H et al., Biotechnol Bioeng, 2011, 108: 1140-1147; Hao Y et al., Bioresour Technol, 2022, 359: 127461). Currently, the L-valine produced by fermentation of C. glutamicum has a maximum production of 86.5 g/L (with a fermentation volume of 300 milliliters, and a fermentation time of 55 h), a yield of 0.234 g/g, and production efficiency of 1.57 g/L/h (Buchholz J et al., Appl Environ Microbiol, 2013, 79: 5566-75). Recently, Hao et al. has screened out a strain of E. coli W3110 that naturally accumulates the L-valine through ARTP mutagenesis technology, and has further carried out system metabolic engineering on the strain. Under low dissolved oxygen conditions, the production of the L-valine can reach 92.0 g/L (with a fermentation volume of 2 liters, and a fermentation time of 55 hours), but the yield of the L-valine is only 0.340 g/g, and the production efficiency of it is 1.92 g/L/h (Hao Y et al., Bioresour Technol, 2022, 359: 127461). Moreover, Jiang Yu et al. has disclosed a method for constructing an L-valine producing strain using E. coli ATCC8739 as a chassis strain, with production of the L-valine reaching 94.8 g/L (with a fermentation volume of 25 liters and a fermentation time of 50 hours), a yield of the L-valine being 0.578 g/g, and production efficiency of it being 1.90 g/L/h (CN114958888A). However, the efficiency of L-valine synthesis in these existing technologies is still limited, and the production and substrate conversion rate of the L-valine are low, making it difficult to meet the requirements of industrial production.

### SUMMARY

In view of the deficiencies in the prior art, an objective of the present invention is to provide a method for constructing an L-valine producing strain, an L-valine producing strain, and use thereof.

To achieve the aforementioned objective, solutions of the present invention are:
(1) a method for constructing an L-valine producing strain, including genetically engineering a 2,3-butanediol or acetoin producing strain as a starting strain to increase L-valine production thereof.
(2) The method according to (1), where the strain is subjected to the following engineering: 1) increasing the synthesis of α-acetolactate; and 2) introducing an exogenous L-valine biosynthetic pathway.
(3) The method according to (1), where the starting strain is selected from microorganisms of the genera Klebsiella, Enterobacter, Bacillus, Corynebacterium and Vibrio.
(4) The method according to (3), where the starting strain is selected from Klebsiella oxytoca, Enterobacter cloacae, Escherichia coli, Vibrio natriegens, Corynebacterium glutamicum and Bacillus licheniformis.
(5) The method according to (2), where the increasing the synthesis of α-acetolactate includes: i) inhibiting the synthesis of acetoin and/or 2,3-butanediol; and/or ii) inhibiting the synthesis of acetic acid, formic acid, ethanol, succinic acid and/or lactic acid.
(6) The method according to (5), where the inhibiting the synthesis of acetoin and/or 2,3-butanediol includes knocking out or knocking down one or more of the following coding genes in the starting strain: an α-acetolactate decarboxylase coding gene budA, a 2,3-butanediol dehydrogenase coding gene budC and a glycerol dehydrogenase coding gene gldA.
(7) The method according to (5), where the inhibiting the synthesis of acetic acid, formic acid, ethanol, succinic acid and/or lactic acid includes knocking out or knocking down one or more of the following coding genes in the starting strain: a pyruvate oxidase coding gene pox, a phosphotransacetylase coding gene pta, a fumarate reductase subunit A coding gene frdA, a lactate dehydrogenase coding gene ldh, a pyruvate formate lyase coding gene pflB and an ethanol dehydrogenase coding gene adhE.
(8) The method according to (2), where the introducing an exogenous L-valine biosynthetic pathway includes introducing coding sequences of one or more of the following genes into the starting strain: a dihydroxyacid dehydratase gene, an L-leucine dehydrogenase gene and an acetohydroxyacid isomeroreductase gene.
(9) The method according to (8), where the introducing an exogenous L-valine biosynthetic pathway includes introducing coding sequence of one or more of the following genes into the starting strain: a dihydroxyacid dehydratase gene puDHT, a dihydroxyacid dehydratase gene dhaD, a dihydroxyacid dehydratase gene ilvD, an L-leucine dehydrogenase gene bed, and an acetohydroxyacid isomeroreductase cofactor preference mutant protein gene ilvC^{M}.
(10) The method according to (2), where the engineering further includes optimizing L-valine synthetic flux and/or enhancing L-valine efflux in the starting strain.
(11) The method according to (10), where the optimizing L-valine synthetic flux and/or enhancing L-valine efflux in the starting strain includes introducing coding sequences of one or more of the following genes into the starting strain: the acetohydroxyacid isomeroreductase cofactor preference mutant protein gene ilvC^{M}, a branched-chain amino acid transporter gene brnFE, a branched-chain amino acid transporter gene ygaZH, an α-acetolactate synthase gene alsS, an α-acetolactate synthase gene budB, an acetohydroxyacid isomeroreductase gene ilvC, a dihydroxyacid dehydratase gene dhaD, and a dihydroxyacid dehydratase gene ilvD.
(12) The method according to any one of (8), (9) and (11), where the introducing coding sequences into the starting strain includes integrating the coding sequences into a genome of the starting strain or expressing the coding sequence in the starting strain in a plasmid form; preferably, the introducing includes introducing a single copy or multiple copies of the coding sequences of the genes; and preferably, the coding sequences of the genes are introduced in the form of respective single gene expression fragments or in the form of tandem expression fragments of the coding sequences of the genes.
(13) The method according to (9), where the dihydroxyacid dehydratase gene puDHT is derived from Paralcaligenes ureilyticus, the dihydroxyacid dehydratase gene dhaD is derived from Sulfolobus solfataricus, the dihydroxyacid dehydratase gene ilvD is derived from Escherichia coli, the L-leucine dehydrogenase gene bcd is derived from Bacillus subtilis, and the acetohydroxyacid isomeroreductase cofactor preference mutant protein gene ilvC^{M} is derived from Escherichia coli.
(14) The method according to (11), where the acetohydroxyacid isomeroreductase cofactor preference mutant protein gene ilvC^{M} is derived from Escherichia coli, the branched-chain amino acid transporter gene brnFE is derived from Corynebacterium glutamicum, the branched-chain amino acid transporter gene ygaZH is derived from Escherichia coli, the acetohydroxyacid isomeroreductase gene ilvC is derived from Escherichia coli, the dihydroxyacid dehydratase gene dhaD is derived from Sulfolobus solfataricus, the dihydroxyacid dehydratase gene ilvD is derived from Escherichia coli, the α-acetolactate synthase gene alsS is derived from Bacillus subtilis, and the α-acetolactate synthase gene budB is derived from Klebsiella pneumoniae.
(15) An L-valine producing strain, which is constructed by the method according to any one of (1)-(14).
(16) The strain according to (15), where the strain is Klebsiella oxytoca with a deposit number of CCTCC M 20221743.
(17) Use of the strain according to (15) or (16) in production of L-valine.
(18) The use according to (17), where at a fermentation volume of 5 liters and a fermentation time of 30-56 hours, the production of L-valine is 45.1-122.0 g/L, the production intensity is 1.41-2.18 g/L/h, and the yield is 0.246-0.587 g/g.
(19) A method for producing L-valine, including the following steps:
1) providing the L-valine producing strain according to (15) or (16);
2) culturing the strain at 30-50° C. for 10-11 hours to provide a seed; and
3) fermenting the seed with glucose as a substrate at 30-50° C., a pH value of 6.0-7.0 and a ventilation volume of 0.5-1.6 vvm to obtain L-valine,
(20) The method according to (19), where in the step 3), the inoculation amount of the seed is an OD₆₂₀ₙₘ value reaching 0.2-0.8; preferably, the concentration of glucose is 40-60 g/L; and preferably, the fermentation culture is a stirred culture with a stirring rate of 300-550 rpm.

By employing the aforementioned solutions, the present invention has the following beneficial effects.

The present invention proposes to achieve efficient production of L-valine by modifying the metabolic pathway of the 2,3-butanediol or acetoin producing strain as the starting strain. On the basis of this construction strategy, in the present invention genes related to the synthesis of the acetic acid, formic acid, ethanol, succinic acid and lactic acid are knocked out, and at the same time endogenous or exogenous α-acetolactate synthase is overexpressed, thereby improving the synthesis efficiency of α-acetolactate; meanwhile, α-acetolactate is blocked from entering the 2,3-butanediol synthesis pathway, and an exogenous L-valine biosynthesis pathway is introduced, so that he intracellular α-acetolactate metabolic flow is redirected from 2,3-butanediol synthesis to the exogenous L-valine synthesis pathway, thereby achieving the efficient synthesis of L-valine. The present invention can also achieve more efficient production of L-valine by further optimizing L-valine synthesis flux, enhancing L-valine efflux and the like strategies. Therefore, the present invention provides a new idea and approach for the efficient production of L-valine, and obtains a new production strain for the efficient production of L-valine.

The optimal engineered strain of Klebsiella oxytoca of the present invention is utilized to produce L-valine with glucose as a substrate under the fermentation conditions given by the present invention (a fermentation volume of 5 liters, and a fermentation time of 56 hours), where the yield can reach 122.0 g/L, and a yield is 0.587 g/g. By utilizing the optimal engineered strain of Enterobacter cloacae of the present invention with glucose as a substrate, under the fermentation conditions given in the present invention (a fermentation volume of 5 liters, and a fermentation time of 44 hours), L-valine is produced at a concentration reaching 94.3 g/L, production intensity reaching 2.14 g/L/h, and an L-valine yield reaching 0.499 g/g. By utilizing the optimal engineered strain of Bacillus licheniformis of the present invention with glucose as a substrate, under the fermentation conditions given in the present invention (a fermentation volume of 5 liters, and a fermentation time of 32 hours), L-valine is produced at a concentration reaching 45.1 g/L, production intensity reaching 1.41 g/L/h, and an L-valine yield reaching 0.246 g/g.

For the L-valine producing strain obtained by the present invention, the required medium is simple, the fermentation substrate and the culture are low in cost, and meanwhile the strain has a high yield of L-valine, and the product ingredient is single and easy to separate.

### Biomaterial Deposit Information

The Klebsiella oxytoca VKO-9 strain provided by the present invention has been deposited in the China Center for Type Culture Collection (CCTCC) on Nov. 9, 2022, at the deposit address: Wuhan University, Wuhan, China, Postal Code: 430072, and the deposit number is: CCTCC M 20221743.

### BRIEF DESCRIPTION OF DRAWINGS

FIG. 1 shows a schematic diagram of an L-valine synthetic pathway and metabolic engineering strategy in Klebsiella oxytoca VKO-9 constructed according to an embodiment of the present invention.

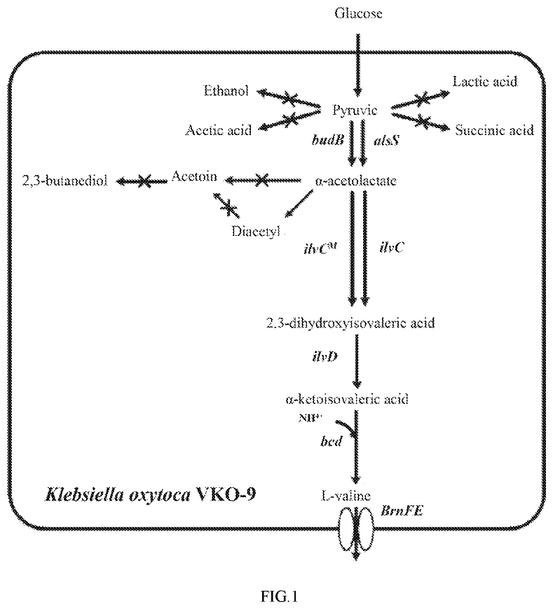

### DETAILED DESCRIPTION

The present invention is described in detail hereafter in conjunction with specific embodiments, so as to more clearly embody the aforementioned features and advantages of the present invention. The examples described hereafter are merely preferred embodiments of the present invention and are not intended to limit the present invention in any form. Therefore, the present invention is not limited to the specific examples disclosed hereafter in the specification.

Microorganisms such as Klebsiella oxytoca can efficiently synthesize 2,3-butanediol or acetoin by metabolizing carbohydrates such as glucose, etc. (Jantama K., Metab Eng., 2015, 30: 16-26). The intermediate metabolites in the 2,3-butanediol and acetoin biosynthesis pathway include α-acetolactate. In the present invention, an efficient L-valine producing strain is constructed by utilizing the characteristics of α-acetolactate that can be used as a precursor for the synthesis of L-valine as well as the strong α-acetolactate synthesis ability of 2,3-butanediol or acetoin producing strains, and adopting a metabolic flow redirection strategy. The present invention proposes that the synthesis efficiency of the α-acetolactate should be improved, while blocking the α-acetolactate from entering the 2,3-butanediol synthesis pathway, introducing an exogenous L-valine biosynthesis pathway, and redirecting the intracellular α-acetolactate metabolic flow from 2,3-butanediol synthesis to the exogenous L-valine synthesis pathway, thereby achieving efficient synthesis of L-valine.

Based on the aforementioned inventive concept, the present invention provides a method for constructing an L-valine producing strain, including genetically engineering a 2,3-butanediol or acetoin producing strain as a starting strain to increase L-valine production thereof.

Further, the present invention proposes to carry out the following engineering to the strain, which can effectively improve the production of L-valine: 1) increasing the synthesis of α-acetolactate; and 2) introducing an exogenous L-valine biosynthetic pathway.

The term “starting strain” used herein refers to a strain that has not undergone the genetic engineering described in the present invention, which may be a wild-type strain or a recombinant strain that has undergone some or certain known modification(s) previously.

The term “exogenous” as used herein when referring to genes, coding sequences, proteins, enzymes, etc., refers to a substance that does not belong to the starting strain in a natural state. For example, an exogenous gene refers to a gene introduced from outside into a starting strain. The exogenous gene may be a gene already present in the genome of the strain or a gene not present in the genome of the strain. For example, as desired, a gene already present in the genome of the starting strain can be introduced from outside into the genome of the starting strain to overexpress the gene.

Preferably, the starting strain is selected from microorganisms of the genera Klebsiella, Enterobacter, Bacillus and Vibrio. More preferably, the starting strain is selected from Klebsiella oxytoca, Enterobacter cloacae, Escherichia coli, Vibrio natriegens, Corynebacterium glutamicum and Bacillus licheniformis.

In some specific preferred embodiments, the starting strain is selected from Klebsiella oxytoca PDL-0, Enterobacter cloacae SDM, Escherichia coli BL21/pET-RABC, Bacillus licheniformis 10-1-A, Corynebacterium glutamicum ATCC13032/pEKEx2-als, aldB, P_{tuf}-butA and Vibrio natriegens ATCC14048/pET-RABC.

The present invention can increase the synthesis of α-acetolactate by inhibiting the synthesis of acetoin and/or 2,3-butanediol and/or inhibiting the synthesis of by-products such as acetic acid, formic acid, ethanol, succinic acid, lactic acid, etc. A specific preferred embodiment for inhibiting the synthesis of acetoin and/or 2,3-butanediol includes knocking out or knocking down one or more of the following coding genes in the starting strain: an α-acetolactate decarboxylase coding gene budA, a 2,3-butanediol dehydrogenase coding gene budC and a glycerol dehydrogenase coding gene gldA. A specific preferred embodiment for inhibiting the synthesis of acetic acid, formic acid, ethanol, succinic acid and/or lactic acid includes knocking out or knocking down one or more of the following coding genes in the starting strain: a pyruvate oxidase coding gene pox, a phosphotransacetylase coding gene pta, a fumarate reductase subunit A coding gene frdA, a lactate dehydrogenase coding gene ldh, a pyruvate formate lyase coding gene pflB and an ethanol dehydrogenase coding gene adhE.

Unless otherwise specified, the “lactate dehydrogenase coding gene ldh” described herein refers to a D-lactate dehydrogenase coding gene ldhD and/or an L-lactate dehydrogenase coding gene ldhL.

The term “inhibit” as used herein refers to that the function of the object of inhibition is completely lost or reduced compared with that before the inhibition is implemented.

The term “knockout” or “knockdown” as used herein refers to the loss or weakening of the function of a selected gene by genetic engineering means, including those commonly used in the art, e.g. inserting, substituting or deleting one or more nucleic acid fragments in the selected gene.

Preferably, the introducing an exogenous L-valine biosynthetic pathway includes introducing coding sequences of one or more of the following genes into the starting strain: a dihydroxyacid dehydratase gene, an L-leucine dehydrogenase gene and an acetohydroxyacid isomeroreductase gene. In some specific preferred embodiments, the coding sequences of one or more of the following genes are introduced into the starting strain: a dihydroxyacid dehydratase gene puDHT, a dihydroxyacid dehydratase gene dhaD, a dihydroxyacid dehydratase gene ilvD, an L-leucine dehydrogenase gene bcd, and an ilvD^{M}.

In some specific preferred embodiments, the dihydroxyacid dehydratase gene puDHT is derived from Paralcaligenes ureilyticus, the dihydroxyacid dehydratase gene dhaD is derived from Sulfolobus solfataricus, the dihydroxyacid dehydratase gene ilvD is derived from Escherichia coli, the L-leucine dehydrogenase gene bcd is derived from Bacillus subtilis, and the acetohydroxyacid isomeroreductase cofactor preference mutant protein gene ilvC^{M} is derived from Escherichia coli.

Preferably, the dihydroxyacid dehydratase gene puDHT, the dihydroxyacid dehydratase gene ilvD or the dihydroxyacid dehydratase gene dhaD is inserted into a gene site of the lactate dehydrogenase gene ldh of the starting strain. Preferably, the L-leucine dehydrogenase gene bcd is inserted into the gene site of the ethanol dehydrogenase gene adhE of the starting strain. Preferably, the acetohydroxyacid isomeroreductase cofactor preference mutant protein gene ilvC^{M} is inserted into a gene site of the α-acetolactate decarboxylase gene budA of the starting strain. In this way, the synthesis of 2,3-butanediol/acetoin can be inhibited or blocked, the synthesis of α-acetolactate is increased, and redirection of the α-acetolactate metabolic flux into the L-valine synthesis pathway is realized. Moreover, in the present invention an exogenous gene is inserted into the site of the knocked-out gene, which can satisfy the expression of the target gene as much as possible without affecting other genes.

The method of the present invention further includes optimizing L-valine synthesis flux and/or enhancing L-valine efflux in the starting strain, thereby obtaining an engineered strain that can more efficiently produce L-valine.

Preferably, the optimizing L-valine synthetic flux and/or enhancing L-valine efflux in the starting strain includes introducing coding sequences of one or more of the following genes into the starting strain: the acetohydroxyacid isomeroreductase cofactor preference mutant protein gene ilvC^{M}, a branched-chain amino acid transporter gene brnFE, a branched-chain amino acid transporter gene ygaZH, an acetohydroxyacid isomeroreductase gene ilvC, a dihydroxyacid dehydratase gene dhaD, a dihydroxyacid dehydratase gene ilvD, an α-acetolactate synthase gene alsS, an α-acetolactate synthase gene budB.

In the case of Klebsiella oxytoca, preferably, the aforementioned dihydroxyacid dehydratase gene puDHT introduced into the starting strain is replaced by the dihydroxyacid dehydratase gene dhaD) and/or the dihydroxyacid dehydratase gene ilvD, thereby optimizing the source of the dihydroxyacid dehydratase and further improving the synthesis of L-valine.

In the step of optimizing the synthetic flux and enhancing the efflux, preferably, the acetohydroxyacid isomeroreductase cofactor preference mutant protein gene ilvC^{M} is derived from Escherichia coli, the branched-chain amino acid transporter gene brnFE is derived from Corynebacterium glutamicum, the branched-chain amino acid transporter gene ygaZH is derived from Escherichia coli, the acetohydroxyacid isomeroreductase gene ilvC is derived from Escherichia coli, the dihydroxyacid dehydratase gene dhaD is derived from Sulfolobus solfataricus, the dihydroxyacid dehydratase gene ilvD is derived from Escherichia coli, the α-acetolactate synthase gene alsS is derived from Bacillus subtilis, and the α-acetolactate synthase gene budB is derived from Klebsiella pneumoniae.

In the step of optimizing the synthetic flux and enhancing the efflux, preferably, the acetohydroxyacid isomeroreductase cofactor preference mutant protein gene ilvC^{M} and/or the acetohydroxyacid isomeroreductase gene ilvC are inserted into the site of glycerol dehydrogenase gene gldA of the starting strain. In this way, the copy number of the acetohydroxyacid isomeroreductase can be increased, thereby further improving the synthesis of L-valine. Preferably, the branched-chain amino acid transporter gene brnFE is inserted into the site of pyruvate formate lyase gene pflB of the starting strain. In this way, the L-valine efflux can be enhanced. Preferably, the α-acetolactate synthase gene alsS is inserted into the site of 2,3-butanediol dehydrogenase gene budC of the starting strain. In this way, the synthesis efficiency of α-acetolactate can be further improved, thereby further optimizing the L-valine synthesis flux.

In the case of Enterobacter cloacae, preferably, a gene tandem expression plasmid of the acetohydroxyacid isomeroreductase gene ilvC, the branched-chain amino acid transporter gene brnFE and the α-acetolactate synthase gene alsS is constructed. In this way, the L-valine synthesis flux can be optimized, and the L-valine efflux can be enhanced.

The introducing the target gene coding sequence into the starting strain as described in the present invention includes integrating the target gene coding sequence into the genome of the starting strain or expressing the target gene coding sequence in the starting strain in the form of a plasmid. Depending on the desired expression strength of the inserted gene, a single copy or multiple copies of the coding sequence of the gene can be introduced into the starting strain.

The introduced coding sequences of the genes can be introduced into the starting strain in the forms of respective single gene expression fragments, and also can be introduced into the starting strain in the form of a tandem expression fragment of the gene coding sequence.

In some specific embodiments, the method for constructing an L-valine producing strain of the present invention includes the following steps:
(1) PCR is performed by using the genome of a 2,3-butanediol or acetoin producing strain such as Klebsiella oxytoca PDL-0, Enterobacter cloacae SDM and Bacillus licheniformis 10-1-A as a template to obtain a recombinant fragment containing upstream and downstream homologous sequences of the target gene, the recombinant fragment is ligated to a plasmid pKR6K_{Cm}, pK18mobsacB or pKVM1 and then transferred into strains such as Klebsiella oxytoca PDL-0, Enterobacter cloacae SDM and Bacillus licheniformis 10-1-A by conjugative transfer, and single-exchange transformants are screened by an M9 inorganic salt medium containing 20% citrate in combination with resistance or directly by resistance; and double exchange is conducted under the screening stress of a sucrose lethal gene sacB or temperature-induced screening conditions, and then PCR screening is performed to obtain a strain in which the target gene is knocked out successfully. By this method, genes involved in the formation of by-products (such as acetic acid, formic acid, ethanol, succinic acid and lactic acid) are knocked out from the starting strain.
(2) PCR is performed by using the genome of a 2,3-butanediol or acetoin producing strain such as Klebsiella oxytoca PDL-0, Enterobacter cloacae SDM and Bacillus licheniformis 10-1-A as a template to obtain upstream and downstream homology arm sequences containing a gene such as pox, pta, frdA, ldhD, pflB, adhE, budA, budC or gldA; PCR is performed by using a dihydroxyacid dehydratase plasmid pET28a-puDHT synthesized from a full gene as a template to obtain a puDHT sequence, and the upstream and downstream homology arm sequences of the aforementioned genes and the puDHT sequence are recombined by recombinant PCR to obtain a recombinant fragment, which is ligated to pKR6K_{Cm}, pK18mobsacB and pKVM1, and then transferred into recombinant strains of Klebsiella oxytoca PDL-0, Enterobacter cloacae SDM and Bacillus licheniformis 10-1-A, etc. in which byproduct-related gene knockout has been completed by conjugative transfer, and single-exchange transformants are screened by an M9 inorganic salt medium containing 20% of a citrate in combination with resistance or directly by resistance; and double exchange is conducted under the screening stress of a sucrose lethal gene sacB or temperature-induced screening conditions, and then PCR screening is performed to confirmed that the gene puDHT is successfully inserted into the genomes of recombinant strains of Klebsiella oxytoca PDL-0, Enterobacter cloacae SDM and Bacillus licheniformis 10-1-A, etc. According to the aforementioned gene replacement method, other exogenous genes are inserted into the genomes of recombinant strains of Klebsiella oxytoca PDL-0, Enterobacter cloacae SDM and Bacillus licheniformis 10-1-A in which byproduct-related gene knockout has been completed; or the target gene is expressed by using a plasmid:
(3) PCR is performed by using the genome of Bacillus subtilis 168 as a template to obtain the sequences of an L-leucine dehydrogenase coding gene bcd and an α-acetolactate synthase coding gene alsS; PCR is performed by using the genome of Klebsiella pneumoniae as a template to obtain an α-acetolactate synthase coding gene budB; PCR is performed by using the genome of Escherichia coli W3110 as a template to obtain an acetohydroxyacid isomeroreductase coding gene ilvC, an acetohydroxyacid isomeroreductase cofactor preference mutant protein coding gene ilvC^{M(L67E,R68F,K75E)} and a dihydroxyacid dehydratase coding gene ilvD; whole gene synthesis is conducted to obtain a dihydroxyacid dehydratase coding gene puDHT derived from P. ureilyticus; whole gene synthesis is conducted to obtain a dihydroxyacid dehydratase gene dha) derived from Sulfolobus solfataricus; PCR is performed by using the genome of Corynebacterium glutamicum ATCC13869 as a template to obtain a branched-chain amino acid transporter coding gene brnFE; and PCR is performed by using the genome of Escherichia coli W3110 as a template to obtain a branched-chain amino acid transporter coding gene ygaZH. Preferably, single gene expression fragments of the aforementioned one, preferably two or more, preferably three or more, preferably four or more, preferably five or more, preferably six or more, preferably seven or more, preferably eight or more, and more preferably nine or more genes are obtained, or a gene tandem expression fragment of the aforementioned genes is obtained by recombinant PCR, then connected to linearized expression plasmids pET-28a(+), pET-22b(+), pET25b(+), pETDuet-1, pKD4, pACYCDute and pMMB66EH etc., and then transferred into recombinant strains of Klebsiella oxytoca PDL-0, Enterobacter cloacae SDM, Escherichia coli BL21/pET-RABC, Bacillus licheniformis 10-1-A and Vibrio natriureticus ATCC14048/pET-RABC by electroporation or chemical transformation.

The present invention further provides an L-valine producing strain, which is characterized in that, the strain is constructed by the method according to the present invention,

Preferably, the strain is Klebsiella oxytoca with a deposit number of CCTCC M 20221743. This strain has been deposited in the China Center for Type Culture Collection (CCTCC) on Nov. 9, 2022, at the deposit address: Wuhan University, Wuhan, China, Postal Code: 430072, and the deposit number is: CCTCC M 20221743.

The present invention further provides use of the strain of the present invention in production of L-valine.

By utilizing the L-valine producing strain of the present invention for fermentation, L-valine can be efficiently obtained. Under the conditions of a fermentation volume of 5 liters and a fermentation time of 30-56 hours, the production can reach 45.1-122.0 g/L, the production intensity can reach 1.41-2.18 g/L/h, and the yield can reach 0.246-0.587 g/g.

In a preferred embodiment, the engineered strain of Klebsiella oxytoca of the present invention is utilized to produce L-valine. Under the conditions of a fermentation volume of 5 liters and a fermentation time of 56 hours, the production can reach 122.0 g/L and the yield can reach 0.587 g/g. The engineered strain of Enterobacter cloacae of the present invention is utilized to produce L-valine. Under the conditions of a fermentation volume of 5 liters and a fermentation time of 44 hours, the production of the L-valine can reach 94.3 g/L, the production intensity of the L-valine can reach 2.14 g/L/h, and the yield of the L-valine can reach 0.499 g/g. The engineered strain of Bacillus licheniformis of the present invention is utilized to produce L-valine. Under the conditions of a fermentation volume of 5 liters and a fermentation time of 32 hours, the production of the L-valine can reach 45.1 g/L, the production intensity of the L-valine can reach 1.41 g/L/h, and the yield of the L-valine can reach 0.246 g/g.

The present invention further provides a method for producing L-valine, including the following steps:
1) providing the L-valine producing strain of the present invention;
2) culturing the strain at 30-50° C. for 10-11 hours to provide a seed; and
3) fermenting the seed with glucose as a substrate at 30-50° C., a pH value of 6.0-7.0 and a ventilation volume of 0.5-1.6 vvm to obtain L-valine,

Preferably, in the step 3), the inoculation amount of the seed is an OD₆₂₀nm value reaching 0.2-0.8.

Preferably, the concentration of glucose is 40-60 g/L.

Preferably, the fermentation culture is stirred culture. In fermentation production, the stirring speed can be adjusted according to the dissolved oxygen and is also related to the volume of a fermentation tank. Preferably, the stirring rate is 300-550 revolutions per minute (rpm).

In some specific embodiments of the present invention, fermentation production of L-valine using the Klebsiella oxytoca constructed by the present invention includes the following steps:
(1) Plate culture: the recombinant strain of Klebsiella oxytoca is streaked onto an LB medium containing 1.6-1.8% (w/v) agar and cultured at 37±1° C. for 10±1 hours;
(2) Seed culture: under sterile conditions, a single colony is picked from the plate in the step (1) with a tip of a sterile pipette, then inoculated into 5 mL of an LB liquid medium, and cultured with oscillating in a shaker at 37±1° C. for 10±1 hours; and then inoculated into 50 mL of a LB liquid medium at an inoculation amount of 1% (v/v) and cultured with oscillating in a shaker at 37±1° C. for 10±1 hours;
(3) fermentation tank culture with glucose as a substrate: under sterile conditions, the bacterial solution obtained in the step (2) was taken and inoculated into a fermentation medium containing 50-60 g/L of glucose at an inoculation amount of 10% (v/v). The fermentation conditions are as follows: a culture temperature of 37±1° C., a culture manner of stirring culture, a stirring rotation speed of 500±50 revolutions per minute, a ventilation volume of 1.6±0.1 vvm, a pH maintained at 6.8±0.1 by adjusting with ammonia water, and among the fermentations, samples are taken every 4 hours to detect OD₆₂₀ₙₘ and the concentration of glucose in the fermentation sample. Glucose dry powder is added according to the glucose concentration to maintain the glucose concentration at 40-50 g/L. At the same time, the fermentation samples are subjected to high performance liquid chromatography analysis to determine the concentration of L-valine in the fermentation broth. When glucose is not consumed any more, fermentation is stopped and L-valine is obtained from the fermentation broth.

In some specific embodiments of the present invention, fermentation production of L-valine using the Enterobacter cloacae constructed by the present invention includes the following steps:
(1) Plate culture: the recombinant Enterobacter cloacae strain is streaked onto an LB medium containing agar in a mass-volume ratio of 1.6-1.8% and cultured at 30±1° C. for 10±1 hours;
(2) Seed culture: under sterile conditions, a single colony is picked from the plate in the step (1) with a tip of a sterile pipette, then inoculated into 5 mL of an LB liquid medium, and cultured with oscillating in a shaker at 30±1° C. for 10±1 hours; and then inoculated into 50 mL of a LB liquid medium at an inoculation amount of 1% (v/v) and cultured with oscillating in a shaker at 30±1° C. for 10±1 hours;
(3) fermentation tank culture with glucose as a substrate: under sterile conditions, the bacterial solution obtained in the step (2) was taken and inoculated into a fermentation medium containing 50-60 g/L of glucose at an inoculation amount of 5% (v/v). The fermentation conditions are as follows: a culture temperature of 30±1° C., a culture manner of stirring culture, a stirring rotation speed of 500±50 revolutions per minute, a ventilation volume of 1.0±0.1 vvm, a pH maintained at 7.0±0.1 by adjusting with ammonia water, and among the fermentations, samples are taken every 4 hours to detect OD₆₂₀ₙₘ and the concentration of glucose in the fermentation sample. Glucose dry powder is added according to the glucose concentration to maintain the glucose concentration at 40-50 g/L. At the same time, the fermentation samples are subjected to high performance liquid chromatography analysis to determine the concentration of L-valine in the fermentation broth. When glucose is not consumed any more, fermentation is stopped and L-valine is obtained from the fermentation broth.

In some specific embodiments of the present invention, fermentation production of L-valine using the Bacillus licheniformis constructed by the present invention includes the following steps:
(1) Plate culture: the recombinant Bacillus licheniformis strain is streaked onto an LB medium containing agar in a mass-volume ratio of 1.6-1.8% and cultured at 50±1° C. for 10±1 hours;
(2) Seed culture: under sterile conditions, a single colony is picked from the plate in the step (1) with a tip of a sterile pipette, then inoculated into 5 mL of an LB liquid medium, and cultured with oscillating in a shaker at 50±1° C. for 10±1 hours; and then inoculated into 50 mL of an LB liquid medium at an inoculation amount of 1% (v/v) and cultured with oscillating in a shaker at 50±1° C. for 10±1 hours;
(3) fermentation tank culture with glucose as a substrate: under sterile conditions, the bacterial solution obtained in the step (2) was taken and inoculated into a fermentation medium containing 50-60 g/L of glucose at an inoculation amount of 5% (v/v). The fermentation conditions are as follows: a culture temperature of 50±1° C., a culture manner of stirring culture, a stirring rotation speed of 500±50 revolutions per minute, a ventilation volume of 1.0±0.1 vvm, a pH maintained at 7.0±0.1 by adjusting with ammonia water, and among the fermentations, samples are taken every 4 hours to detect OD₆₂₀ₙₘ and the concentration of glucose in the fermentation sample. Glucose dry powder is added according to the glucose concentration to maintain the glucose concentration at 40-50 g/L. At the same time, the fermentation samples are subjected to high performance liquid chromatography analysis to determine the concentration of L-valine in the fermentation broth. When glucose is not consumed any more, fermentation is stopped and L-valine is obtained from the fermentation broth.

In the aforementioned method for producing L-valine, the fermentation conditions of Klebsiella oxytoca are preferably: a culture temperature of 37° C., an inoculum amount of 10% (v/v), a culture manner of stirring culture, a stirring rotation speed of 500 revolutions per minute, a ventilation volume of 1.6 vvm, and a pH maintained at 6.8 by adjusting with ammonia water; the fermentation conditions of Enterobacter cloacae are preferably: a culture temperature of 30° C., an inoculum amount of 5% (v/v), a culture manner of stirring culture, a stirring rotation speed of 500 revolutions per minute, a ventilation volume of 1.0 vvm, and a pH maintained at 7.0 by adjusting with ammonia water; and the fermentation conditions of Bacillus licheniformis are preferably: a culture temperature of 50° C., an inoculum amount of 5% (v/v), a culture manner of stirring culture, a stirring rotation speed of 500 revolutions per minute, a ventilation volume of 1.0 vvm, and a pH maintained at 7.0 by adjusting with ammonia water.

A method for detecting the substrate glucose during the fermentation process can be carried out employing methods known in the art. For example, after the sample is appropriately diluted, it is measured using a biosensor analyzer SBA-40D (available from Biology Institute of Shandong Academy of Sciences). The determination principle is to utilize the immobilized glucose oxidase membrane to specifically determine the glucose content.

A method for detecting the fermentation product L-valine can be carried out employing methods known in the art. For example, the sample is diluted to an appropriate concentration, and centrifuged at 12,000 rpm for 1 min, and 400 μL of the supernatant is taken, added with 200 μL of a 0.1 M PITC-acetonitrile solution and 200 μL of a 1 M triethylamine-acetonitrile solution, mixed well, and allowed to stand at room temperature with protection from light for 1 h. Then, the mixture is added with 800 μL of n-hexane, vortexed under shaking for 1 min, and allowed to stand at room temperature with protection from light for 10 min. The liquid at the lower layer is taken, filtered through a 0.22 μm filter membrane, and detected for L-valine through a liquid phase. The specific liquid phase detection conditions are as follows:

The model of the used liquid chromatograph is Agilent 1100, and the model of the chromatographic column is ZORBAX SB-C18 (250×4.6 mm, Agilent, USA); the detector is a diode array (UV-Vis) detector; the detection wavelength is 254 nm; the mobile phase A is a 7.6% NaAc-7% acetonitrile aqueous solution (pH 6.5), and the mobile phase B is a 80% acetonitrile aqueous solution. Different proportions of the mobile phases A and B are used for gradient elution, specifically as follows: 0-7% of B for 0-11 min; 7-12% of B for 11-13.9 min; 12-15% of B for 13.9-14 min; 15-34% of B for 14-29 min; 100% of B for 29-37 min; 0% of B for 37-45 min; a flow rate of 0.6 mL/min; a column temperature of 40° C.; an injection volume of 5 μL; and an analysis time of 45 min.

The separation of L-valine from the fermentation broth can be carried out by methods known in the art.

### EXAMPLES

The technical content of the present invention is further described hereafter with reference to examples. The following examples are illustrative, rather than limiting, and cannot limit the claimed scope of the present invention. The experimental methods used in the following examples are conventional methods, unless otherwise specified. The materials, reagents, plasmids, kits, strains, etc. used in the following examples can all be commercially available, unless otherwise specified.

The formula of the M9 inorganic salt medium is: 12.069 g/L of Na₂HPO₄·12H₂O, 3 g/L of KH₂PO₄, 0.5 g/L of NaCl, 0.5 g/L of NH₄Cl, 1 mL of a 1 M MgSO₄ solution, 0.3 mL of a 1 M CaCl₂ solution, 10 mL of a trace element solution (100×), with the pH of the medium being adjusted to 6.8, and the medium being sterilized at 121° C. for 20 minutes. The formula of the trace element solution (100×) is: 5 g/L of EDTA, 0.83 g/L of FeCl₃·6H₂O, 84 mg/L of ZnCl₂, 13 mg/L of CuCl₂·2H₂O, 10 mg/L of CoCl₂·2H₂O, 10 mg/L of H₃BO₃, and 1.6 mg/L of MnCl₂·4H₂O.

The formula of the fermentation medium for Klebsiella oxytoca is as follows: 50-60 g/L of glucose, 5 g/L of yeast powder, 10 g/L of K₂HPO₄, 2 g/L of NaH₂PO₄, 10 g/L of NH₄SO₄, 0.1 g/L of MgSO₄·7H₂O, 1 mL of a 1,000× trace element solution; wherein the formula of the 1,000× trace element solution is: 3.2 g/L of CaCl₂·2H₂O, 3.8 g/L of ZnCl₂, 30 g/L of FeCl₃·2H₂O, 11.14 g/L of MnCl₂·2H₂O, 0.96 g/L of CuCl₂·2H₂O, 2.64 g/L of CoCl₂·2H₂O, 0.35 g/L of H₃BO₃, and 0.024 g/L of NaMoO₄·2H₂O.

The formula of the fermentation medium for Enterobacter cloacae is the aforementioned M9 inorganic salt medium added with 5 g/L of yeast powder and a corresponding concentration of glucose (50-60 g/L).

The formula of the fermentation medium for Bacillus licheniformis is: 50-60 g/L of glucose, 12 g/L of yeast powder, 6.5 g/L of anhydrous sodium acetate (C₂H₃NaO₂), 1 g/L of ammonium citrate (C₆H₁₇N₃0₇), 2 g/L of K₂HPO₄, 0.25 g/L of MgSO₄·7H₂O, 10 mL of a 100× trace element solution; wherein the formula of the 100× trace element solution is: 2.25 g/L of FeSO₄, 0.75 g/L of ZnSO₄, and 0.38 g/L of MnSO₄.

### Example 1: Construction of a L-valine Producing Strain Starting From a 2,3-butanediol Producing Strain Klebsiella oxytoca PDL-0

The starting strain Klebsiella oxytoca PDL-0 was a 2,3-butanediol producing strain obtained by the laboratory of the applicant in the early stage of screening. Its genome had been sequenced and it had been deposited in the China Center for Type Culture Collection on Apr. 15, 2016 with the deposit number: CCTCC NO. M 2016184. K. oxytoca PDL-0 was a gram-negative bacterium that grown aerobically or facultatively anaerobically, with an optimal culture temperature of 37±1° C. K. oxytoca PDL-0 had a positive VP (Voges-Proskauer) reaction and has the ability to metabolize a citrate for growth. The Klebsiella oxytoca had a broad substrate spectrum and could utilize monosaccharides such as glucose, xylose, and galactose as the sole carbon source for growth. It could also metabolize polysaccharides such as cellobiose, cellotriose, and lactose etc. additionally.

### 1.1 Knockout of Byproduct Synthesis-Related Genes

Byproduct synthesis-related genes pox, pta, ldhD, frdA, and pflB were knocked out from the starting strain Klebsiella oxytoca PDL-0.

### 1.1.1 Knockout of the Pyruvate Oxidase Gene Pox

The pyruvate oxidase gene pox had a sequence length of 1,719 bases, and a nucleotide sequence as shown in SEQ ID NO.1.

Construction of knock-out vector: the genomic DNA of Klebsiella oxytoca PDL-0 was prepared by conventional methods. The process referred to the method for small-scale preparation of bacterial genomes in the “Concise Guide to Molecular Biology” published by China Science Publishing & Media Ltd., the genomic DNA of Klebsiella oxytoca PDL-0 was extracted; and the upstream and downstream homology arms of the pox gene were amplified by PCR utilizing the synthetic primers “pox1-f and pox1-r” and “pox2-f and pox2-r”. The obtained upstream and downstream homology arms were used as templates for recombination, and then the recombinant fragment was amplified by PCR utilizing the primers “pox1-f and pox2-r” to obtain a truncated fragment of pox, which contained enzyme cleavage sites of EcoRI and BamHI at both ends.

The truncated recombinant fragment of pox and the suicide plasmid pKR6K_{Cm} were subjected to double enzyme digestion with restriction endonucleases EcoRI and BamHI, respectively. The enzyme-cleaved products were recovered by a nucleic acid gel and ligated with T₄DNA ligase to obtain a knockout plasmid pKR6K_{Cm}-Δpox.

The primers for amplifying the recombinant fragment were designed as follows:

pox1-f:
(SEQ ID NO. 36)
5′-CCGGAATTCACAGACCGTGGCGGCATACA-3′ (EcoRI)
pox1-r:
(SEQ ID NO. 37)
5′-CGCTTACCGTTCATCTGCAAAGCTGGGCCAGCTTTTTCAG-3′
pox2-f:
(SEQ ID NO. 38)
5′-CTGAAAAAGCTGGCCCAGCTTTGCAGATGAACGGTAAGCG-3′
pox2-r:
(SEQ ID NO.39)
5′-CGCGGATCCTTACCTTAGCCAGTTAGTT-3′ (BamHI)

Gene knockout step: Escherichia coli S17-1 λpir carrying a knockout plasmid and Klebsiella oxytoca PDL-0 were inoculated and cultured at 37° C. overnight. The aforementioned strains were transferred and cultured at 37° C. until the OD₆₂₀ₙₘ nm was about 0.6-0.8. 5 mL of a Escherichia coli bacterial solution and 1 mL of a Klebsiella oxytoca bacterial solution were collected respectively, and centrifuged at 6,500 rpm for 3 minutes to collect the bacteria. The bacteria were washed twice with 0.85% normal saline, the two kinds of bacteria were mixed with 100 μL of normal saline, and added dropwise onto an LB plate. The LB plate was placed in a 37° C. incubator for culture overnight. After the biofilm was rinsed with 0.85% normal saline for collection of cells, the rinsing solution was centrifuged at 6,500 rpm for 3 minutes to collect the bacteria, the bacteria was washed twice with 0.85% normal saline, diluted by 4-10 times, spread on a M9 solid plates containing 2% citrate supplemented with chloramphenicol, and cultured at 37° C. for 36-48 hours. The grown single colonies were picked and cultured in an LB medium containing chloramphenicol at 37° C. PCR verification was performed on the bacterial solution by utilizing the upstream and downstream primers to obtain correct single-exchange target bacteria from which both long fragments and short fragments could be obtained through PCR at the same time.

The correct single-exchange target strain was transferred into a resistance-free LB medium, cultured at 37° C. overnight, then transferred into an LB medium containing 15% sucrose and cultured at 37° C. for 10-12 hours. Two generations of transfer were performed, and gradient dilutions of the strain were spread on an LB solid medium containing 15% sucrose and cultured at 37° C. overnight. The grown single colonies were picked and placed in an LB medium. PCR verification was performed on the bacterial solution by utilizing the upstream and downstream primers. The genomes of the single colonies from which only short fragments were amplified were extracted, and subjected to genome temperature gradient PCR verification with the aforementioned primers. Those with all short bands were the correct double-exchange target bacteria.

### 1.1.2 Knockout of the Phosphotransacetylase Gene pta

The phosphotransacetylase gene pta had a sequence length of 2,199 bases, and a nucleotide sequence as shown in SEQ ID NO.2.

The steps of constructing a knockout vector for the gene pta and knocking out the gene referred to the knockout steps of the gene pox in the step 1.1.1 of this example, and the primer sequences were as follows:

pta1-f:
(SEQ ID NO. 40)
5′-CCGGAATTCACTGGCGGTAACGAAAGAGGATA-3′ (EcoRI)
pta1-r:
(SEQ ID NO. 41)
5′-TAAACCTGTTCCGGCAGCACGAAGCTGCTGCGAGTCAG-3′
pta2-f:
(SEQ ID NO. 42)
5′-CTGACTCGCAGCAGCTTCGTGCTGCCGGAACAGGTTTA-3′
pta2-r:
(SEQ ID NO. 43)
5′-TGCTCTAGATTATGCTTGCTGCTGGGACGAC-3′ (XbaI)

1.1.3 Knockout of the Fumarate Reductase Catalytic Subunit Gene frdA

The fumarate reductase catalytic subunit gene frdA had a sequence length of 1,668 bases, and a nucleotide sequence as shown in SEQ ID NO.3.

The steps of constructing a knockout vector for the gene frdA and knocking out the gene referred to the knockout steps of the gene pox in the step 1.1.1 of this example, and the primer sequences were as follows:

frdA1-f:
(SEQ ID NO. 44)
5′-CCGGAATTCATACCGTTGCTGCTGAAGGG-3′ (EcoRI)
frdA1-r:
(SEQ ID NO. 45)
5′-CTTCGCCCAGTTCTCGTTACTGGTATTGTAGCGATACACG-3′
frdA2-f:
(SEQ ID NO. 46)
5′-CGTGTATCGCTACAATACCAGTAACGAGAACTGGGCGAAG-3′
frdA2-r:
(SEQ ID NO. 47)
5′-CGCGGATCCTCAGCCATTCGTCGTCTC-3′ (BamHI)

1.1.4 Knockout of D-Lactate Dehydrogenase Gene ldhD

The D-lactate dehydrogenase gene ldhD had a sequence length of 990 bases, and a nucleotide sequence as shown in SEQ ID NO.4.

The steps of constructing a knockout vector for the gene ldhD and knocking out the gene referred to the knockout steps of the gene pox in the step 1.1.1 of this example, and the primer sequences were as follows:

ldhD1-f:
(SEQ ID NO. 48)
5′-CCGGAATTCTACGAAACAGTACGACAAG-3′ (EcoRI)
ldhD1-r:
(SEQ ID NO. 49)
5′-GAATCGATGAGCGCGCCGCACCGCTTCCGGCGAGTAGGC-3′
ldhD2-f:
(SEQ ID NO. 50)
5′-GAATCGATGAGCGCGCCGCACCGCTTCCGGCGAGTAGGC-3′
ldhD2-r:
(SEQ ID NO. 51)
5′-CGCGGATCCTGACGCAGGTTGTCGAGGGT-3′ (BamHI)

1.1.5 Knockout of the Pyruvate Formate Lyase Gene pflB

The pyruvate formate lyase gene pflB had a sequence length of 2,283 bases, and a nucleotide sequence as shown in SEQ ID NO.5.

The steps of constructing a knockout vector for the gene pflB and knocking out the gene referred to the knockout steps of the gene pox in the step 1.1.1 of this example, and the primer sequences were as follows:

pflB1-f:
(SEQ ID NO. 52)
5′-CCGGAATTCTTAATGAAAAGTTAGCCACA-3′ (EcoRI)
pflB1-r:
(SEQ ID NO. 53)
5′-CGCGAGAGTCGTTGTTACCAGACGGTAGTCACCGATGATA-3′
pflB2-f:
(SEQ ID NO. 54)
5′-TATCATCGGTGACTACCGTCTGGTAACAACGACTCTCGCG-3′
pflB2-r:
(SEQ ID NO. 55)
5′-TGCTCTAGATTACATGGTCTGAGTGAAGG-3′ (Xbal)

Finally, the recombinant Klebsiella oxytoca in which the byproduct-related genes had been correctly knocked out was named Klebsiella oxytoca VKO-0, with the genotype of K. oxytoca PDL-0 Δpox Δpta ΔfrdA ΔldhD ΔpflB.

1.2 Redirection of Metabolic Flux From 2,3-butanediol Synthesis to L-valine Production
1.2.1 Insertion of the Dihydroxyacid Dehydratase Gene puDHT Derived From Paralcaligenes ureilyticus Into the Site of Lactate Dehydrogenase Gene ldhD

The dihydroxyacid dehydratase gene puDHT had a sequence length of 1,728 bases, and a nucleotide sequence as shown in SEQ ID NO.6. The lactate dehydrogenase gene ldhD had a sequence length of 990 bases, and a nucleotide sequence as shown in SEQ ID NO.4.

Construction of gene replacement vector: the genomic DNA of Klebsiella oxytoca PDL-0 was prepared by conventional methods. The process referred to the method for small-scale preparation of bacterial genomes in the “Concise Guide to Molecular Biology” published by China Science Publishing & Media Ltd., and the genomic DNA of Klebsiella oxytoca PDL-0 was extracted. By using the genomic DNA of Klebsiella oxytoca PDL-0 as a template and utilizing primers “ldhD::puDHT1-f and ldhD::puDHT1-r” and “ldhD::puDHT3-f and ldhD::puDHT3-r”, PCR amplification was conducted to replace upstream and downstream homology arms of the gene ldhD. By using the dihydroxyacid dehydratase gene puDHT in the P. ureilyticus obtained through whole gene synthesis as a template and utilizing the primers “ldhD::puDHT2-f and ldhD::puDHT2-r”, the middle segment replacement gene puDHT was obtained through amplification. The obtained upstream homology arm and the gene puDHT were used as templates for recombinant PCR, and then the obtained recombinant fragment and the downstream homology arm were used for recombinant PCR. Thereafter, the recombinant fragment was then amplified by PCR utilizing primers “ldhD::puDHT1-f and ldhD::puDHT3-r” to obtain a gene replacement fragment of ΔldhD:puDHT, which contained enzyme cleavage sites of EcoRI and BamHI at both ends.

The suicide plasmid pKR6K_{Cm} was subjected to double enzyme digestion with restriction endonucleases EcoRI and BamHI. The enzyme-cleaved products were recovered by a nucleic acid gel and ligated with the gene replacement fragment using T5 exonuclease to obtain a gene replacement plasmid pKR6K_{cm}-ΔldhD::puDHT.

The operation steps of inserting the dihydroxyacid dehydratase gene puDHT derived from P. ureilyticus into the site of the lactate dehydrogenase gene ldhD referred to the step of knocking out the gene pox in the step 1.1.1 of Example 1.1.

The primers for amplifying the recombinant fragment were designed as follows:

ldhD::puDHT1-f:
(SEQ ID NO. 56)
5′-AACAGCTATGACATGATTACGAATTCGCCGCTATTGTGGC
ACGTTCGACC-3′ (EcoRI)
ldhD::puDHT1-r:
(SEQ ID NO. 57)
5′-GCTTTTCTTTGTCACTCATAAGACTTTTCTCCAGTGAT-3′
ldhD::puDHT2-f:
(SEQ ID NO. 58)
5′-ATCACTGGAGAAAAGTCTTATGAGTGACAAAGAAAAGC-3′
ldhD::puDHT2-r:
(SEQ ID NO. 59)
5′-GCACAAAAGGGAAAGGAATATTAGTGATTGTCTTTGGGTA-3′
ldhD:puDHT3-f:
(SEQ ID NO. 60)
5′-TACCCAAAGACAATCACTAATATTCCTTTCCCTTTTGTGC-3′
ldhD::puDHT3-r:
(SEQ ID NO. 61)
5′-GCCTGCAGGTCGACTCTAGAGGATCCGTTACTGTCGGCG
TGTAGTAGCAAT-3′ (BamHI)

1.2.2 Replacement of the Ethanol Dehydrogenase Gene adhE With the L-leucine Dehydrogenase Gene bcd Derived From Bacillus subtilis 168

The L-leucine dehydrogenase gene bcd had a sequence length of 1,095 bases, and a nucleotide sequence as shown in SEQ ID NO.7. The ethanol dehydrogenase gene adhE had a sequence length of 2,676 bases, and a nucleotide sequence as shown in SEQ ID NO.8.

The steps for constructing and operating a gene replacement vector for replacing the ethanol dehydrogenase gene adhE with the L-leucine dehydrogenase gene bcd derived from Bacillus subtilis 168 referred to the operation steps of inserting the gene puDHT into the site of the gene ldhD in the step 1.2.1 of this example.

The primers for amplifying the recombinant fragment were designed as follows:

adhE::bcd1-f:
(SEQ ID NO. 62)
5′-AACAGCTATGACATGATTACGAATTCTCAAACAATGATTGAA
TCACAG-3′ (EcoRI)
adhE::bcd1-r:
(SEQ ID NO. 63)
5′-ATATATTTAAAAAGTTCCATAATGCTCTCCTGATAATGTT-3′
adhE::bcd2-f:
(SEQ ID NO. 64)
5′-AACATTATCAGGAGAGCATTATGGAACTTTTTAAATATAT-3′
adhE::bcd2-r:
(SEQ ID NO. 65)
5′-CTGACTTTACGGCTGTGGAATTAACGTCTGCTTAATACAC-3′
adhE::bcd3-f:
(SEQ ID NO. 66)
5′-GTGTATTAAGCAGACGTTAATTCCACAGCCGTAAAGTCAG-3′
adhE::bcd3-r:
(SEQ ID NO. 67)
5′-GCCTGCAGGTCGACTCTAGAGGATCCAGGTGGTGGACCAG
CTCGATATTCC-3′ (BamHI)

1.2.3 Replacement of the α-acetolactate Decarboxylase Gene budA With the Acetohydroxyacid Isomeroreductase Cofactor Preference Mutant Protein Coding Gene ilvC^{M(L67E,R68F,K75E)} Derived From Escherichia coli W3110

The acetohydroxyacid isomeroreductase cofactor preference mutant protein coding gene ilvC^{M(L67E,R68F,K75E)} had a sequence length of 1,476 bases, and a nucleotide sequence as shown in SEQ ID NO.9. The α-acetolactate decarboxylase gene budA had a sequence length of 780 bases, and a nucleotide sequence as shown in SEQ ID NO.10.

Construction of gene replacement vector: the genomic DNA of Klebsiella oxytoca PDL-0 was prepared by conventional methods. The process referred to the method for small-scale preparation of bacterial genomes in the “Concise Guide to Molecular Biology.” published by China Science Publishing & Media Ltd., and the genomic DNA of Klebsiella oxytoca PDL-0 was extracted. The upstream and downstream homology arms of the replacement gene ilvC^{M(L67E,R68F,K75E)} were obtained through amplification by PCR using the genomic DNA of Klebsiella oxytoca PDL-0 as a template and utilizing primers “budA::ilvC^{M(L67E,R68F,K75E)}1-f and budA::ilvC^{M(L67E,R68F,K75E)}1-r” and “budA::ilvC^{M(L67E,R68F,K75E)}4-f and budA::ilvC^{M(L67E,R68F,K75E)}4-r”. By using the genome of Escherichia coli W3110 as a template, the upstream sequence of a mutation site of the middle segment replacement gene ilvC^{M(L67E,R68F,K75E)} was obtained through amplification utilizing primers “budA::ilvC^{M(L67E,R68F,K75E)}2-f and budA::ilvC^{M(L67E,R68F,K75E)}2-r”, the downstream sequence of a mutation site of the middle segment replacement gene ilvC^{M(L67E,R68F,K75E)} was obtained through amplification utilizing primers “budA::ilvC^{M(L67E,R68F,K75E)}3-f and budA::ilvC^{M(L67E,R68F,K75E)}3-r”, and the upstream and downstream sequences were subjected to recombinant PCR to obtain the sequence of the gene ilvC^{M(L67E,R68F,K75E)}. The obtained upstream homology arm and the gene ilvC^{M(L67E,R68F,K75E)} were used as templates for recombinant PCR, and then the obtained recombinant fragment and the downstream homology arm were used for recombinant PCR. Thereafter, the recombinant fragment was then amplified by PCR utilizing primers “budA::ilvC^{M(L67E,R68F,K75E)}1-f and budA::ilvC^{M(L67E,R68F,K75E)}4-r” to obtain a gene replacement fragment of ΔbudA::ilvC^{M(L67E,R68F,K75E)}, which contained enzyme cleavage sites of EcoRI and BamHI at both ends.

The suicide plasmid pKR6K_{Cm} was subjected to double enzyme digestion with restriction endonucleases EcoRI and BamHI. The enzyme-cleaved products were recovered by a nucleic acid gel and ligated with the gene replacement fragment using T5 exonuclease to obtain a gene replacement plasmid pKR6K_{Cm}-ΔbudA::ilvC^{M(L67E,R68F,K75E)}.

The operation steps for replacing the α-acetolactate decarboxylase gene budA with the acetohydroxyacid isomeroreductase cofactor preference mutant protein coding gene ilvC^{M(L67E,R68F,K75E)} derived from Escherichia coli W3110 referred to the operation steps for inserting the gene puDHT into the site of the gene ldhD in step 1.2.1 of this example.

The primers for amplifying the recombinant fragment were designed as follows:

budA::ilvC^{M(L67E,R68F,K75E)}1-f:
(SEQ ID NO. 68)
5′-AACAGCTATGACATGATTACGAATTCTCGCCAT
ATTGCCCTCGACCTG-3′ (EcoRI)
budA::ilvC^{M(L67E,R68F,K75E)}1-r:
(SEQ ID NO. 69)
5′-GTATTGAAGTAGTTAGCCATTACCCGCTTCCTC
GTTCAAC-3′
budA::ilvC^{M(L67E,R68F,K75E)}2-f:
(SEQ ID NO. 70)
5′-GTTGAACGAGGAAGCGGGTAATGGCTAACTAC
TTCAATAC-3′
budA::ilvC^{M(L67E,R68F,K75E)}2-r:
(SEQ ID NO. 71)
5′-GCTTTACGCCAGGACGCGCGCTCCTCGGCAAT
CGCTTCTTTAAACTCAGCGTAGGAGATATCGAGAC-3′
budA::ilvC^{M(L67E,R68F,K75E)}3-f:
(SEQ ID NO. 72)
5′-GTCTCGATATCTCCTACGCTGAGTTTAAAGAAG
CGATTGCCGAGGAGCGCGCGTCCTGGCGTAAAGC-3′
budA::ilvC^{M(L67E,R68F,K75E)}3-r:
(SEQ ID NO. 73)
5′-ATCCACGAGAATCTCCTTAACCCGCAACAGCA
ATAC-3′
budA::ilvC^{M(L67E,R68F,K75E)}4-f:
(SEQ ID NO. 74)
5′-GTATTGCTGTTGCGGGTTAAGGAGATTCTCGTG
GAT-3′
budA::ilvC^{M(L67E,R68F,K75E)}4-r:
(SEQ ID NO. 75)
5′-GCCTGCAGGTCGACTCTAGAGGATCCAGGCGC
TGCCGGGGCGTCCCTGCT-3′ (BamHI)

The final resulting recombinant Klebsiella oxytoca was named Klebsiella oxytoca VKO-3 with the genotype of Klebsiella oxytoca PDL-0 Δpox Δpta ΔfrdA ΔpflB ΔldhD::puDHT ΔadhE::bcd ΔbudA::ilvC^{M(L67E,R68F,K75E)}.

1.3 Introduction of the Branched-Chain Amino Acid Transporter Gene brnFE Derived From Corynebacterium glutamicum ATCC13869 Into the Site of the Pyruvate Formate Lyase Gene pflB to Enhance L-valine Efflux

The branched-chain amino acid transporter gene brnFE had a sequence length of 1,079 bases, and a nucleotide sequence as shown in SEQ ID NO.11. The pyruvate formate lyase gene pflB had a sequence length of 2,283 bases, and a nucleotide sequence as shown in SEQ ID NO.5.

The steps for constructing and operating a gene replacement vector for inserting the branched-chain amino acid transporter gene brnFE derived from Corynebacterium glutamicum ATCC13869 into the site of the pyruvate formate lyase gene pflB referred to the operation steps of inserting the gene puDHT into the site of the gene ldhD in the step 1.2.1 of Example 1.2.

The primers for amplifying the recombinant fragment were designed as follows:

pflB::brnFE1-f:
(SEQ ID NO. 76)
5′-AATTCGAGCTCGGTACCCGGGGATCCAATATATGACTGCCA
ACGGTCAATG-3′ (BamHI)
pflB::brnFE1-r:
(SEQ ID NO. 77)
5′-ATCTCTTGCGTTTTTTGCACGTAACACCTACCTTCTTAA-3′
pflB::brnFE2-f:
(SEQ ID NO. 78)
5′-TTAAGAAGGTAGGTGTTACGTGCAAAAAACGCAAGAGAT-3′
pflB::brnFE2-r:
(SEQ ID NO. 79)
5′-TACGATTTCAGTCAATACCATTAGAAAAGATTCACCAGTC-3′
pflB::brnFE3-f:
(SEQ ID NO. 80)
5′-GACTGGTGAATCTTTTCTAATGGTATTGACTGAAATCGTA-3′
pflB::brnFE3-r:
(SEQ ID NO. 81)
5′-GCCTGCAGGTCGACTCTAGAGGATCCACCTTCTTTCTTAC
AGGCGCGGAAC-3′ (BamHI)

The final resulting recombinant Klebsiella oxytoca was named Klebsiella oxytoca VKO-4 with the genotype of Klebsiella oxytoca PDL-0 Δpox Δpta ΔfrdA ΔldhD::puDHT ΔadhE::bcd ΔbudA::ilvC^{M(L67E,R68F,K75E)} ΔpflB::brnFE.

1.4 Introduction of the α-acetolactate Synthase Gene alsS Derived From Bacillus subtilis 168 to Replace the 2,3-butanediol Dehydrogenase Gene budC to Improve the α-acetolactate Synthesis Efficiency and Inhibit 2,3-butanediol Synthesis

The α-acetolactate synthase gene alsS had a sequence length of 1,713 bases, and a nucleotide sequence as shown in SEQ ID NO.12. The 2,3-butanediol dehydrogenase gene budC had a sequence length of 771 bases, and a nucleotide sequence as shown in SEQ ID NO.13.

The steps for constructing and operating a gene replacement vector for replacing the 2,3-butanediol dehydrogenase gene budC with the α-acetolactate synthase gene alsS derived from Bacillus subtilis 168 referred to the operation steps for inserting the gene puDHT into the site of the gene ldhD in the step 1.2.1 of Example 1.2.

The primers for amplifying the recombinant fragment were designed as follows:

budC::alsS1-f:
(SEQ ID NO. 82)
5′-GAGCTCGGTACCCGGGGATCCGTTTGCCCTTCATCCGCTGC
GCATC-3′ (BamHI)
budC::alsS1-r:
(SEQ ID NO. 83)
5′-TCTTTTGTTGCTTTTGTCAATGCTGGATTCCTTCTGTAGT-3′
budC::alsS2-f:
(SEQ ID NO. 84)
5′-ACTACAGAAGGAATCCAGCATTGACAAAAGCAACAAAAGA-3′
budC::alsS2-r:
(SEQ ID NO. 85)
5′-TGTCAGAGCTTATTTATTACTAGAGAGCTTTCGTTTTCA-3′
budC::alsS3-f:
(SEQ ID NO. 86)
5′-TGAAAACGAAAGCTCTCTAGTAATAAATAAGCTCTGACA-3′
budC::alsS3-r:
(SEQ ID NO. 87)
5′-CAGGTCGACTCTAGAGGATCCGCGGGTCTTTTTGCGCGAGC
TGATC-3′ (BamHI)

The final resulting recombinant Klebsiella oxytoca was named Klebsiella oxytoca VKO-5 with the genotype of Klebsiella oxytoca PDL-0 Δpox Δpta ΔfrdA ΔldhD::puDHT ΔadhE::bcd ΔbudA::ilvC^{M(L67E,R68F,K75E)} ΔpflB::brnFE ΔbudC::alsS.

### 1.5 Increasing the Number of Copies of Acetohydroxyacid Isomeroreductase to Improve L-valine Synthesis

1.5.1 Replacement of the Glycerol Dehydrogenase Gene gldA With the Acetohydroxyacid Isomeroreductase Cofactor Preference Mutant Protein Coding Gene ilvC^{M(L67E,R68F,K75E)} Derived From Escherichia coli W3110

The acetohydroxyacid isomeroreductase cofactor preference mutant protein coding gene ilvC^{M(L67E,R68F,K75E)} had a sequence length of 1,476 bases, and a nucleotide sequence as shown in SEQ ID NO.9. The glycerol dehydrogenase gene gldA had a sequence length of 1,104 bases, and a nucleotide sequence as shown in SEQ ID NO.15.

The steps for constructing and operating a gene replacement vector for replacing the glycerol dehydrogenase gene gldA with the acetohydroxyacid isomeroreductase cofactor preference mutant protein coding gene ilvC^{M(L67E,R68F,K75E)} derived from Escherichia coli W3110 referred to the operation steps of inserting the gene puDHT into the site of the gene ldhD in the step 1.2.1 of Example 1.2.

The primers for amplifying the recombinant fragment were designed as follows:

gldA::ilvC^{M(L67E,R68F,K75E)}1-f:
(SEQ ID NO. 88)
5′-GAGCTCGGTACCCGGGGATCCGCCGTATTCTTT
CCTCAACGACACT-3′ (BamHI)
gldA::ilvC^{M(L67E,R68F,K75E)}1-r:
(SEQ ID NO. 89)
5′-GTATTGAAGTAGTTAGCCATTTCTATTCCTCCTG
GATGACCGTG-3′
gldA::ilvC^{M(L67E,R68F,K75E)}2-f:
(SEQ ID NO. 90)
5′-CACGGTCATCCAGGAGGAATAGAAATGGCTAA
CTACTTCAATAC-3′
gldA::ilvCM^{(L67E,R68F,K75E)}2-r:
(SEQ ID NO. 91)
5′-CTGCTGGCGGGTATGTCGCGAGGGGTTAACCC
GCAACAGCAATAC-3′
gldA::ilvC^{M(L67E,R68F,K75E)}3-f:
(SEQ ID NO. 92)
5′-GTATTGCTGTTGCGGGTTAACCCCTCGCGACAT
ACCCGCCAGCAG-3′
gldA::ilvC^{M(L67E,R68F,K75E)}3-r:
(SEQ ID NO. 93)
5′-CAGGTCGACTCTAGAGGATCCATAACGAGGTC
AAGGTCTGCCAGGC-3′ (BamHI)

The final resulting recombinant Klebsiella oxytoca was named Klebsiella oxytoca VKO-6 with the genotype of Klebsiella oxytoca PDL-0 Δpox Δpta ΔfrdA ΔldhD::puDHT ΔadhE::bcd ΔbudA::ilvC^{M(L67E,R68F,K75E)} ΔpflB::brnFE ΔbudC::alsS ΔgldA::ilvC^{M(L67E,R68F,K75E)}.

1.5.2 Replacement of the Glycerol Dehydrogenase Gene gldA With the Acetohydroxyacid Isomeroreductase Coding Gene ilvC Derived From Escherichia coli W3110

The acetohydroxyacid isomeroreductase gene ilvC had a sequence length of 1,476 bases, and a nucleotide sequence as shown in SEQ ID NO.14.

The steps for constructing and operating a gene replacement vector for replacing the glycerol dehydrogenase gene gldA with the acetohydroxyacid isomeroreductase gene ilvC derived from Escherichia coli W3110 referred to the operation steps of inserting the gene puDHT into the site of the gene ldhD in the step 1.2.1 of Example 1.2.

The primers for amplifying the recombinant fragment were designed as follows:

gldA::ilvC1-f:
(SEQ ID NO. 94)
5′-GAGCTCGGTACCCGGGGATCCGCCGTATTCTTTCCTCAACG
ACACT-3′ (BamHI)
gldA::ilvC1-r:
(SEQ ID NO. 95)
5′-GTATTGAAGTAGTTAGCCATTTCTATTCCTCCTGGATGACCG
TG-3′
gldA::ilvC2-f:
(SEQ ID NO. 96)
5′-CACGGTCATCCAGGAGGAATAGAAATGGCTAACTACTTCAA
TAC-3′
gldA::ilvC2-r:
(SEQ ID NO. 97)
5′-CTGCTGGCGGGTATGTCGCGAGGGGTTAACCCGCAACAGCA
ATAC-3′
gldA::ilvC3-f:
(SEQ ID NO. 98)
5′-GTATTGCTGTTGCGGGTTAACCCCTCGCGACATACCCGCCAG
CAG-3′
gldA::ilvC3-r:
(SEQ ID NO. 99)
5′-CAGGTCGACTCTAGAGGATCCATAACGAGGTCAAGGTCTGC
CAGGC-3′ (BamHI)

The final resulting recombinant Klebsiella oxytoca was named Klebsiella oxytoca VKO-7 with the genotype of Klebsiella oxytoca PDL-0 Δpox Δpta ΔfrdA ΔldhD::puDHT ΔadhE::bcd ΔbudA::ilvC^{M(L67E,R68F,K75E)} ΔpflB::brnFE ΔbudC::alsS ΔgldA::ilvC.

### 1.6 Optimizing the Source of Dihydroxy Acid Dehydratase to Improve Synthesis of L-valine

1.6.1 Replacement of the Dihydroxyacid Dehydratase Gene puDHT Derived From P. ureilyticus With the Dihydroxyacid Dehydratase Gene dhal Derived From Sulfolobus solfataricus

The dihydroxyacid dehydratase gene dhaD had a sequence length of 1,677 bases, and a nucleotide sequence as shown in SEQ ID NO.16.

The steps for constructing and operating a gene replacement vector for replacing the dihydroxyacid dehydratase gene puDHT derived from P. ureilyticus with the dihydroxyacid dehydratase gene dhaD derived from Sulfolobus solfataricus referred to the operation steps of inserting the gene puDHT into the site of the gene ldhD in the step 1.2.1 of Example 1.2.

The primers for amplifying the recombinant fragment were designed as follows:

puTHD::dhaD1-f:
(SEQ ID NO. 100)
5′-AACAGCTATGACATGATTACGAATTCCTGTGAGTTAAAG
TTTCCATCCCG-3′ (EcoRI)
puTHD::dhaD1-r:
(SEQ ID NO. 101)
5′-GAGTTCAGTTTTGCCGGCATAAGACTTTTCTCCAGTGAT
A-3′
puTHD::dhaD2-f:
(SEQ ID NO. 102)
5′-TATCACTGGAGAAAAGTCTTATGCCGGCAAAACTGAAC
TC-3′
puTHD::dhaD2-r:
(SEQ ID NO. 103)
5′-GCACAAAAGGGAAAGGAATATTATGCCGGACGCGTAAC
CGCGC-3′
puTHD::dhaD3-f:
(SEQ ID NO. 104)
5′-GCGCGGTTACGCGTCCGGCATAATATTCCTTTCCCTTTTG
TGC-3′
puTHD::dhaD3-r:
(SEQ ID NO. 105)
5′-GCCTGCAGGTCGACTCTAGAGGATCCTGTAGTAGCAATG
ATGAACCTGTTC-3′ (BamHI)

The final resulting recombinant Klebsiella oxytoca was named Klebsiella oxytoca VKO-8 with the genotype of Klebsiella oxytoca PDL-0 Δpox Δpta ΔfrdA ΔldhD::dhaD ΔadhE::bcd ΔbudA::ilvC^{M(L67E,R68F,K75E)} ΔpflB::brnFE ΔbudC::alsS ΔgldA::ilvC.

1.6.2 Replacement of the Dihydroxyacid Dehydratase Gene puDHT Derived From P. ureilyticus With the Dihydroxyacid Dehydratase Gene ilvD Derived From Escherichia coli W3110

The dihydroxyacid dehydratase gene ilvD had a sequence length of 1,851 bases, and a nucleotide sequence as shown in SEQ ID NO.17.

The steps for constructing and operating a gene replacement vector for replacing the dihydroxyacid dehydratase gene puDHT derived from P. ureilyticus with the dihydroxyacid dehydratase gene ilvD derived from Escherichia coli W3110 referred to the operation steps of inserting the gene puDHT into the site of the gene ldhD in the step 1.2.1 of Example 1.2.

The primers for amplifying the recombinant fragment were designed as follows:

puTHD::ilvD1-f:
(SEQ ID NO. 106)
5′-AACAGCTATGACATGATTACGAATTCGGCACGTTCGACCT
GTGAGTTAAA-3′ (EcoRI)
puTHD::ilvD1-r:
(SEQ ID NO. 107)
5′-GCGGAACGGTACTTAGGCATAAGACTTTTCTCCAGTGATA-
3′
puTHD::ilvD2-f:
(SEQ ID NO. 108)
5′-TATCACTGGAGAAAAGTCTTATGCCTAAGTACCGTTCCGC-
3′
puTHD::ilvD2-r:
(SEQ ID NO. 109)
5′-GCACAAAAGGGAAAGGAATATTAACCCCCCAGTTTCGA-3′
puTHD::ilvD3-f:
(SEQ ID NO. 110)
5′-TCGAAACTGGGGGGTTAATATTCCTTTCCCTTTTGTGC-3′
puTHD:ilvD3-r:
(SEQ ID NO. 111)
5′-GCCTGCAGGTCGACTCTAGAGGATCCTCGGCGTGTAGTA
GCAATGATGAAC-3′ (BamHI)

The final resulting recombinant Klebsiella oxytoca obtained by construction was named Klebsiella oxytoca VKO-9 with the genotype of Klebsiella oxytoca PDL-0 Δpox Δpta ΔfrdA ΔldhD::ilvD ΔadhE::bcd ΔbudA::ilvC^{M(L67E,R68F,K75E)} ΔpflB::brnFE ΔbudC::alsS ΔgldA:ilvC.

### Example 2: Batch Fermentation of L-valine Using Glucose as a Substrate by Recombinant Klebsiella oxytoca

2.1 Batch Fermentation of L-valine Using Glucose as a Substrate by Recombinant Klebsiella oxytoca VKO-3
(1) Plate culture: the recombinant strain Klebsiella oxytoca VKO-3 was streaked onto an LB medium containing agar at a mass-volume ratio of 1.6-1.8%, and cultured at 37±1° C. for 10±1 h;
(2) Seed culture: under sterile conditions, a single colony was picked from the plate in the step (1) with a tip of a sterile pipette, then inoculated into 5 mL of an LB liquid medium, and cultured with oscillating in a shaker at 37±1° C. for 10±1 hours; and then inoculated into 100 mL of an LB liquid medium at an inoculation amount of 1% (v/v) and cultured with oscillating in a shaker at 37±1° C. for 10±1 hours;
(3) 1 L fermentation tank culture: under sterile conditions, the bacterial solution obtained in the step (2) was taken and inoculated into a fermentation medium containing 50-60 g/L of glucose at an inoculation amount of 10% (v/v). The fermentation conditions were as follows: a liquid loading volume of 0.8 L, a culture temperature of 37±1° C., a culture manner of stirring culture, a stirring rotation speed of 500±50 revolutions per minute, a ventilation volume of 1.6±0.1 vvm, and a pH maintained at 6.8±0.1 by adjusting with ammonia water, and among the fermentations, samples were taken every 4 hours to detect OD₆₂₀ₙₘ and the concentration of the glucose in the fermentation sample; and at the same time the fermentation sample was subjected to high performance liquid chromatography analysis to determine the concentration of L-valine in the fermentation broth. When the glucose was consumed completely, the fermentation was stopped.

In the aforementioned method, a method for detecting the substrate glucose was: appropriately diluting the sample and then determining the diluted sample using a biosensor analyzer SBA-40D (available from Biology Institute of Shandong Academy of Sciences). The determination principle was to utilize the immobilized glucose oxidase membrane to specifically determine the glucose content.

In the aforementioned method, a method for detecting the fermentation product L-valine was that:
the sample was diluted to an appropriate concentration, and centrifuged at 12,000 rpm for 1 min, and 400 μL of the supernatant was taken, added with 200 μL of a 0.1 M PITC-acetonitrile solution and 200 μL of a 1 M triethylamine-acetonitrile solution, mixed well, and allowed to stand at room temperature with protection from light for 1 h. Then, the mixture was added with 800 μL of n-hexane, vortexed under shaking for 1 min, and then allowed to stand at room temperature with protection from light for 10 min. The liquid at the lower layer was taken, filtered through a 0.22 μm filter membrane, and detected for L-valine through a liquid phase. The specific liquid phase detection conditions were as follows:

The model of the used liquid chromatograph was Agilent 1100, and the model of the chromatographic column was ZORBAX SB-C18 (250×4.6 mm, Agilent, USA); the detector was a diode array (UV-Vis) detector; the detection wavelength was 254 nm; the mobile phase A was a 7.6% NaAc-7% acetonitrile aqueous solution (pH 6.5), and the mobile phase B was a 80% acetonitrile aqueous solution. Different proportions of the mobile phases A and B were used for gradient elution, specifically as follows: 0-7% of B for 0-11 min; 7-12% of B for 11-13.9 min; 12-15% of B for 13.9-14 min; 15-34% of B for 14-29 min; 100% of B for 29-37 min; 0% of B for 37-45 min; a flow rate of 0.6 mL/min; a column temperature of 40° C.; an injection volume of 5 μL; and an analysis time of 45 min.

The results showed that the recombinant strain K. oxytoca VKO-3 was cultured for 28 h, which consumed 60.0 g/L of glucose, and the concentration of L-valine reached 6.39 g/L, and the yield of L-valine reached 0.107 g/g.

The formula of the LB medium described in the aforementioned steps (1)-(2) was: 10 g/L of peptone; 5 g/L of yeast powder; 10 g/L of NaCl, at pH 7.0; sterilized at 121° C. for 20 minutes.

The formula of the fermentation medium described in the aforementioned step (3) was: 5 g/L of yeast powder, 10 g/L of K₂HPO₄, 2 g/L of KH₂PO₄, 10 g/L of (NH₄)₂SO₄, 0.1 g/L of MgSO₄·7H₂O, 1 mL of a trace element solution (1,000×), with the pH of the medium being adjusted to 6.8, and the medium being sterilized at 121° C. for 20 minutes. The formula of the trace element solution (1,000×) was: 3.2 g/L of CaCl₂·2H₂O, 3.8 g/L of ZnCl₂, 30 g/L of FeCl₃·2H₂O, 11.14 g/L of MnCl₂·2H₂O, 0.96 g/L of CuCl₂·2H₂O, 2.64 g/L of CoCl₂·2H₂O, 0.35 g/L of H₃BO₃, and 0.024 g/L of NaMoO₄·2H₂O.

2.2 Batch Fermentation of L-valine Using Glucose as a Substrate by Recombinant Klebsiella oxytoca VKO-4

The method and operation steps for batch fermentation of L-valine using glucose as a substrate by a recombinant strain Klebsiella oxytoca VKO-4 referred to the method and operation steps in the step 2.1 of this example.

The results showed that the recombinant strain Klebsiella oxytoca VKO-4 was cultured for 20 h, which consumed 60.0 g/L of glucose, and the concentration of L-valine reached 11.0 g/L, and the yield of L-valine reached 0.183 g/g.

2.3 Batch Fermentation of L-valine Using Glucose as a Substrate by Recombinant Klebsiella oxytoca VKO-5

The method and operation steps for batch fermentation of L-valine using glucose as a substrate by a recombinant strain Klebsiella oxytoca VKO-5 referred to the method and operation steps in the step 2.1 of this example.

The results showed that the recombinant strain Klebsiella oxytoca VKO-5 was cultured for 24 h, which consumed 60.0 g/L of glucose, and the concentration of L-valine reached 10.6 g/L, and the yield of L-valine reached 0.177 g/g.

2.4 Batch Fermentation of L-valine Using Glucose as a Substrate by Recombinant Klebsiella oxytoca VKO-6

The method and operation steps for batch fermentation of L-valine using glucose as a substrate by a recombinant strain Klebsiella oxytoca VKO-6 referred to the method and operation steps in the step 2.1 of this example.

The results showed that the recombinant strain Klebsiella oxytoca VKO-6 was cultured for 24 h, which consumed 60.0 g/L of glucose, and the concentration of L-valine reached 10.2 g/L, and the yield of L-valine reached 0.170 g/g.

2.5 Batch Fermentation of L-valine Using Glucose as a Substrate by Recombinant Klebsiella oxytoca VKO-7

The method and operation steps for batch fermentation of L-valine using glucose as a substrate by a recombinant strain Klebsiella oxytoca VKO-7 referred to the method and operation steps in the step 2.1 of this example.

The results showed that the recombinant strain Klebsiella oxytoca VKO-7 was cultured for 24 h, which consumed 60.0 g/L of glucose, and the concentration of L-valine reached 21.5 g/L, and the yield of L-valine reached 0.358 g/g.

2.6 Batch Fermentation of L-valine Using Glucose as a Substrate by Recombinant Klebsiella oxytoca VKO-8

The method and operation steps for batch fermentation of L-valine using glucose as a substrate by a recombinant strain Klebsiella oxytoca VKO-8 referred to the method and operation steps in the step 2.1 of this example.

The results showed that the recombinant strain Klebsiella oxytoca VKO-8 was cultured for 28 h, which consumed 59.0 g/L of glucose, and the concentration of L-valine reached 23.2 g/L, and the yield of L-valine reached 0.393 g/g.

2.7 Batch Fermentation of L-valine Using Glucose as a Substrate by Recombinant Klebsiella oxytoca VKO-9

The method and operation steps for batch fermentation of L-valine using glucose as a substrate by a recombinant strain Klebsiella oxytoca VKO-9 referred to the method and operation steps in the step 2.1 of this example.

The results showed that the recombinant strain Klebsiella oxytoca VKO-9 was cultured for 20 h, which consumed 59.0 g/L of glucose, and the concentration of L-valine reached 35.5 g/L, and the yield of L-valine reached 0.602 g/g.

### Example 3: Fed-Batch Fermentation of L-valine Using Glucose as a Substrate by Recombinant Klebsiella oxytoca VKO-9

(1) Plate culture: the recombinant strain Klebsiella oxytoca VKO-9 was streaked onto an LB medium containing agar at a mass-volume ratio of 1.6-1.8%, and cultured at 37±1° C. for 10±1 h;
(2) Seed culture: under sterile conditions, a single colony was picked from the plate in the step (1) with a tip of a sterile pipette, then inoculated into 5 mL of an LB liquid medium, and cultured with oscillating in a shaker at 37±1° C. for 10±1 hours; and then inoculated into 100 mL of an LB liquid medium at an inoculation amount of 1% (v/v) and cultured with oscillating in a shaker at 37±1° C. for 10±1 hours;
(3) 7.5 L fermentation tank culture: under sterile conditions, the bacterial solution obtained in the step (2) was taken and inoculated into a fermentation medium containing 50-60 g/L of glucose at an inoculation amount of 10% (v/v). The fermentation conditions were as follows: a liquid loading volume of 5 L, a culture temperature of 37±1° C., a culture manner of stirring culture, a stirring rotation speed of 500±50 revolutions per minute, a ventilation volume of 1.6±0.1 vvm, and a pH maintained at 6.8±0.1 by adjusting with ammonia water, and among the fermentations, samples were taken every 4 hours to detect OD₆₂₀ₙₘ and the concentration of the glucose in the fermentation sample, and glucose powder was added according to the glucose concentration to maintain the glucose concentration at 40-50 g/L; and at the same time the fermentation sample was subjected to high performance liquid chromatography analysis to determine the concentration of L-valine in the fermentation broth. When glucose was not consumed any more, fermentation was stopped and L-valine was obtained from the fermentation broth.

The results showed that the recombinant strain K. oxytoca VKO-9 was cultured for 56 h, which consumed 208.0 g/L of glucose, and the concentration of L-valine reached 122.0 g/L, the production intensity reached 2.18 g/L/h, and the yield of L-valine reached 0.587 g/g.

The methods for detecting the substrate glucose and the product L-valine as well as the formula of the LB medium and the formula of the fermentation medium described in the aforementioned steps were consistent with those in Example 2.

### Example 4: Construction of an L-valine Producing Strain Starting From the 2,3-butanediol Producing Strain Enterobacter cloacae SDM

The starting strain Enterobacter cloacae SDM was a 2,3-butanediol producing strain obtained by the laboratory of the applicant in the early stage of screening. Its genome had been sequenced and it had been deposited in the China General Microbiological Culture Collection Center on Oct. 19, 2010, with the deposit number CGMCC No. 4230. The Enterobacter cloacae SDM belonged to the genus Enterobacter of the family Enterobacteriaceae, and was a facultative anaerobic gram-negative bacterium. The Enterobacter cloacae could produce acid and gas by fermenting glucose, was positive in a VP reaction, and has the ability to metabolize a citrate for growth. Enterobacter cloacae had a broad substrate spectrum and could metabolize monosaccharides such as arabinose, xylose and galactose, as well as polysaccharides such as cellobiose, sucrose and lactose.

### 4.1 Knockout of Byproduct Synthesis-Related Genes

In the starting strain Enterobacter cloacae SDM, the byproduct synthesis-related genes pox_{Ec}, pta_{Ec}, ldhD_{Ec}, frdA_{Ec} pflB_{Ec}, adhE_{Ec}, budA_{Ec}, budC_{Ec} and gldA_{Ec} were knocked out.

4.1.1 Knockout of the Pyruvate Oxidase Gene pox_{Ec}

The pyruvate oxidase gene pox_{Ec} had a sequence length of 978 bases, and a nucleotide sequence as shown in SEQ ID NO.18.

Construction of knockout vector: the genomic DNA of Enterobacter cloacae SDM was prepared by conventional methods. The process referred to the method for small-scale preparation of bacterial genomes in the “Concise Guide to Molecular Biology” published by China Science Publishing & Media Ltd., the genomic DNA of Enterobacter cloacae SDM was extracted; and the upstream and downstream homology arms of the gene pox_{Ec} were amplified by PCR utilizing the synthetic primers “pox_{Ec}1-ff and pox_{Ec}1-r” and “pox_{Ec}2-f and pox_{Ec}2-r”. The obtained upstream and downstream homology arms were used as templates for recombination, and then the recombinant fragment was amplified by PCR utilizing the primers “pox_{Ec}1-f and pox_{Ec}2-r” to obtain a truncated fragment of pox_{Ec}, which contained enzyme cleavage sites of EcoRI and BamHI at both ends.

The truncated recombinant fragment of pox_{Ec} and the suicide plasmid pK18mobsacB were subjected to double enzyme digestion with restriction endonucleases EcoRI and BamHI, respectively. The enzyme-cleaved products were recovered by a nucleic acid gel and ligated with T₄DNA ligase to obtain a knockout plasmid pK18mobsacB-Δpox_{Ec}.

The steps for knocking out the gene pox_{Ec} referred to the knockout step of gene pox in the step 1.1.1 of Example 1, and the primer sequences were as follows:

pox_{Ec}1-f:
(SEQ ID NO. 112)
5′-CCGGAATTCTCGAAGCTGTCATGATCCTG-3′ (EcoRI)
pox_{Ec}1-r:
(SEQ ID NO. 113)
5′-GGGGAGAGGGGGTACTGCCGGGGTAATTCTCCGATTTCAG-3′
pox_{Ec}2-f:
(SEQ ID NO. 114)
5′-CTGAAATCGGAGAATTACCCCGGCAGTACCCCCTCTCCCC-3′
pox_{Ec}2-r:
(SEQ ID NO. 115)
5′-CGCGGATCCCACCACGATCAGCGACTGGGATCGC-3′
(BamHI)

4.1.2 Knockout of the Phosphotransacetylase Gene pta_{Ec}

The phosphotransacetylase gene pta_{Ec} had a sequence length of 2,142 bases, and a nucleotide sequence as shown in SEQ ID NO.19.

The steps of constructing a knockout vector for the gene pta_{Ec} and knocking out the gene referred to the knockout steps of the gene pox_{Ec} in the step 4.1.1 of this example, and the primer sequences were as follows:

pta_{Ec}1-f:
(SEQ ID NO. 116)
5′-CCGGAATTCCATGAGCGTTGACCAGATCA-3′ (EcoRI)
pta_{Ec}1-r:
(SEQ ID NO. 117)
5′-GCCATCCGGCAAGACCTTATGGTTTATCCTCTTTCGTTAC-3′
pta_{Ec}2-f:
(SEQ ID NO. 118)
5′-GTAACGAAAGAGGATAAACCATAAGGTCTTGCCGGATGGC-3′
pta_{Ec}2-r:
(SEQ ID NO. 119)
5′-TGCTCTAGAAAGGCAATGTGCTGCGCGAAGGAAG-3′ (XbaI)

4.1.3 Knockout of Fumarate Reductase Subunit A Gene frdA_{Ec}

The fumarate reductase catalytic subunit gene frdA_{Ec} had a sequence length of 1,791 bases, and a nucleotide sequence as shown in SEQ ID NO.20.

The steps of constructing a knockout vector for the gene frdA_{Ec} and knocking out the gene referred to the knockout steps of the gene pox_{Ec} in the step 4.1.1 of this example, and the primer sequences were as follows:

frdA_{Ec}1-f:
(SEQ ID NO. 120)
5′-CCGGAATTCCCGGGGCCAACAAAACGGGT-3′ (EcoRI)
frdA_{Ec}1-r:
(SEQ ID NO. 121)
5′-CAACTTTCAGGGTTTGCATCGACATTCCTCCAGATTTTTG-3′
frdA_{Ec}2-f:
(SEQ ID NO. 122)
5′-CAAAAATCTGGAGGAATGTCGATGCAAACCCTGAAAGTTG-3′
frdA_{Ec}2-r:
(SEQ ID NO. 123)
5′-CGCGGATCCCGATGAACTCAGGGTTCAGACCAAA-3′ (BamHI)

4.1.4 Knockout of D-lactate Dehydrogenase Gene ldhD_{Ec}

The D-lactate dehydrogenase gene ldhD_{Ec} had a sequence length of 990 bases, and a nucleotide sequence as shown in SEQ ID NO.21.

The steps of constructing a knockout vector for the gene ldhD_{Ec} and knocking out the gene referred to the knockout steps of the gene pox_{Ec} in the step 4.1.1 of this example, and the primer sequences were as follows:

ldhD_{Ec}1-f:
(SEQ ID NO. 124)
5′-CCGGAATTCACCGTGTTAAGTTCAAGCGC-3′ (EcoRI)
ldhD_{Ec}1-r:
(SEQ ID NO. 125)
5′-CCGCCACCCGGCATGTCGGCAAGACTTTCTCCAGTGATTT-3′
ldhD_{Ec}2-f:
(SEQ ID NO. 126)
5′-AAATCACTGGAGAAAGTCTTGCCGACATGCCGGGTGGCGG-3′
ldhD_{Ec}2-r:
(SEQ ID NO. 127)
5′-CGCGGATCCGGCGACGGTCATTATTTCGCAGGCG-3′
(BamHI)

4.1.5 Knockout of the Pyruvate Formate Lyase Gene pflB_{Ec}

The pyruvate formate lyase gene pflB_{Ec} had a sequence length of 2,283 bases, and a nucleotide sequence as shown in SEQ ID NO.22.

The steps of constructing a knockout vector for the gene pflB_{Ec} and knocking out the gene referred to the knockout steps of the gene pox_{Ec} in the step 4.1.1 of this example, and the primer sequences were as follows:

pflB_{Ec}1-f:
(SEQ ID NO. 128)
5′-CCGGAATTCAGTATATGACCGCCAACGGC-3′ (EcoRI)
pflB_{Ec}1-r:
(SEQ ID NO. 129)
5′-GTGATTTCAGTCAATTCCAGGTAACACCTACCTTCTTAAG-3′
pflB_{Ec}2-f:
(SEQ ID NO. 130)
5′-CTTAAGAAGGTAGGTGTTACCTGGAATTGACTGAAATCAC-3′
pflB_{Ec}2-r:
(SEQ ID NO. 131)
5′-TGCTCTAGATGTATGCCTTCTTTGTGGCAGGCAC-3′ (XbaI)

4.1.6 Knockout of Ethanol Dehydrogenase Gene adhE_{Ec}

The ethanol dehydrogenase gene adhE_{Ec} had a sequence length of 1,002 bases, and a nucleotide sequence as shown in SEQ ID NO.23.

The steps of constructing a knockout vector for the gene adhE_{Ec} and knocking out the gene referred to the knockout steps of the gene pox_{Ec} in the step 4.1.1 of this example, and the primer sequences were as follows:

adhE_{Ec}1-f:
(SEQ ID NO. 132)
5′-CCGGAATTCTCGTCAGAAATCGAGACATC-3′ (EcoRI)
adhE_{Ec}1-r:
(SEQ ID NO. 133)
5′-GGGTGAGGGAATCAGGCCACGGTGAACTCCTCAATGGAAT-3′
adhE_{Ec}2-f:
(SEQ ID NO. 134)
5′-ATTCCATTGAGGAGTTCACCGTGGCCTGATTCCCTCACCC-3′
adhE_{Ec}2-r:
(SEQ ID NO. 135)
5′-TGCTCTAGATTCAGTATTCTGATTACGATAAAAT-3′ (XbaI)

4.1.7 Knockout of α-acetolactate Decarboxylase Gene budA_{Ec}

The α-acetolactate decarboxylase gene budA_{Ec} had a sequence length of 780 bases, and a nucleotide sequence as shown in SEQ ID NO.24.

The steps of constructing a knockout vector for the gene budA_{Ec} and knocking out the gene referred to the knockout steps of the gene pox_{Ec} in the step 4.1.1 of this example, and the primer sequences were as follows:

budA_{Ec}1-f:
(SEQ ID NO. 136)
5′-CCGGAATTCGAAGACATATTGGCCTCCAC-3′ (EcoRI)
budA_{Ec}1-r:
(SEQ ID NO. 137)
5′-TGTTCACGGTAGTTCTCCTGCATGCTCGTCCTCTTCAACT-3′
budA_{Ec}2-f:
(SEQ ID NO. 138)
5′-AGTTGAAGAGGACGAGCATGCAGGAGAACTACCGTGAACA-3′
budA_{Ec}2-r:
(SEQ ID NO. 139)
5′-TGCTCTAGACACCGCCCGGCCTGCCGTGCTCGGC-3′ (XbaI)

4.1.8 Knockout of 2,3-butanediol Dehydrogenase Gene budC_{Ec}

The 2,3-butanediol dehydrogenase gene budC_{Ec} had a sequence length of 771 bases, and a nucleotide sequence as shown in SEQ ID NO.25.

The steps of constructing a knockout vector for the gene budC_{Ec} and knocking out the gene referred to the knockout steps of the gene pox_{Ec} in the step 4.1.1 of this example, and the primer sequences were as follows:

budC_{Ec}1-f:
(SEQ ID NO. 140)
5′-CCGGAATTCATCGCCCGCTACCTCTACAG-3′ (EcoRI)
budC_{Ec}1-r:
(SEQ ID NO. 141)
5′-ATGTCAGAGCTTATTAGAATTTCTCTGTCCTTATAGTGAG-3′
budC_{Ec}2-f:
(SEQ ID NO. 142)
5′-CTCACTATAAGGACAGAGAAATTCTAATAAGCTCTGACAT-3′
budC_{Ec}2-r:
(SEQ ID NO. 143)
5′-TGCTCTAGATTCGCCCGGCTTTTTGTCGGATTTC-3′ (XbaI)

4.1.9 Knockout of Glycerol Dehydrogenase Gene gldA_{Ec}

The glycerol dehydrogenase gene gldA_{Ec} had a sequence length of 1,104 bases, and a nucleotide sequence as shown in SEQ ID NO.26.

The steps of constructing a knockout vector for the gene gldA_{Ec} and knocking out the gene referred to the knockout steps of the gene pox_{Ec} in the step 4.1.1 of this example, and the primer sequences were as follows:

gldA_{Ec}1-f:
(SEQ ID NO. 144)
5′-CCGGAATTCAAACAATGAGCCGCGACGCA-3′ (EcoRI)
gldA_{Ec}1-r:
(SEQ ID NO. 145)
5′-TAGCGCACCCGGCGTTTTTGAACATATCTCCCTTAGAGGT-3′
gldA_{Ec}2-f:
(SEQ ID NO. 146)
5′-ACCTCTAAGGGAGATATGTTCAAAAACGCCGGGTGCGCTA-3′
gldA_{Ec}2-r:
(SEQ ID NO. 147)
5′-TGCTCTAGACGATTGCCGACGGTTTCCGCAACTA-3′ (XbaI)

Finally, the engineered strain of Enterobacter cloacae in which the byproduct-related genes had been correctly knocked out was named Enterobacter cloacae VEC-0, with the genotype of Enterobacter cloacae SDM Δpox_{Ec} Δpta_{Ec} ΔfrdA_{Ec} ΔldhD_{Ec} ΔpflB_{Ec} ΔadhE_{Ec} ΔbudA_{Ec} ΔbudC_{Ec} ΔgldA_{Ec}.

4.2 Redirection of Metabolic Flux From 2,3-butanediol Synthesis to L-valine Production
4.2.1 Insertion of Dihydroxyacid Dehydratase Gene ilvD Derived From Escherichia coli W3110 Into the Site of D-lactate Dehydrogenase Gene ldhD_{Ec}

The dihydroxyacid dehydratase gene ilvD had a sequence length of 1,851 bases, and a nucleotide sequence as shown in SEQ ID NO.17.

The steps for constructing and operating a gene replacement vector for inserting the dihydroxyacid dehydratase gene ilvD derived from Escherichia coli W3110 into the site of D-lactate dehydrogenase gene ldhD_{Ec} referred to the operation steps of inserting the gene puDHT into the site of the gene ldhD in the step 1.2.1 of Example 1.

The primers for amplifying the recombinant fragment were designed as follows:

ldhD_{Ec}::ilvD1-f:
(SEQ ID NO. 148)
5′-AACAGCTATGACATGATTACGAATTCACCGTGTTAAGTTC
AAGCGC-3′ (EcoRI)
ldhD_{Ec}::ilvD1-r:
(SEQ ID NO. 149)
5′-GCGGAACGGTACTTAGGCATAAGACTTTCTCCAGTGATTT-
3′
ldhD_{Ec}::ilvD2-f:
(SEQ ID NO. 150)
5′-AAATCACTGGAGAAAGTCTTATGCCTAAGTACCGTTCCGC-
3′
ldhD_{Ec}::ilvD2-r:
(SEQ ID NO. 151)
5′-CCGCCACCCGGCATGTCGGCTTAACCCCCCAGTTTCGATT-
3′
ldhD_{Ec}::ilvD3-f:
(SEQ ID NO. 152)
5′-AATCGAAACTGGGGGGTTAAGCCGACATGCCGGGTGGCG
G-3′
ldhD_{Ec}::ilvD3-r:
(SEQ ID NO. 153)
5′-GCCTGCAGGTCGACTCTAGAGGATCCGGCGACGGTCATTA
TTTCGCAGGCG-3′ (BamHI)

4.2.2 Insertion of the L-leucine Dehydrogenase Gene bcd Derived From Bacillus subtilis 168 Into the Site of Ethanol Dehydrogenase Gene adhE_{Ec}

The L-leucine dehydrogenase gene bcd had a sequence length of 1,095 bases, and a nucleotide sequence as shown in SEQ ID NO.7.

The steps for constructing and operating a gene replacement vector for inserting the L-leucine dehydrogenase gene bcd derived from Bacillus subtilis 168 into the site of ethanol dehydrogenase gene adhE_{Ec} referred to the operation steps of inserting the gene puDHT into the site of the gene ldhD in the step 1.2.1 of Example 1.

The primers for amplifying the recombinant fragment were designed as follows:

adhE_{Ec}::bcd1-f:
(SEQ ID NO. 154)
5′-AACAGCTATGACATGATTACGAATTCTCGTCAGAAATCGA
GACATC-3′ (EcoRI)
adhE_{Ec}::bcd1-r:
(SEQ ID NO. 155)
5′-ATATATTTAAAAAGTTCCATGGTGAACTCCTCAATGGAAT-3′
adhE_{Ec}::bcd2-f:
(SEQ ID NO. 156)
5′-ATTCCATTGAGGAGTTCACCATGGAACTTTTTAAATATAT-3′
adhE_{Ec}::bcd2-r:
(SEQ ID NO. 157)
5′-GGGTGAGGGAATCAGGCCACTTAACGTCTGCTTAATACAC-
3′
adhE_{Ec}::bcd3-f:
(SEQ ID NO. 158)
5′-GTGTATTAAGCAGACGTTAAGTGGCCTGATTCCCTCACCC-
3′
adhE_{Ec}::bcd3-r:
(SEQ ID NO. 159)
5′-GCCTGCAGGTCGACTCTAGAGGATCCTTCAGTATTCTGATT
ACGATAAAAT-3′ (BamHI)

4.2.3 Insertion of the Acetohydroxyacid Isomeroreductase Cofactor Preference Mutant Protein Coding Gene ilvC^{M(L67E,R68F,K75E)} Derived From Escherichia coli W3110 Into the Site of α-Acetolactate Decarboxylase Gene budA_{Ec}

The acetohydroxyacid isomeroreductase cofactor preference mutant protein coding gene ilvC^{M(L67E,R68F,K73E)} had a sequence length of 1,476 bases, and a nucleotide sequence as shown in SEQ ID NO.9.

The steps for constructing and operating a gene replacement vector for inserting the acetohydroxyacid isomeroreductase cofactor preference mutant protein coding gene ilvC^{M(L67E,R68F,K73E)} derived from Escherichia coli W3110 into the site of α-acetolactate decarboxylase gene budA_{Ec} referred to the operation steps of replacing the gene budA with the gene ilvC^{M(L67E,R68F,K73E)} in the step 1.2.3 of Example 1.

The primers for amplifying the recombinant fragment were designed as follows:

budA_{Ec}::ilvC^{M(L67E,R68F,K75E)}1-f:
(SEQ ID NO. 160)
5′-AACAGCTATGACATGATTACGAATTCGAAGA
CATATTGGCCTCCAC-3′ (EcoRI)
budA_{Ec}::ilvC^{M(L67E,R68F,K75E)}1-r:
(SEQ ID NO. 161)
5′-GTATTGAAGTAGTTAGCCATCATGCTCGTCCT
CTTCAACT-3′
budA_{Ec}::ilvC^{M(L67E,R68F,K75E)}2-f:
(SEQ ID NO. 162)
5′-AGTTGAAGAGGACGAGCATGATGGCTAACTA
CTTCAATAC-3′
budA_{Ec}::ilvC^{M(L67E,R68F,K75E)}2-r:
(SEQ ID NO. 163)
5′-GCTTTACGCCAGGACGCGCGCTCCTCGGCAA
TCGCTTCTTTAAACTCAGCGTAGGAGATATCGAGAC-3′
budA_{Ec}::ilvC^{M(L67E,R68F,K75E)}3-f:
(SEQ ID NO. 164)
5′-GTCTCGATATCTCCTACGCTGAGTTTAAAGAA
GCGATTGCCGAGGAGCGCGCGTCCTGGCGTAAAGC-3′
budA_{Ec}::ilvC^{M(L67E,R68F,K75E)}3-r:
(SEQ ID NO. 165)
5′-TGTTCACGGTAGTTCTCCTGTTAACCCGCAA
CAGCAATAC-3′
budA_{Ec}::ilvC^{M(L67E,R68F,K75E)}4-f:
(SEQ ID NO. 166)
5′-GTATTGCTGTTGCGGGTTAACAGGAGAACTA
CCGTGAACA-3′
budA_{Ec}::ilvC^{M(L67E,R68F,K75E)}4-r:
(SEQ ID NO. 167)
5′-GCCTGCAGGTCGACTCTAGAGGATCCCACCG
CCCGGCCTGCCGTGCTCGGC-3′ (BamHI)

The final resulting recombinant engineered strain of Enterobacter cloacae was named Enterobacter cloacae VEC-3, with the genotype of Enterobacter cloacae SDM Δpox_{Ec} Δpta_{Ec} ΔfrdA_{Ec} ΔpflB_{Ec} ΔbudC_{Ec} ΔgldA_{Ec} ΔldhD_{Ec}::ilvD ΔadhE_{Ec}::bcd ΔbudA_{Ec}::ilvC^{M(L67E,R68F,K73E)}

4.3 Plasmid Overexpression of the Acetohydroxyacid Isomeroreductase Coding Gene ilvC Derived from Escherichia coli W3110, the Branched-Chain Amino Acid Transporter Gene brnFE Derived From Corynebacterium glutamicum ATCC13869, and the α-acetolactate Synthase Gene alsS Derived From Bacillus subtilis 168 to Improve L-valine Synthesis

The branched-chain amino acid transporter gene brnFE had a sequence length of 1,079 bases, and a nucleotide sequence as shown in SEQ ID NO.11. The α-acetolactate synthase gene alsS had a sequence length of 1,713 bases, and a nucleotide sequence as shown in SEQ ID NO.12. The acetohydroxyacid isomeroreductase gene ilvC had a sequence length of 1,476 bases, and a nucleotide sequence as shown in SEQ ID NO.14.

Construction of gene expression vector: the genome of Escherichia coli W3110 was used as a template, and the synthetic primers “ilvC-f and ilvC-r” were utilized for PCR amplification to obtain a ilvC gene fragment; the genome of Corynebacterium glutamicum ATCC13869 was used as a template, and the synthetic primers “brnFE-f and brnFE-r” were utilized for PCR amplification to obtain a brnFE gene fragment; and the genome of Bacillus subtilis 168 was used as a template, and the synthetic primers “alsS-f and alsS-r” were utilized for PCR amplification to obtain an alsS gene fragment. The obtained gene fragments of ilvC, brnFE and alsS were used as templates for recombinant PCR, and then the recombinant fragments were amplified by PCR using the primers “ilvC-f and alsS-r” to obtain a gene tandem expression fragment of ilvC, brnFE and alsS, which contained enzyme cleavage sites of EcoRI and BamHI at both ends.

The gene tandem expression fragments of ilvC, brnFE and alsS and pKD4 were subjected to double enzyme digestion with restriction endonucleases EcoRI and BamHI, respectively. The enzyme-cleaved products were recovered by a nucleic acid gel and ligated with T₄DNA ligase to obtain a gene expression plasmid pKD4-ilvC-brnFE-alsS.

Introduction of expression plasmid by electroporation: the recombinant strain Enterobacter cloacae VEC-3 was transferred into a shake flask in an LB medium containing 0.7 mM of EDTA, cultured until the OD₆₂₀nm was about 0.6-0.8, and placed on ice for 30 min. The culture solution was centrifuged at 6,000 rpm for 8 min to collect strains, the strains were washed three times with sterile ddH₂O, and resuspended to an OD₆₂₀ₙₘ of about 50, which was the competent cell. 100 μL of the competent cells were taken, added with about 1 μg of the plasmid to be transformed, tapped to mix well, placed on ice for 5 min, and added into a 2 mm electroporation cup. Electroporation was conducted with electroporation parameters of 2,000 V, 200 Ω, and 25 μF. Immediately after electroporation, 900 μL of an LB liquid medium was added. Then the culture solution was transferred into a sterile centrifuge tube, incubated in a shaker at 37° C. and 180 rpm for 1 h, diluted and spread on a resistant LB solid medium containing 50 μg/mL of kanamycin, and incubated in a 37° C. incubator overnight.

The primers for amplifying the gene tandem expression fragment were designed as follows:

ilvC-f:
(SEQ ID NO. 168)
5′-AGCGAATTCATGGCTAACTACTTCAATAC-3′ (EcoRI)
ilvC-r:
(SEQ ID NO. 169)
5′-ATCTCTTGCGTTTTTTGCACTTAACCCGCAACAGCAATAC-3′
brnFE-f:
(SEQ ID NO. 170)
5′-GTATTGCTGTTGCGGGTTAAGTGCAAAAAACGCAAGAGAT-3′
brnFE-r:
(SEQ ID NO. 171)
5′-CTTTTGTTGCTTTTGTCAATTAGAAAAGATTCACCAGTC-3′
alsS-f:
(SEQ ID NO. 172)
5′-GACTGGTGAATCTTTTCTAATTGACAAAAGCAACAAAAGA-3′
alsS-r:
(SEQ ID NO. 173)
5′-AGCGGATCCCTAGAGAGCTTTCGTTTTCA-3′
(BamHI)

The final resulting recombinant engineered strain of Enterobacter cloacae was named Enterobacter cloacae VEC-3/pKD4-ilvC-brnFE-alsS, with the genotype of Enterobacter cloacae SDM Δpox_{Ec} Δpta_{Ec} ΔfrdA_{Ec} ΔpflB_{Ec} ΔbudC_{Ec} ΔgldA_{Ec} ΔldhD_{Ec}::ilvD ΔadhE_{Ec}::bcd ΔbudA_{Ec}::ilvC^{M(L67E,R68F,K75E)}/pKD4-ilvC-brnFE-alsS.

4.4 Fed-Batch Fermentation of Engineered Strain of Enterobacter cloacae VEC-3/pKD4-ilvC-brnFE-alsS Using Glucose as a Substrate to Produce L-valine
(1) Plate culture: the recombinant Enterobacter cloacae strain VEC-3/pKD4-ilvC-brnFE-alsS was streaked onto an LB medium containing agar in a mass volume ratio of 1.6-1.8% and 50 μg/mL of kanamycin, and cultured at 30±1° C. for 10±1 h.
(2) Seed culture: under sterile conditions, a single colony was picked from the plate in the step (1) with a tip of a sterile pipette, then inoculated into 5 mL of an LB liquid medium containing 50 μg/mL of kanamycin, and cultured with oscillating in a shaker at 30±1° C. for 10±1 hours; and then inoculated into 100 mL of an LB liquid medium containing 50 μg/mL of kanamycin at an inoculation amount of 1% (v/v) and cultured with oscillating in a shaker at 30±1° C. for 10±1 hours;
(3) 7.5 L fermentation tank culture: under sterile conditions, the bacterial solution obtained in the step (2) was taken and inoculated into a fermentation medium containing 50-60 g/L of glucose and 50 μg/mL of kanamycin at an inoculation amount of 5% (v/v). The fermentation conditions were as follows: a liquid loading volume of 5 L, a culture temperature of 30±1° C., a culture manner of stirring culture, a stirring rotation speed of 500±50 revolutions per minute, a ventilation volume of 1.0±0.1 vvm, and a pH maintained at 7.0±0.1 by adjusting with ammonia water, and among the fermentations, samples were taken every 4 hours to detect OD₆₂₀ₙₘ and the concentration of the glucose in the fermentation sample, and glucose powder was added according to the glucose concentration to maintain the glucose concentration at 40-50 g/L; and at the same time the fermentation sample was subjected to high performance liquid chromatography analysis to determine the concentration of L-valine in the fermentation broth. When glucose was not consumed any more, fermentation was stopped and L-valine was obtained from the fermentation broth.

The results showed that the recombinant strain VEC-3/pKD4-ilvC-brnFE-alsS was cultured for 44 h, which consumed 189.0 g/L of glucose, and the concentration of L-valine reached 94.3 g/L, the production intensity reached 2.14 g/L/h, and the yield of L-valine reached 0.499 g/g.

The methods for detecting the substrate glucose and the product L-valine as well as the formula of the LB medium described in the aforementioned steps were consistent with those in Example 2.

The formula of the fermentation medium described in the aforementioned step (3) was: 5 g/L of yeast powder, 12.069 g/L of Na₂HPO₄·12H₂O, 3 g/L of KH₂PO₄, 0.5 g/L of NaCl, 0.5 g/L of NH₄Cl, 1 mL of a 1 M MgSO4 solution, 0.3 mL of a 1 M CaCl₂ solution, 10 mL of a trace element solution (100×), with the pH of the medium being adjusted to 6.8, and the medium being sterilized at 121° C. for 20 minutes. The formula of the trace element solution (100×) is: 5 g/L of EDTA, 0.83 g/L of FeCl₃·6H₂O, 84 mg/L of ZnCl₂, 13 mg/L of CuCl₂·2H₂O, 10 mg/L of CoCl₂·2H₂O, 10 mg/L of H₃BO₃, and 1.6 mg/L of MnCl₂·4H₂O.

### Example 5: Construction of an L-valine Producing Strain Starting From the 2,3-butanediol Producing Strain Bacillus licheniformis 10-1-A.

The starting strain Bacillus licheniformis 10-1-A was deposited in the China General Microbiological Culture Collection Center on Nov. 14, 2011, with the deposit number: CGMCC NO. 5461. The Bacillus licheniformis 10-1-A belonged to a gram-positive bacterium that grows aerobically or facultatively anaerobically, had strong protein secretion ability and a fast growth rate, and was a recognized biosafety bacterium. It had a rod-shaped morphology, a red or white colony color, produced spores, had a positive VP reaction, could utilize glucose, sucrose, and fructose to produce acids, could hydrolyze casein, gelatin, and Tween 80, could utilize a citrate, could grow in a medium containing 100 g/L of NaCl, and could grow under conditions of 42-60° C.

### 5.1 Knockout of Byproduct Synthesis-Related Genes

In the starting strain Bacillus licheniformis 10-1-A, the byproduct synthesis-related genes pox_{Bl}, pta_{Bl}, ldhD_{Bl}, frdA_{Bl}, pflB_{Bl}, adhE_{Bl}, budA_{Bl}, budC_{Bl} and gldA_{Bl} were knocked out.

5.1.1 Knockout of the Pyruvate Oxidase Gene pox_{Bl}

The pyruvate oxidase gene pox_{Bl} had a sequence length of 1,722 bases, and a nucleotide sequence as shown in SEQ ID NO.27.

Construction of knockout vector: the genomic DNA of Bacillus licheniformis 10-1-A was prepared by conventional methods. The process referred to the method for small-scale preparation of bacterial genomes in the “Concise Guide to Molecular Biology” published by China Science Publishing & Media Ltd., the genomic DNA of Bacillus licheniformis 10-1-A was extracted; and the upstream and downstream homology arms of the pox_{Bl} gene were amplified by PCR utilizing the synthetic primers “pox_{Bl}1-f and pox_{Bl}1-r” and “pox_{Bl}2-f and pox_{Bl}2-r”. The obtained upstream and downstream homology arms were used as templates for recombination, and then the recombinant fragment was amplified by PCR utilizing the primers “pox_{Bl}1-f and pox_{Bl}2-r” to obtain a truncated fragment of pox_{Bl}, which contained enzyme cleavage sites of SmaI and BamHI at both ends.

The truncated recombinant fragment of pox_{Bl} and the suicide plasmid pKVM1 were subjected to double enzyme digestion with restriction endonucleases SmaI and BamHI, respectively. The enzyme-cleaved products were recovered by a nucleic acid gel and ligated with T₄DNA ligase to obtain a knockout plasmid pKVM1-Δpox_{Bl}.

The steps for knocking out the gene pox_{Ec} referred to the knockout step of gene pox in the step 1.1.1 of Example 1, and the primer sequences were as follows:

pox_{Bl}1-f:
(SEQ ID NO. 174)
5′-CCGGGGCCCGTACCTGTCGCGGGTGTGAC-3′
(SmaI)
pox_{Bl}1-r:
(SEQ ID NO. 175)
5′-CGATCTCCCTTGGCATCATAATCACGTCCTCCTTTGTTTT-3′
pox_{Bl}2-f:
(SEQ ID NO. 176)
5′-AAAACAAAGGAGGACGTGATTATGATGCCAAGGGAGATCG-3′
pox_{Bl}2-r:
(SEQ ID NO. 177)
5′-CGCGGATCCGGGGGCGGTATATGTCCAGGTAAAG-3′
(BamHI)

5.1.2 Knockout of the Phosphotransacetylase Gene pta_{Bl}

The phosphotransacetylase gene pta_{Bl} had a sequence length of 972 bases, and a nucleotide sequence as shown in SEQ ID NO.28.

The steps of constructing a knockout vector for the gene pta_{Bl} and knocking out the gene referred to the knockout steps of the gene pox_{Bl} in the step 5.1.1 of this example, and the primer sequences were as follows:

pta_{Bl}1-f:
(SEQ ID NO. 178)
5′-CCGGGGCCCCGACGATGCTGTAAAGCGTA-3′
(SmaI)
pta_{Bl}1-r:
(SEQ ID NO. 179)
5′-CACCTTTTTCAGGAAGCCTATATATACCCTCCTTGAAAGT-3′
pta_{Bl}2-f:
(SEQ ID NO. 180)
5′-ACTTTCAAGGAGGGTATATATAGGCTTCCTGAAAAAGGTG-3′
pta_{Bl}2-r:
(SEQ ID NO. 181)
5′-TGCGGATCCAAGAAAAGCGATTATCTTTATACAT-3′
(BamHI)

5.1.3 Knockout of Fumarate Reductase Subunit A Gene frdA_{Bl}

The fumarate reductase catalytic subunit gene frdA_{Bl} had a sequence length of 1,389 bases, and a nucleotide sequence as shown in SEQ ID NO.29.

The steps of constructing a knockout vector for the gene frdA_{Bl} and knocking out the gene referred to the knockout steps of the gene pox_{Bl} in the step 5.1.1 of this example, and the primer sequences were as follows:

frdA_{Bl}1-f:
(SEQ ID NO. 182)
5′-CCGGGGCCCAAAGTAAAAAATGATTCCGT-3′
(SmaI)
frdA_{Bl}1-r:
(SEQ ID NO. 183)
5′-GCATGCAGCCGGTTTGTTGATTTCTTATCCCTTCCTTCTC-3′
frdA_{Bl}2-f:
(SEQ ID NO. 184)
5′-GAGAAGGAAGGGATAAGAAATCAACAAACCGGCTGCATGC-3′
frdA_{Bl}2-r:
(SEQ ID NO. 185)
5′-CGCGGATCCAAGAAAAGCGATTATCTTTATACAT-3′
(BamHI)

5.1.4 Knockout of L-lactate Dehydrogenase Gene ldhL_{Bl}

The L-lactate dehydrogenase gene ldhL_{Bl} had a sequence length of 960 bases, and a nucleotide sequence as shown in SEQ ID NO.30.

The steps of constructing a knockout vector for the gene ldhL_{Bl} and knocking out the gene referred to the knockout steps of the gene pox_{Bl} in the step 5.1.1 of this example, and the primer sequences were as follows:

ldhL_{Bl}1-f:
(SEQ ID NO. 186)
5′-CCGGGGCCCTATAAAAAAGATGACAACAA-3′
(SmaI)
ldhL_{Bl}1-r:
(SEQ ID NO. 187)
5′-AGTATCTTCATGGTGTTCAGGACTCATCATTCCTTTGCCG-3′
ldhL_{Bl}2-f:
(SEQ ID NO. 188)
5′-CGGCAAAGGAATGATGAGTCCTGAACACCATGAAGATACT-3′
ldhL_{Bl}2-r:
(SEQ ID NO. 189)
5′-CGCGGATCCGTTTAAAACCAAGCTCGACAAGAAG-3′
(BamHI)

5.1.5 Knockout of the Pyruvate Formate Lyase Gene pflB_{Bl}

The pyruvate formate lyase gene pflB_{Bl} had a sequence length of 2,226 bases, and a nucleotide sequence as shown in SEQ ID NO.31.

The steps of constructing a knockout vector for the gene pflB_{Bl} and knocking out the gene referred to the knockout steps of the gene pox_{Bl} in the step 5.1.1 of this example, and the primer sequences were as follows:

pflB_{Bl}1-f:
(SEQ ID NO. 190)
5′-CCGGGGCCCTGACTTCTCCCATTGCAGCA-3′
(SmaI)
pflB_{Bl}1-r:
(SEQ ID NO. 191)
5′-CGCGCTCCGCTTATTGCTCGTTAAATCCCCCTCTTTTTCA-3′
pflB_{Bl}2-f:
(SEQ ID NO. 192)
5′-TGAAAAAGAGGGGGATTTAACGAGCAATAAGCGGAGCGCG-3′
pflB_{Bl}2-r:
(SEQ ID NO. 193)
5′-TGCGGATCCGGCATTCCTGTCAGGTTGATATGTT-3′
(BamHI)

5.1.6 Knockout of Ethanol Dehydrogenase Gene adhE_{Bl}

The ethanol dehydrogenase gene adhE_{Bl} had a sequence length of 2,604 bases, and a nucleotide sequence as shown in SEQ ID NO.32.

The steps of constructing a knockout vector for the gene adhE_{Bl} and knocking out the gene referred to the knockout steps of the gene pox_{Bl} in the step 5.1.1 of this example, and the primer sequences were as follows:

adhE_{Bl}1-f:
(SEQ ID NO. 194)
5′-CCGGGGCCCTCGCTGAAAAACTAAAAGAA-3′
(SmaI)
adhE_{Bl}1-r:
(SEQ ID NO. 195)
5′-CGGAATGACGGCTTTTTTGGTGTAAACCCTCCAGTGAATG-3′
adhE_{Bl}2-f:
(SEQ ID NO. 196)
5′-CATTCACTGGAGGGTTTACACCAAAAAAGCCGTCATTCCG-3′
adhE_{Bl}2-r:
(SEQ ID NO. 197)
5′-TGCGGATCCTGCGAATGGTTGTACTTCTTTTCCG-3′
(BamHI)

5.1.7 Knockout of α-acetolactate Decarboxylase Gene budA_{Bl}

The α-acetolactate decarboxylase gene budA_{Bl} had a sequence length of 762 bases, and a nucleotide sequence as shown in SEQ ID NO.33.

The steps of constructing a knockout vector for the gene budA_{Bl} and knocking out the gene referred to the knockout steps of the gene pox_{Bl} in the step 5.1.1 of this example, and the primer sequences were as follows:

budA_{Bl}1-f:
(SEQ ID NO. 198)
5′-CCGGGGCCCTGGGGTGGCTTTGCCGTGGG-3′
(SmaI)
budA_{Bl}1-r:
(SEQ ID NO. 199)
5′-TTCAAAGAGGGCTTTTTCATTTTCCTCTTTTCACTCCCTT-3′
budA_{Bl}2-f:
(SEQ ID NO. 200)
5′-AAGGGAGTGAAAAGAGGAAAATGAAAAAGCCCTCTTTGAA-3′
budA_{Bl}2-r:
(SEQ ID NO. 201)
5′-TGCGGATCCTTGAAGCGATCAGAAGCTCAGGGAA-3′
(BamHI)

5.1.8 Knockout of 2,3-butanediol Dehydrogenase Gene budC_{Bl}

The 2,3-butanediol dehydrogenase gene budC_{Bl} had a sequence length of 783 bases, and a nucleotide sequence as shown in SEQ ID NO.34.

The steps of constructing a knockout vector for the gene budC_{Bl} and knocking out the gene referred to the knockout steps of the gene pox_{Bl} in the step 5.1.1 of this example, and the primer sequences were as follows:

budC_{Bl}1-f:
(SEQ ID NO. 202)
5′-CCGGGGCCCAAAGCGCATGTTTTAAAACC-3′
(SmaI)
budC_{Bl}1-r:
(SEQ ID NO. 203)
5′-TATAGAATATAATTTTAAAAATAAACATCTTCTTTCTATA-3′
budC_{Bl}2-f:
(SEQ ID NO. 204)
5′-TATAGAAAGAAGATGTTTATTTTTAAAATTATATTCTATA-3′
budC_{Bl}2-r:
(SEQ ID NO. 205)
5′-TGCGGATCCTTGAAGCGATCAGAAGCTCAGGGAA-3′
(BamHI)

5.1.9 Knockout of Glycerol Dehydrogenase Gene gldA_{Bl}

The glycerol dehydrogenase gene gldA_{Bl} had a sequence length of 1,104 bases, and a nucleotide sequence as shown in SEQ ID NO.35.

The steps of constructing a knockout vector for the gene gldA_{Bl} and knocking out the gene referred to the knockout steps of the gene pox_{Bl} in the step 5.1.1 of this example, and the primer sequences were as follows:

gldA_{Bl}1-f:
(SEQ ID NO. 206)
5′-ATTTAGATCTAACAAGCCGCGTCATTCAAG-3′
(BglII)
gldA_{Bl}1-r:
(SEQ ID NO. 207)
5′-ACTTGGCGCCATTCTTCTTCGACACATCGCAAATGATA-3′
gldA_{Bl}2-f:
(SEQ ID NO. 208)
5′-TATCATTTGCGATGTGTCGAAGAAGAATGGCGCCAAGT-3′
gldA_{Bl}2-r:
(SEQ ID NO. 209)
5′-TACCGTGGATCCGCTTTAAG-3′
(BamHI)

Finally, the recombinant Bacillus licheniformis in which the byproduct-related genes had been correctly knocked out was named Bacillus licheniformis VBL-0, with the genotype of Bacillus licheniformis 10-1-A Δpox_{Bl} Δpta_{Bl} ΔfrdA_{Bl} ΔldhL_{Bl} ΔpflB_{Bl} ΔadhE_{bl} ΔbudA_{Bl} ΔbudC_{Bl} ΔgldA_{Bl}.

5.2 Redirection of Metabolic Flux From 2,3-butanediol Synthesis to L-valine Production
5.2.1 Insertion of Dihydroxyacid Dehydratase Gene ilvD Derived From Escherichia coli W3110 Into the Site of L-lactate Dehydrogenase Gene ldhL_{Bl}

The dihydroxyacid dehydratase gene ilvD had a sequence length of 1,851 bases, and a nucleotide sequence as shown in SEQ ID NO.17.

The steps for constructing and operating a gene replacement vector for inserting the dihydroxyacid dehydratase gene ilvD derived from Escherichia coli W3110 into the site of L-lactate dehydrogenase gene ldhL_{Bl} referred to the operation steps of inserting the gene puDHT into the site of the gene ldhD in the step 1.2.1 of Example 1.

The primers for amplifying the recombinant fragment were designed as follows:

ldhL_{Bl}::ilvD1-f:
(SEQ ID NO. 210)
5′-CCGGGGCCCTATAAAAAAGATGACAACAA-3′
(SmaI)
ldhL_{Bl}::ilvD1-r:
(SEQ ID NO. 211)
5′-GCGGAACGGTACTTAGGCATGACTCATCATTCCTTTGCCG-3′
ldhL_{Bl}::ilvD2-f:
(SEQ ID NO. 212)
5′-CGGCAAAGGAATGATGAGTCATGCCTAAGTACCGTTCCGC-3′
ldhL_{Bl}::ilvD2-r:
(SEQ ID NO. 213)
5′-AGTATCTTCATGGTGTTCAGTTAACCCCCCAGTTTCGATT-3′
ldhL_{Bl}::ilvD3-f:
(SEQ ID NO. 214)
5′-AATCGAAACTGGGGGGTTAACTGAACACCATGAAGATACT-3′
ldhL_{Bl}::ilvD3-r:
(SEQ ID NO. 215)
5′-CGCGGATCCGTTTAAAACCAAGCTCGACAAGAAG-3′
(BamHI)

5.2.2 Insertion of the L-leucine Dehydrogenase Gene bcd Derived From Bacillus subtilis 168 Into the Site of Ethanol Dehydrogenase Gene adhE_{Bl}

The L-leucine dehydrogenase gene bcd had a sequence length of 1,095 bases, and a nucleotide sequence as shown in SEQ ID NO.7.

The steps for constructing and operating a gene replacement vector for inserting the L-leucine dehydrogenase gene bcd derived from Bacillus subtilis 168 into the site of ethanol dehydrogenase gene adhE_{Bl} referred to the operation steps of inserting the gene puDHT into the site of the gene ldhD in the step 1.2.1 of Example 1.

The primers for amplifying the recombinant fragment were designed as follows:

adhE_{Bl}::bcd1-f:
(SEQ ID NO. 216)
5′-CCGGGGCCCTCGCTGAAAAACTAAAAGAA-3′
(SmaI)
adhE_{Bl}::bcd1-r:
(SEQ ID NO. 217)
5′-ATATATTTAAAAAGTTCCATTGTAAACCCTCCAGTGAATG-3′
adhE_{Bl}::bcd2-f:
(SEQ ID NO. 218)
5′-CATTCACTGGAGGGTTTACAATGGAACTTTTTAAATATAT-3′
adhE_{Bl}::bcd2-r:
(SEQ ID NO. 219)
5′-CGGAATGACGGCTTTTTTGGTTAACGTCTGCTTAATACAC-3′
adhE_{Bl}::bcd3-f:
(SEQ ID NO. 220)
5′-GTGTATTAAGCAGACGTTAACCAAAAAAGCCGTCATTCCG-3′
adhE_{Bl}::bcd3-r:
(SEQ ID NO. 221)
5′-TGCGGATCCTGCGAATGGTTGTACTTCTTTTCCG-3′
(BamHI)

5.2.3 Insertion of the Acetohydroxyacid Isomeroreductase Cofactor Preference Mutant Protein Coding Gene ilvC^{M(L67E,R68F,K75E)} Derived From Escherichia coli W3110 Into the Site of α-acetolactate Decarboxylase Gene budA_{Bl}

The acetohydroxyacid isomeroreductase cofactor preference mutant protein coding gene ilvC^{M(L67E,R68F,K75E)} had a sequence length of 1,476 bases, and a nucleotide sequence as shown in SEQ ID NO.9.

The steps for constructing and operating a gene replacement vector for inserting the acetohydroxyacid isomeroreductase cofactor preference mutant protein coding gene ilvC^{M(L67E,R68F,K75E)} derived from Escherichia coli W3110 into the site of α-acetolactate decarboxylase gene budA_{Bl} referred to the operation steps of replacing the gene budA with the gene ilvC^{M(L67E,R68F,K75E)} in the step 1.2.3 of Example 1.2.

The primers for amplifying the recombinant fragment were designed as follows:

budA_{Bl}:ilvC^{M(L67E,R68F,K75E)}1-f:
(SEQ ID NO. 222)
5′-CCGGGGCCCTGGGGTGGCTTTGCCGTGGG-3′
(SmaI)
budA_{Bl}:ilvC^{M(L67E,R68F,K75E)}1-r:
(SEQ ID NO. 223)
5′-GTATTGAAGTAGTTAGCCATTTTCCTCTTTTCACTCCCTT-3′
budA_{Bl}::ilvC^{M(L67E,R68F,K75E)}2-f:
(SEQ ID NO. 224)
5′-AAGGGAGTGAAAAGAGGAAAATGGCTAACTACTTCAATAC-3′
budA_{Bl}::ilvC^{M(L67E,R68F,K75E)}2-r:
(SEQ ID NO. 225)
5′-GCTTTACGCCAGGACGCGCGCTCCTCGGCAATCGCTTC
TTTAAACTCAGCGTAGGAGATATCGAGAC-3′
budA_{Bl}:ilvC^{M(L67E,R68F,K75E)}3-f:
(SEQ ID NO. 226)
5′-GTCTCGATATCTCCTACGCTGAGTTTAAAGAAGCGATT
GCCGAGGAGCGCGCGTCCTGGCGTAAAGC-3′
budA_{Bl}:ilvC^{M(L67E,R68F,K75E)}3-r:
(SEQ ID NO. 227)
5′-TTCAAAGAGGGCTTTTTCATTTAACCCGCAACAGCAATAC-3′
budA_{Bl}:ilvC^{M(L67E,R68F,K75E)}4-f:
(SEQ ID NO. 228)
5′-GTATTGCTGTTGCGGGTTAAATGAAAAAGCCCTCTTTGAA-3′
budA_{Bl}:ilvC^{M(L67E,R68F,K75E)}4-r:
(SEQ ID NO. 229)
5′-TGCGGATCCTTGAAGCGATCAGAAGCTCAGGGAA-3′
(BamHI)

The final resulting recombinant Bacillus licheniformis was named Bacillus licheniformis VBL-3, with the genotype of Bacillus licheniformis 10-1-A Δpox_{Bl} Δpta_{Bl} ΔfrdA_{Bl} ΔpflB_{Bl} ΔbudC_{Bl} ΔgldA_{Bl} ΔldhD_{Bl}::ilvD ΔadhE_{Bl}::bcd ΔbudA_{Bl}::ilvC^{M(L67E,R68F,K75E)}.

5.3 Exogenous Introduction of the Acetohydroxyacid Isomeroreductase Coding Gene ilvC Derived From Escherichia coli W3110, the Branched-Chain Amino Acid Transporter Gene brnFE Derived From Corynebacterium glutamicum ATCC13869, and the α-acetolactate Synthase Gene alsS Derived From Bacillus subtilis 168 to Improve L-valine Synthesis

The branched-chain amino acid transporter gene brnFE had a sequence length of 1,079 bases, and a nucleotide sequence as shown in SEQ ID NO.11. The α-acetolactate synthase gene alsS had a sequence length of 1,713 bases, and a nucleotide sequence as shown in SEQ ID NO.12. The acetohydroxyacid isomeroreductase gene ilvC had a sequence length of 1476 bases, and a nucleotide sequence as shown in SEQ ID NO.14.

5.3.1 Introduction of the Branched-Chain Amino Acid Transporter Gene brnFE Derived From Corynebacterium glutamicum ATCC13869 Into the Site of the Pyruvate Formate Lyase Gene pflB_{Bl} to Enhance L-valine Efflux

The steps for constructing and operating a gene replacement vector for inserting the branched-chain amino acid transporter gene brnFE derived from Corynebacterium glutamicum ATCC13869 into the site of the pyruvate formate lyase gene pflB_{Bl} referred to the operation steps of inserting the gene puDHT into the site of the gene ldhD in the step 1.2.1 of Example 1.

The primers for amplifying the recombinant fragment were designed as follows:

pflB_{Bl}::brnFE1-f:
(SEQ ID NO. 230)
5′-CCGGGGCCCTGACTTCTCCCATTGCAGCA-3′
(SmaI)
pflB_{Bl}::brnFE1-r:
(SEQ ID NO. 231)
5′-ATCTCTTGCGTTTTTTGCACTTAAATCCCCCTCTTTTTCA-3′
pflB_{Bl}::brnFE2-f:
(SEQ ID NO. 232)
5′-TGAAAAAGAGGGGGATTTAAGTGCAAAAAACGCAAGAGAT-3′
pflB_{Bl}::brnFE2-r:
(SEQ ID NO. 233)
5′-CGCGCTCCGCTTATTGCTCGTTAGAAAAGATTCACCAGTC-3′
pflB_{Bl}::brnFE3-f:
(SEQ ID NO. 234)
5′-GACTGGTGAATCTTTTCTAACGAGCAATAAGCGGAGCGCG-3′
pflB_{Bl}::brnFE3-r:
(SEQ ID NO. 235)
5′-TGCGGATCCGGCATTCCTGTCAGGTTGATATGTT-3′
(BamHI)

5.3.2 Introduction of the α-acetolactate Synthase Gene alsS Derived From Bacillus subtilis 168 Into the Site of 2,3-butanediol Dehydrogenase Gene budC_{Bl} to Improve the α-acetolactate Synthesis Efficiency and Inhibit 2,3-butanediol Synthesis

The steps for constructing and operating a gene replacement vector for inserting the α-acetolactate synthase gene alsS derived from Bacillus subtilis 168 into the site of 2,3-butanediol dehydrogenase gene budC_{Bl} referred to the operation steps for inserting the gene puDHT into the site of the gene ldhD in the step 1.2.1 of Example 1.

The primers for amplifying the recombinant fragment were designed as follows:

budC_{Bl}::alsS1-f:
(SEQ ID NO. 236)
5′-CCGGGGCCCAAAGCGCATGTTTTAAAACC-3′
(SmaI)
budC_{Bl}::alsS1-r:
(SEQ ID NO. 237)
5′-TCTTTTGTTGCTTTTGTCAAATAAACATCTTCTTTCTATA-3′
budC_{Bl}::alsS2-f:
(SEQ ID NO. 238)
5′-TATAGAAAGAAGATGTTTATTTGACAAAAGCAACAAAAGA-3′
budC_{Bl}::alsS2-r:
(SEQ ID NO. 239)
5′-TATAGAATATAATTTTAAAACTAGAGAGCTTTCGTTTTCA-3′
budC_{Bl}::alsS3-f:
(SEQ ID NO. 240)
5′-TGAAAACGAAAGCTCTCTAGTTTTAAAATTATATTCTATA-3′
budC_{Bl}::alsS3-r:
(SEQ ID NO. 241)
5′-TGCGGATCCTTGAAGCGATCAGAAGCTCAGGGAA-3′
(BamHI)

5.3.3 Introduction of the Acetohydroxyacid Isomeroreductase Coding Gene ilvC Derived From Escherichia coli W3110 Into the Site of Glycerol Dehydrogenase Gene gldA_{Bl} to Improve L-valine Synthesis

The steps for constructing and operating a gene replacement vector for inserting the acetohydroxyacid isomeroreductase gene ilvC derived from Escherichia coli W3110 into the site of glycerol dehydrogenase gene gldA_{Bl} referred to the operation steps of inserting the gene puDHT into the site of the gene ldhD in the step 1.2.1 of Example 1.2.

The primers for amplifying the recombinant fragment were designed as follows:

gldA_{Bl}::ilvC1-f:
(SEQ ID NO. 242)
5′-ATTTAGATCTAACAAGCCGCGTCATTCAAG-3′
(BglII)
gldA_{Bl}::ilvC1-r:
(SEQ ID NO. 243)
5′-GTATTGAAGTAGTTAGCCATGGTAATTCCCCCTTCACTAT-3′
gldA_{Bl}::ilvC2-f:
(SEQ ID NO. 244)
5′-ATAGTGAAGGGGGAATTACCATGGCTAACTACTTCAATAC-3′
gldA_{Bl}::ilvC2-r:
(SEQ ID NO. 245)
5′-CGGAAACGGCTTTTCGTCTATTAACCCGCAACAGCAATAC-3′
gldA_{Bl}::ilvC3-f:
(SEQ ID NO. 246)
5′-GTATTGCTGTTGCGGGTTAATAGACGAAAAGCCGTTTCCG-3′
gldA_{Bl}::ilvC3-r:
(SEQ ID NO. 247)
5′-TACCGTGGATCCGCTTTAAG-3′
(BamHI)

The final resulting recombinant Bacillus licheniformis was named Bacillus licheniformis VBL-6, with the genotype of Bacillus licheniformis 10-1-A Δpox_{Bl} Δpta_{Bl} ΔfrdA_{Bl} ΔldhD_{Bl}::ilvD ΔadhE_{Bl}::bcd ΔbudA_{Bl}::ilvC^{M(L67E,R68F,K75E)} ΔpflB_{Bl}::brnFE ΔbudC_{Bl}::alsS ΔgldA_{Bl}::ilvC.

5.4 Fed-Batch Fermentation of Engineered Strain of Bacillus licheniformis VBL-6 Using Glucose as a Substrate to Produce L-valine
(1) Plate culture: the recombinant Bacillus licheniformis VBL-6 was streaked onto an LB medium containing agar in a mass-volume ratio of 1.6-1.8% and cultured at 50±1° C. for 10±1 hours;
(2) Seed culture: under sterile conditions, a single colony was picked from the plate in the step (1) with a tip of a sterile pipette, then inoculated into 5 mL of an LB liquid medium, and cultured with oscillating in a shaker at 50±1° C. for 10±1 hours; and then inoculated into 100 mL of an LB liquid medium at an inoculation amount of 1% (v/v) and cultured with oscillating in a shaker at 50±1° C. for 10±1 hours;
(3) 7.5 L fermentation tank culture: under sterile conditions, the bacterial solution obtained in the step (2) was taken and inoculated into a fermentation medium containing 50-60 g/L of glucose at an inoculation amount of 5% (v/v). The fermentation conditions were as follows: a liquid loading volume of 5 L, a culture temperature of 50±1° C., a culture manner of stirring culture, a stirring rotation speed of 500±50 revolutions per minute, a ventilation volume of 1.0±0.1 vvm, and a pH maintained at 7.0±0.1 by adjusting with ammonia water, and among the fermentations, samples were taken every 4 hours to detect OD₆₂₀ₙₘ and the concentration of the glucose in the fermentation sample, and glucose powder was added according to the glucose concentration to maintain the glucose concentration at 40-50 g/L; and at the same time the fermentation sample was subjected to high performance liquid chromatography analysis to determine the concentration of L-valine in the fermentation broth. When glucose was not consumed any more, fermentation was stopped and L-valine was obtained from the fermentation broth.

The results showed that the recombinant strain B. licheniformis VBL-6 was cultured for 32 h, which consumed 183.0 g/L of glucose, and the concentration of L-valine reached 45.1 g/L, the production intensity reached 1.41 g/L/h, and the yield of L-valine reached 0.246 g/g.

The methods for detecting the substrate glucose and the product L-valine as well as the formula of the LB medium described in the aforementioned steps were consistent with those in Example 2.

The formula of the fermentation medium described in the aforementioned step (3) was: 12 g/L of yeast powder, 6.5 g/L of anhydrous sodium acetate (C₂H₃NaO₂), 1 g/L of ammonium citrate (C₆H₁₇N₃O₇), 2 g/L of K₂HPO₄, 0.25 g/L of MgSO₄·7H₂O, 10 mL of a 100× trace element solution; wherein the formula of the 100× trace element solution is: 2.25 g/L of FeSO₄, 0.75 g/L of ZnSO₄, and 0.38 g/L of MnSO₄.

The above description of the embodiments is to facilitate those of ordinary skills in the art to understand and use the present invention. It is obvious that those skilled in the art can easily make various modifications to these embodiments and apply the general principles described herein to other embodiments without inventive efforts. Therefore the present invention is not limited to the aforementioned embodiments. The improvements and modifications made by those skilled in the art according to the principle of the present invention without departing from the scope of the present invention should be within the claimed scope of the present invention.

1. A method for constructing an L-valine producing strain, comprising genetically engineering a 2,3-butanediol or acetoin producing strain as a starting strain to increase L-valine production thereof.

2. The method according to claim 1, wherein the strain is subjected to the following engineering: 1) increasing the synthesis of α-acetolactate; and 2) introducing an exogenous L-valine biosynthetic pathway.

3. The method according to claim 1, wherein the starting strain is selected from microorganisms of the genera Klebsiella, Enterobacter, Bacillus, Corynebacterium and Vibrio.

4. The method according to claim 3, wherein the starting strain is selected from Klebsiella oxytoca, Enterobacter cloacae, Escherichia coli, Vibrio natriegens, Corynebacterium glutamicum and Bacillus licheniformis.

5. The method according to claim 2, wherein the increasing the synthesis of α-acetolactate comprises: i) inhibiting the synthesis of acetoin and/or 2,3-butanediol; and/or ii) inhibiting the synthesis of acetic acid, formic acid, ethanol, succinic acid and/or lactic acid.

6. The method according to claim 5, wherein the inhibiting the synthesis of acetoin and/or 2,3-butanediol comprises knocking out or knocking down one or more of the following coding genes in the starting strain: an α-acetolactate decarboxylase coding gene budA, a 2,3-butanediol dehydrogenase coding gene budC and a glycerol dehydrogenase coding gene gldA.

7. The method according to claim 5, wherein the inhibiting the synthesis of acetic acid, formic acid, ethanol, succinic acid and/or lactic acid comprises knocking out or knocking down one or more of the following coding genes in the starting strain: a pyruvate oxidase coding gene pox, a phosphotransacetylase coding gene pta, a fumarate reductase subunit A coding gene frdA, a lactate dehydrogenase coding gene ldh, a pyruvate formate lyase coding gene pflB and an ethanol dehydrogenase coding gene adhE.

8. The method according to claim 2, wherein the introducing an exogenous L-valine biosynthetic pathway comprises introducing coding sequences of one or more of the following genes into the starting strain: a dihydroxyacid dehydratase gene, an L-leucine dehydrogenase gene and an acetohydroxyacid isomeroreductase gene.

9. The method according to claim 8, wherein the introducing an exogenous L-valine biosynthetic pathway comprises introducing coding sequence of one or more of the following genes into the starting strain: a dihydroxyacid dehydratase gene puDHT, a dihydroxyacid dehydratase gene dhaD, a dihydroxyacid dehydratase gene ilvD, an L-leucine dehydrogenase gene bcd, and an acetohydroxyacid isomeroreductase cofactor preference mutant protein gene ilvC^{M}.

10. The method according to claim 2, wherein the engineering further comprises optimizing L-valine synthetic flux and/or enhancing L-valine efflux in the starting strain.

11. The method according to claim 10, wherein the optimizing L-valine synthetic flux and/or enhancing L-valine efflux in the starting strain comprises introducing coding sequences of one or more of the following genes into the starting strain: the acetohydroxyacid isomeroreductase cofactor preference mutant protein gene ilvC^{M}, a branched-chain amino acid transporter gene brnFE, a branched-chain amino acid transporter gene ygaZH, an α-acetolactate synthase gene alsS, an α-acetolactate synthase gene budB, an acetohydroxyacid isomeroreductase gene ilvC, a dihydroxyacid dehydratase gene dhaD, and a dihydroxyacid dehydratase gene ilvD.

12. The method according to claim 8, and wherein the introducing coding sequences into the starting strain comprises integrating the coding sequences into a genome of the starting strain or expressing the coding sequence in the starting strain in a plasmid form; preferably, the introducing comprises introducing a single copy or multiple copies of the coding sequences of the genes; and preferably, the coding sequences of the genes are introduced in the form of respective single gene expression fragments or in the form of tandem expression fragments of the coding sequences of the genes.

13. The method according to claim 9, wherein the dihydroxyacid dehydratase gene puDHT is derived from Paralcaligenes ureilyticus, the dihydroxyacid dehydratase gene dhaD is derived from Sulfolobus solfataricus, the dihydroxyacid dehydratase gene ilvD is derived from Escherichia coli, the L-leucine dehydrogenase gene bcd is derived from Bacillus subtilis, and the acetohydroxyacid isomeroreductase cofactor preference mutant protein gene ilvC^{M} is derived from Escherichia coli.

14. The method according to claim 11, wherein the acetohydroxyacid isomeroreductase cofactor preference mutant protein gene ilvC^{M} is derived from Escherichia coli, the branched-chain amino acid transporter gene brnFE is derived from Corynebacterium glutamicum, the branched-chain amino acid transporter gene ygaZH is derived from Escherichia coli, the acetohydroxyacid isomeroreductase gene ilvC is derived from Escherichia coli, the dihydroxyacid dehydratase gene dhaD is derived from Sulfolobus solfataricus, the dihydroxyacid dehydratase gene ilvD is derived from Escherichia coli, the α-acetolactate synthase gene alsS is derived from Bacillus subtilis, and the α-acetolactate synthase gene budB is derived from Klebsiella pneumoniae.

15. An L-valine producing strain, which is constructed by the method according to claim 1.

16. The strain according to claim 15, wherein the strain is Klebsiella oxytoca with a deposit number of CCTCC M 20221743.

17. (canceled)

18. (canceled)

19. A method for producing L-valine, comprising the following steps:
1) providing the L-valine producing strain according to claim 15;
2) culturing the strain at 30-50° C. for 10-11 hours to provide a seed; and
3) fermenting the seed with glucose as a substrate at 30-50° C., a pH value of 6.0-7.0 and a ventilation volume of 0.5-1.6 vvm to obtain L-valine.

20. The method according to claim 19, wherein in the step 3), the inoculation amount of the seed is an OD₆₂₀ₙₘ value reaching 0.2-0.8; preferably, the concentration of glucose is 40-60 g/L; and preferably, the fermentation culture is a stirred culture with a stirring rate of 300-550 rpm.
